SEQUENCE LISTING (39 sequences)

SEQ ID NO: 1 - protein, 450 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAKEAPYYAKDYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, Homo sapiens
EIVLTQSPGTLSLSPGERATLSCRASQSVSSDYLAWYQQKPGQAPRLLIYGASSRATGIPDRFSGSGSGTDFTLTISRLEPEDFAVYYCQQYVSYWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 450 aa, Homo sapiens
QVQLVESGGGVVQPGRSLRLSCAASGFTFSSYGMHWVRQAPGKGLEWVAGIWWDGSNEKYADSVKGRFTISRDNSKNTLYLQMNSLRAEDTAVYYCAKEAPYYAKDYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 214 aa, Homo sapiens
EIVLTQSPGTLSLSPGERATLSCRASQSVSSDYLAWYQQKPGQAPRLLIYGASRRATGIPDRFSGSGSGTDFTLTISRLEPEDFAVYYCQQYVSYWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 446 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGGHGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 215 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYHHSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 446 aa, Homo sapiens
EVQLVESGGGLVQPGGSLRLSCAASGFTFSGRSMNWVRQAPGKGLEWVSYISSDSRTIYYADSVKGRFTISRDNAKNSLYLQMNSLRAEDTAVYYCARGGHGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 215 aa, Homo sapiens; identical to SEQ ID NO: 6

SEQ ID NO: 9 - protein, 121 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAKEAPYYAKDYMDVWGKGTTVTVSS

SEQ ID NO: 10 - protein, 107 aa, Homo sapiens
EIVLTQSPGTLSLSPGERATLSCRASQSVSSDYLAWYQQKPGQAPRLLIYGASSRATGIPDRFSGSGSGTDFTLTISRLEPEDFAVYYCQQYVSYWTFGGGTKVEIK

SEQ ID NO: 11 - protein, 121 aa, Homo sapiens
QVQLVESGGGVVQPGRSLRLSCAASGFTFSSYGMHWVRQAPGKGLEWVAGIWWDGSNEKYADSVKGRFTISRDNSKNTLYLQMNSLRAEDTAVYYCAKEAPYYAKDYMDVWGKGTTVTVSS

SEQ ID NO: 12 - protein, 107 aa, Homo sapiens
EIVLTQSPGTLSLSPGERATLSCRASQSVSSDYLAWYQQKPGQAPRLLIYGASRRATGIPDRFSGSGSGTDFTLTISRLEPEDFAVYYCQQYVSYWTFGGGTKVEIK

SEQ ID NO: 13 - protein, 117 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGGHGYFDLWGRGTLVTVSS

SEQ ID NO: 14 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYHHSPLTFGGGTKVEIK

SEQ ID NO: 15 - protein, 117 aa, Homo sapiens
EVQLVESGGGLVQPGGSLRLSCAASGFTFSGRSMNWVRQAPGKGLEWVSYISSDSRTIYYADSVKGRFTISRDNAKNSLYLQMNSLRAEDTAVYYCARGGHGYFDLWGRGTLVTVSS

SEQ ID NO: 16 - protein, 108 aa, Homo sapiens; identical to SEQ ID NO: 14

SEQ ID NO: 17 - protein, 9 aa, Homo sapiens
FTFSSYGMH

SEQ ID NO: 18 - protein, 17 aa, Homo sapiens
VIWYDGSNKYYADSVKG

SEQ ID NO: 19 - protein, 12 aa, Homo sapiens
EAPYYAKDYMDV

SEQ ID NO: 20 - protein, 12 aa, Homo sapiens
RASQSVSSDYLA

SEQ ID NO: 21 - protein, 7 aa, Homo sapiens
GASSRAT

SEQ ID NO: 22 - protein, 8 aa, Homo sapiens
QQYVSYWT

SEQ ID NO: 23 - protein, 17 aa, Homo sapiens
GIWWDGSNEKYADSVKG

SEQ ID NO: 24 - protein, 7 aa, Homo sapiens
GASRRAT

SEQ ID NO: 25 - protein, 9 aa, Homo sapiens
FTFSSYSMN

SEQ ID NO: 26 - protein, 17 aa, Homo sapiens
YISSSSSTIYYADSVKG

SEQ ID NO: 27 - protein, 8 aa, Homo sapiens
GGHGYFDL

SEQ ID NO: 28 - protein, 12 aa, Homo sapiens
RASQSVSSSYLA

SEQ ID NO: 29 - protein, 9 aa, Homo sapiens
QQYHHSPLT

SEQ ID NO: 30 - protein, 9 aa, Homo sapiens
FTFSGRSMN

SEQ ID NO: 31 - protein, 17 aa, Homo sapiens
YISSDSRTIYYADSVKG

SEQ ID NO: 32 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 107 aa, Homo sapiens [sequence omitted]

SEQ ID NO: 34 - DNA, 363 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggcggtgtactactgcgccaaggaagctccctactacgctaaagattacatggacgtatggggcaagggtacaactgtcaccgtctcctca

SEQ ID NO: 35 - DNA, 321 bp, Homo sapiens
gaaattgtgttgacgcagtctccaggcaccctgtctttgtctccaggggaaagagccaccctctcctgcagggccagtcagagtgttagcagcgactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtacgtcagttactggacttttggcggagggaccaaggttgagatcaaa

SEQ ID NO: 36 - DNA, 351 bp, Homo sapiens
gaggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagctgaggacacggcggtgtactactgcgccagaggtggacacggatatttcgacctatgggggagaggtaccttggtcaccgtctcctca

SEQ ID NO: 37 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtaccaccacagtcctctcacttttggcggagggaccaaggttgagatcaaa

SEQ ID NO: 38 - protein, 1255 aa, Homo sapiens [sequence omitted]

SEQ ID NO: 39 - protein, 630 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

(19) United States
(12) Patent Application Publication
(10) Pub. No.: US 2018/0271996 A1
(43) Pub. Date: Sep. 27, 2018

(54) COMBINATION THERAPIES OF HER2-TARGETED ANTIBODY-DRUG CONJUGATES

(71) Applicants: Mersana Therapeutics, Inc., Cambridge, MA (US); Millennium Pharmaceuticals Inc., Cambridge, MA (US)

(72) Inventors: Natalya D. BODYAK, Boston, MA (US); Donald A. BERGSTROM, Winchester, MA (US); Marc HYER, Swampscott, MA (US); Timothy B. LOWINGER, Carlisle, MA (US); Marina PROTOPOPOVA, Newton, MA (US); Ole Petter VEIBY, Westborough, MA (US); Aleksandr V. YURKOVETSKIY, Littleton, MA (US); Qing Xiu ZHANG, Lexington, MA (US)

(73) Assignees: Mersana Therapeutics, Inc., Cambridge, MA (US); Millennium Pharmaceuticals Inc., Cambridge, MA (US)

(21) Appl. No.: 15/906,297

(22) Filed: Feb. 27, 2018

Related U.S. Application Data
(60) Provisional application No. 62/479,914, filed on Mar. 31, 2017.
(60) Provisional application No. 62/465,028, filed on Feb. 28, 2017.

(51) Int. Cl. A61K 47/68; C07K 16/32; A61K 39/395; A61K 47/60; A61P 35/00

(52) U.S. Cl. CPC A61K 47/6883; A61K 2039/507; A61K 39/39558; A61K 39/3955; A61K 47/6851; A61K 47/6855; A61K 47/6863; A61K 47/6857; A61K 47/6869; A61K 47/6809; A61K 47/6817; A61K 47/6803; A61K 47/60; A61P 35/00; C07K 2317/32; C07K 2317/565; C07K 2317/56; C07K 16/32

(57) ABSTRACT

Disclose herein are combinations comprising HER2-targeted antibody-drug conjugates and immune checkpoint inhibitors and methods of using such combinations in a variety of therapeutic, diagnostic, and prophylactic indications.

### RELATED APPLICATIONS

This application claims priority to, and the benefit of, U.S. provisional application Nos. 62/465,028, filed Feb. 28, 2017 and 62/479,914, filed Mar. 31, 2017, under 35 USC § 119(e). The contents of each of these applications are hereby incorporated by reference in their entireties.

### REFERENCE TO SEQUENCE LISTING

The present application is being filed along with a Sequence Listing in electronic format. The Sequence Listing is provided as a file entitled MRSN-012_001US Seq Listing_ST25.TXT, created Jun. 1, 2018, which is 56 Kb in size. The information in the electronic format of the Sequence Listing is incorporated herein by reference in its entirety.

### FIELD

This disclosure relates generally to the combinations comprising HER2-targeted antibody-drug conjugates and immune checkpoint inhibitors, and to methods of using these combinations as therapeutics and/or diagnostics.

### BACKGROUND

Members of the ErbB family of receptor tyrosine kinases are important mediators of cell growth, differentiation and survival. The receptor family includes four distinct members, including epidermal growth factor receptor (EGFR or ErbB1), HER2 (ErbB2 or p185^{neu}), HER3 (ErbB3) and HER4 (ErbB4 or tyro2). Both homo- and heterodimers are formed by the four members of the EGFR family, with HER2 being the preferred and most potent dimerization partner for other ErbB receptors (Graus-Porta et al., Embo 3 1997; 16:1647-1655; Tao et al., J Cell Sci 2008; 121:3207-3217). HER2 has no known ligand, but can be activated via homodimerization when overexpressed, or by heterodimerization with other, ligand occupied ErbB receptors.

The HER2 gene (also known as HER2/neu and ErbB2 gene) is amplified in 20-30% of early-stage breast cancers, which makes it overexpress epidermal growth factor (EGF) receptors in the cell membrane (Bange, et al., Nature Medicine 7 (5): 548-552). Besides breast cancer, HER2 expression has also been associated with other human carcinoma types, including non-small cell lung cancer, ovarian cancer, gastric cancer, prostate cancer, bladder cancer, colon cancer, esophageal cancer and squamous cell carcinoma of the head & neck (Garcia de Palazzo et al., Int J Biol Markers 1993; 8:233-239; Ross et al., Oncologist 2003; 8:307-325; Osman et al., J Urol 2005; 174:2174-2177; Kapitanovic et al., Gastroenterology 1997; 112:1103-1113; Turken et al., Neoplasma 2003; 50:257-261; and Oshima et al., Int J Biol Markers 2001; 16:250-254).

Trastuzumab (Herceptin®) is a recombinant, humanized monoclonal antibody directed against domain IV of the HER2 protein, thereby blocking ligand-independent HER2 homodimerization, and to a lesser extend heterodimerization of HER2 with other family members in cells with high HER2 overexpression (Cho et al., Nature 2003; 421:756-760 and Wehrman et al., Proc Natl Acad Sci USA 2006; 103:19063-19068). Herceptin® has been approved both for first-line and adjuvant treatment of HER2 overexpressing metastatic breast cancer, either in combination with chemotherapy, or as a single agent following one or more chemotherapy regimens. Trastuzumab has been found to be effective only in 20-50% of HER2 overexpressing breast tumor patients and many of the initial responders show relapse after a few months (Dinh et al., Clin Adv Hematol Oncol 2007; 5:707-717).

Pertuzumab (Omnitar/Perjeta® also called 2C4) is another humanized monoclonal antibody directed against domain II of the HER2 protein, resulting in inhibition of ligand-induced heterodimerization (i.e., HER2 dimerizing with another member of the ErbB family to which a ligand has bound); a mechanism reported to not strictly require high HER2 expression levels (Franklin et al., Cancer Cell 2004; 5:317-328.). Pertuzumab is approved for the treatment of HER2-positive metastatic breast cancer, in combination with trastuzumab and docetaxel.

A HER2 antibody drug conjugate (ADC), Trastuzumab emtansine (ado-trastuzumab emtansine, Kadcyla®) is an antibody-drug conjugate consisting of the monoclonal antibody trastuzumab (Herceptin) linked to the cytotoxic agent mertansine (DM1). Kadcyla® (ado-trastuzumab emtansine) as a single agent, has been approved for the treatment of patients with HER2-positive (HER2+), metastatic breast cancer (MBC) who previously received trastuzumab and a taxane, separately or in combination.

Combination therapy in which two or more drugs are used in certain dosing regimen or administration form, can enhance potency by exploiting additive or synergistic effects in the biological activity of the two or more drugs.

The complex mechanisms regulating the function of HER2 warrant further research on new and optimized therapeutic strategies against this proto-oncogene, including new combination therapies.

### SUMMARY OF THE DISCLOSURE

The present disclosure provides, inter alia, a combination comprising a HER2-targeted antibody-drug conjugate and an immunomodulatory therapy, e.g., an immuno-oncology agent such as an immune checkpoint inhibitor, wherein the conjugate comprises an antibody or antigen binding fragment thereof that specifically binds to an epitope of the human HER2 receptor and one or more therapeutic or diagnostic agents (D), wherein each D is independently connected directly or indirectly to the antibody or antigen binding fragment thereof. For example, in some embodiments, the HER2-targeted antibody-drug conjugate enhances the efficacy of the immune checkpoint inhibitor.

In some embodiments, the HER2 antibody conjugate described herein includes a HER2 antibody or antigen binding fragment thereof connected directly or indirectly to one or more therapeutic or diagnostic agents (D). In some embodiments, the HER2 antibody conjugate also includes one or more polymeric scaffolds connected to the antibody or antigen binding fragment thereof, wherein each of the one or more D is independently connected to the antibody or antigen binding fragment thereof via the one or more polymeric scaffolds. In certain embodiments, the HER2 antibody or antigen binding fragment thereof used for the conjugates described herein is isolated antibody or antigen binding fragment thereof.

In some embodiments, each of the one or more polymeric scaffolds that are connected to the HER2 antibody or antigen binding fragment thereof, independently, comprises poly(1-hydroxymethylethylene hydroxymethyl-formal) (PHF) having a molecular weight ranging from about 2 kDa to about 40 kDa.

In some embodiments, each of the one or more polymeric scaffolds independently is of Formula (Ic):

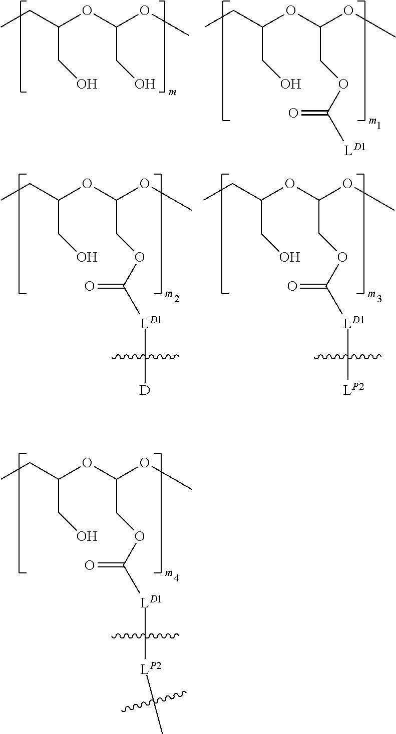

wherein:

L^{D1} is a carbonyl-containing moiety;

each occurrence of

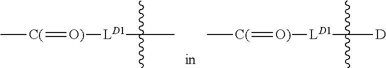

is independently a first linker that contains a biodegradable bond so that when the bond is broken, D is released in an active form for its intended therapeutic effect; and the

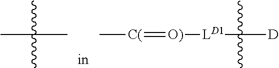

between L^{D1} and D denotes direct or indirect attachment of D to L^{D1};

each occurrence of

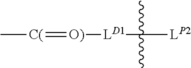

is independently a second linker not yet connected to the antibody or antigen binding fragment thereof, in which L^{P2} is a moiety containing a functional group that is yet to form a covalent bond with a functional group of the antibody or

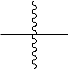

antigen binding fragment thereof, and the between L^{D1} and L^{P2} denotes direct or indirect attachment of L^{P2} to L^{D1}, and each occurrence of the second linker is distinct from each occurrence of the first linker;

each occurrence of

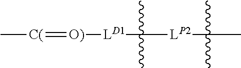

is independently a third linker that connects each D-carrying polymeric scaffold to the antibody or antigen binding fragment thereof, in which the terminal

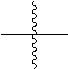

attached to L^{P2} denotes direct or indirect attachment of L^{P2} to the antibody or antigen binding fragment thereof upon formation of a covalent bond between a functional group of L^{P2} and a functional group of the antibody or antigen binding fragment thereof; and each occurrence of the third linker is distinct from each occurrence of the first linker;

m is an integer from 1 to about 300,

m₁ is an integer from 1 to about 140,

m₂ is an integer from 1 to about 40,

m₃ is an integer from 0 to about 18,

m₄ is an integer from 1 to about 10;

the sum of m, m₁, m₂, m₃, and m₄ ranges from about 15 to about 300; and

the total number of L^{P2} connected to the antibody or antigen binding fragment thereof is 10 or less.

The conjugate described herein can include one or more of the following features:

For example, in Formula (Ic), the HER2 antibody or antigen-binding fragment thereof has a molecular weight of 40 kDa or greater (e.g., 60 kDa or greater, 80 kDa or greater, 100 kDa or greater, 120 kDa or greater, 140 kDa or greater, 160 kDa or greater, 180 kDa or greater, or 200 kDa or greater, or about 40-200 kDa, 40-180 kDa, 40-140 kDa, 60-200 kDa, 60-180 kDa, 60-140 kDa, 80-200 kDa, 80-180 kDa, 80-140 kDa, 100-200 kDa, 100-180 kDa, 100-140 kDa, or 140-150 kDa). In some embodiments, the HER2 antibody or antigen-binding fragment thereof includes, by way of non-limiting example, the XMT 1517 antibody, the XMT 1518 antibody, the XMT 1519 antibody and the XMT 1520 antibody described herein.

For example, in Formula (Ic), m₁ is an integer from 1 to about 120 (e.g., about 1-90) and/or m₃ is an integer from 1 to about 10 (e.g., about 1-8).

For example, when the PHF in Formula (Ic) has a molecular weight ranging from about 6 kDa to about 20 kDa (i.e., the sum of m, m₁, m₂, m₃, and m₄ ranging from about 45 to about 150), m₂ is an integer from 2 to about 20, m₃ is an integer from 0 to about 9, m₄ is an integer from 1 to about 10, and/or m₁ is an integer from 1 to about 75 (e.g., m₁ being about 4-45).

For example, when the PHF in Formula (Ic) has a molecular weight ranging from about 8 kDa to about 15 kDa (i.e., the sum of m, m₁, m₂, m₃, and m₄ ranging from about 60 to about 110), m₂ is an integer from 2 to about 15, m₃ is an integer from 0 to about 7, m₄ is an integer from 1 to about 10, and/or m₁ is an integer from 1 to about 55 (e.g., m₁ being about 4-30).

For example, when the PHF in Formula (Ic) has a molecular weight ranging from about 2 kDa to about 20 kDa (i.e., the sum of m, m₁, m₂, m₃, and m₄ ranging from about 15 to about 150), m₂ is an integer from 1 to about 20, m₃ is an integer from 0 to about 10 (e.g., m₃ ranging from 0 to about 9), m₄ is an integer from 1 to about 8, and/or m₁ is an integer from 1 to about 70, and the total number of L^{P2} connected to the antibody or antigen binding fragment thereof ranges from about 2 to about 8 (e.g., about 2, 3, 4, 5, 6, 7, or 8).

For example, when the PHF in Formula (Ic) has a molecular weight ranging from about 3 kDa to about 15 kDa (i.e., the sum of m, m₁, m₂, m₃, and m₄ ranging from about 20 to about 110), m₂ is an integer from 2 to about 15, m₃ is an integer from 0 to about 8 (e.g., m₃ ranging from 0 to about 7), m₄ is an integer from 1 to about 8, and/or m₁ is an integer from 2 to about 50, and the total number of L^{P2} connected to the antibody or antigen binding fragment thereof ranges from about 2 to about 8 (e.g., about 2, 3, 4, 5, 6, 7, or 8).

For example, when the PHF in Formula (Ic) has a molecular weight ranging from about 5 kDa to about 10 kDa, (i.e. the sum of m, m₁, m₂, m₃ and m₄ ranges from about 40 to about 75), m₂ is an integer from about 2 to about 10 (e.g., m₂ being about 3-10), m₃ is an integer from 0 to about 5 (e.g., m₃ ranging from 0 to about 4), m₄ is an integer from 1 to about 8 (e.g., m₄ ranging from 1 to about 5), and/or m₁ is an integer from about 2 to about 35 (e.g., m₁ being about 5-35), and the total number of L^{P2} connected to the antibody or antigen binding fragment thereof ranges from about 2 to about 8 (e.g., about 2, 3, 4, 5, 6, 7, or 8).

For example, each occurrence of D independently is a therapeutic agent having a molecular weight of ≤5 kDa.

For example, each occurrence of D independently is an anti-cancer drug, for example, selected from vinca alkaloids, auristatins, tubulysins, duocarmycins, non-natural camptothecin compounds, maytansinoids, calicheamicin compounds, topoisomerase inhibitors, DNA binding drugs, kinase inhibitors, MEK inhibitors, KSP inhibitors, and analogs thereof. For example, each occurrence of D independently is auristatin E (also known as a derivative of dolastatin-10), auristatin EB (AEB), auristatin EFP (AEFP), monomethyl auristatin E (MMAE), monomethyl auristatin F (MMAF), auristatin F, auristatin F phenylenediamine (AFP), auristatin F hydroxypropyl amide (AF HPA), monomethyl auristatin F hydroxypropyl amide (MMAF HPA), and dolastatin.

For example, each

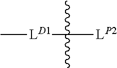

when not connected to the antibody or antigen-binding fragment thereof, independently comprises a terminal group W^{P}, in which each W^{P} independently is:

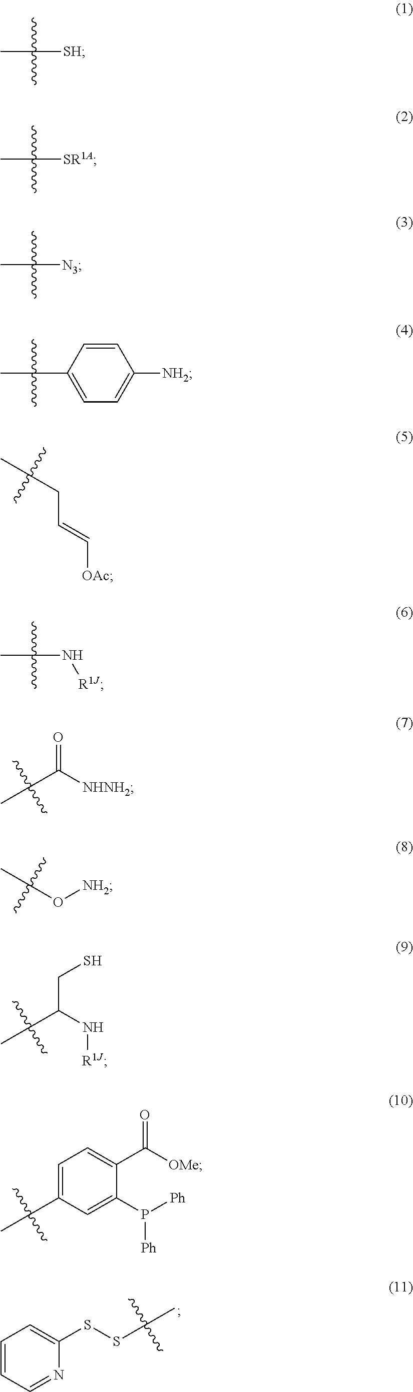

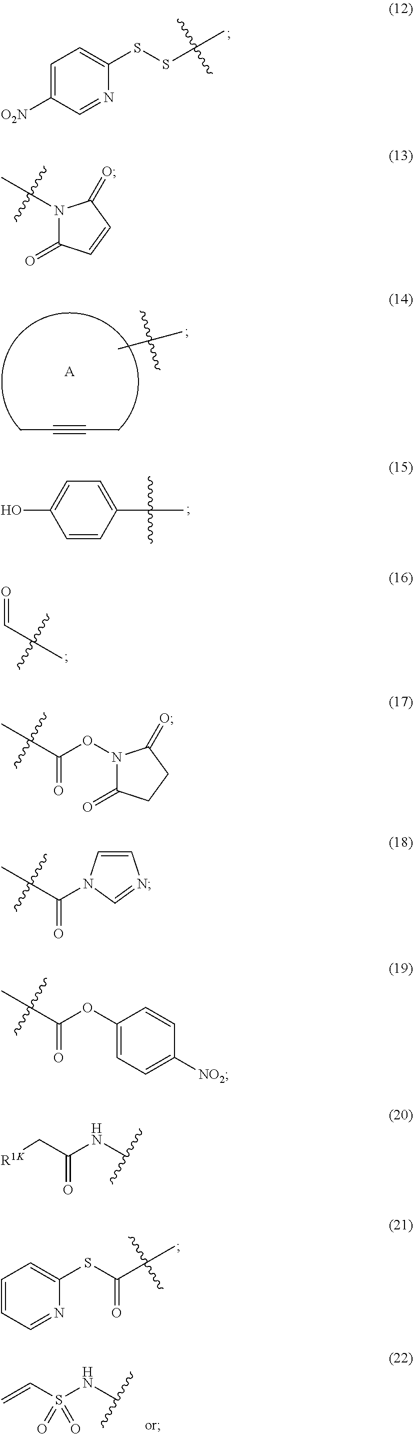

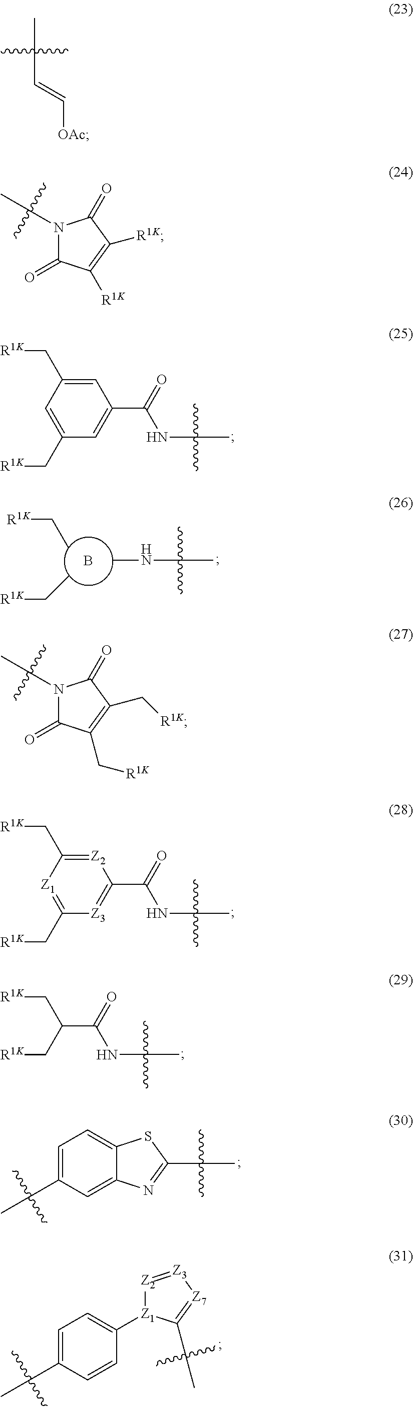

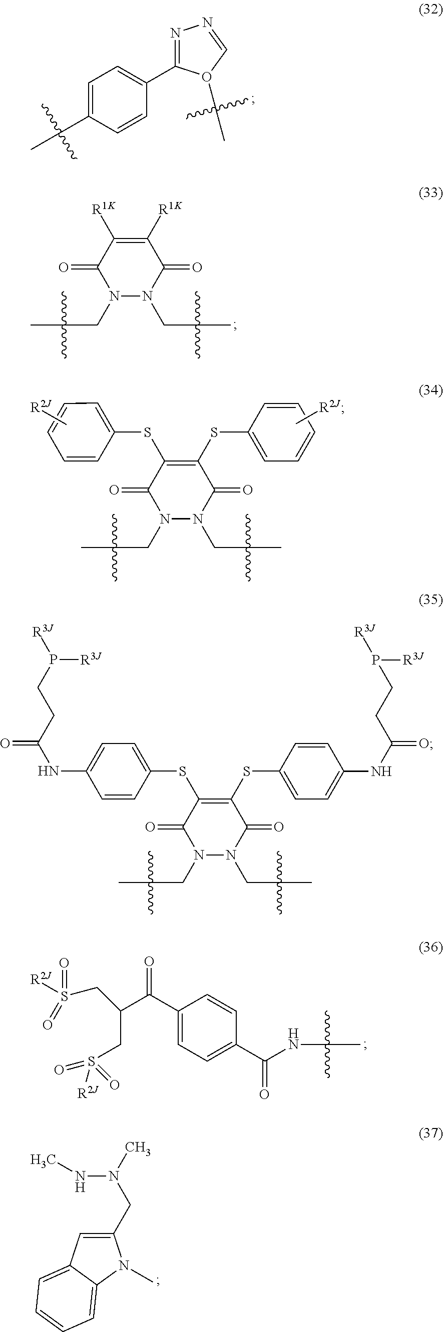

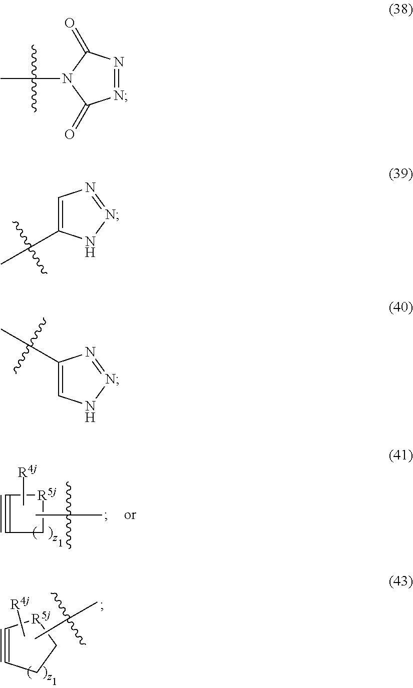

wherein

R^{1K} is a leaving group;

R^{1A} is a sulfur protecting group;

ring A is cycloalkyl or heterocycloalkyl;

ring B is cycloalkyl, heterocycloalkyl, aryl, or heteroaryl;

R^{1J} is hydrogen, an aliphatic, heteroaliphatic, carbocyclic, or heterocycloalkyl moiety;

R^{2J} is hydrogen, an aliphatic, aryl, heteroaliphatic, or carbocyclic moiety;

R^{3J} is C₁₋₆ alkyl;

Z₁, Z₂, Z₃ and Z₇ are each independently a carbon or nitrogen atom;

R^{4j} is hydrogen, halogen, OR, —NO₂, —CN, —S(O)₂R, C₁₋₂₄ alkyl (e.g., C₁₋₆ alkyl), or 6-24 membered aryl or heteroaryl, wherein the C₁₋₂₄ alkyl (e.g., C₁₋₆ alkyl), or 6-24 membered aryl or heteroaryl, is optionally substituted with one or more aryl or heteroaryl; or two R⁴ together form an annelated cycloalkyl, heterocycloalkyl, aryl, or heteroaryl; R is hydrogen, alkyl, heteroalkyl, cycloalkyl, or heterocycloalkyl

R is hydrogen, aliphatic, heteroaliphatic, carbocyclic, or heterocycloalkyl moiety;

R^{5j} is C(R⁴)₂, O, S or NR; and

z₁ is an integer 1, 2, 3, 4, 5, 6, 7, 8, 9 or 10.

For example, each R^{1A} independently is

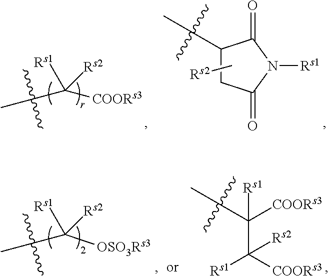

in which r is 1 or 2 and each of R^{s1}, R^{s2}, and R^{s3} is hydrogen, an aliphatic, heteroaliphatic, carbocyclic, or heterocycloalkyl moiety.

For example, the functional group of L^{P2} that is yet to form a covalent bond with a functional group of the antibody or antigen binding fragment thereof is selected from —SR^{p}, —S—S-LG,

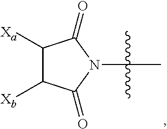

and halo, in which LG is a leaving group, R^{p} is H or a sulfur protecting group, and one of Xₐ and X_{b} is H and the other is a water-soluble maleimido blocking moiety, or Xₐ and X_{b}, together with the carbon atoms to which they are attached for a carbon-carbon double bond. For example, the functional group of L^{P2} that is yet to form a covalent bond is a functional group that is not reacted with a functional group of the antibody or antigen binding fragment thereof, e.g.,

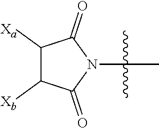

as the functional group of L^{P2}, in which one of Xₐ and X_{b} is H and the other is a water-soluble maleimido blocking moiety, or Xₐ and X_{b}.

For example, L^{D1} comprises —X—(CH₂)ᵥ—C(═O)— with X directly connected to the carbonyl group of

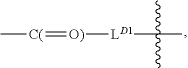

in which X is CH₂, O, or NH, and v is an integer from 1 to 6.

For example, each occurrence of

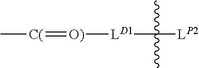

is independently —C(═O)—X—(CH₂)ᵥ—C(═O)—NH—(CH₂)ᵤ—NH—C(═O)—(CH₂)_{w}—(OCH₂)ₓ—NHC(═O)—(CH₂)_{y}-M, in which X is CH₂, O, or NH, each of v, u, w, x and y independently is an integer from 1 to 6, and M is

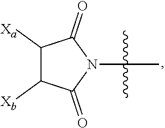

wherein one of Xₐ and X_{b} is H and the other is a water-soluble maleimido blocking moiety, or Xₐ and X_{b}, together with the carbon atoms to which they are attached for a carbon-carbon double bond.

For example, each of v, u, w, x and y is 2.

For example, the ratio between D and the HER2 antibody or antigen-binding fragment thereof ranges from about 25:1 to about 1:1 (e.g., about 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1 or 1:1).

For example, the ratio between D and the HER2 antibody or antigen-binding fragment thereof ranges from about 20:1 to about 1:1 (e.g., about 20:1, 15:1, 10:1, 5:1, 2:1 or 1:1).

For example, the ratio between D and the HER2 antibody or antigen-binding fragment thereof ranges from about 16:1 to about 9:1 (e.g., about 16:1, 15:1, 14:1, 13:1, 12:1, 11:1, 10:1 or 9:1).

For example, the ratio between D and the HER2 antibody or antigen-binding fragment thereof ranges from about 15:1 to about 12:1 (e.g., about 15:1, 14:1, 13:1 or 12:1).

For example, the ratio between D and the HER2 antibody or antigen-binding fragment thereof ranges from about 15:1 to about 10:1 (e.g., about 15:1, 14:1, 13:1, 12:1, 11:1 or 10:1).

For example, the ratio between D and the HER2 antibody or antigen-binding fragment thereof ranges from about 15:1 to about 9:1 (e.g., about 15:1, 14:1, 13:1, 12:1, 11:1, 10:1 or 9:1).

For example, the ratio between D and the HER2 antibody or antigen-binding fragment thereof ranges from about 12:1 to about 9:1 (e.g., about 12:1, 11:1, 10:1 or 9:1).

For example, the ratio between D and the HER2 antibody or antigen-binding fragment thereof ranges from about 12:1 to about 10:1 (e.g., about 12:1, 11:1 or 10:1).

For example, the ratio between the D and the HER2 antibody or antigen-binding fragment thereof ranges from about 6:1 to about 1:1 (e.g., about 6:1, 5:1, 4:1, 3:1, 2:1 or 1:1).

For example, each of the one or more D-carrying polymeric scaffolds independently is of Formula (Id):

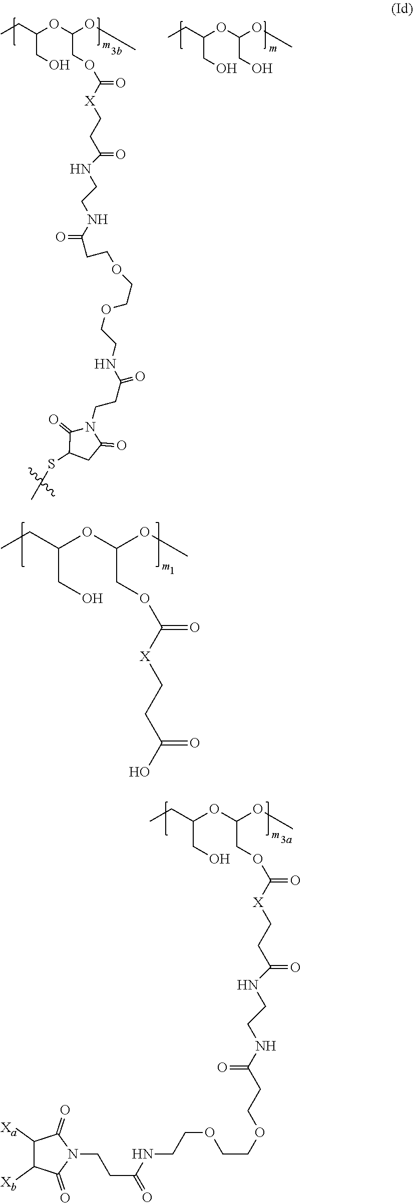

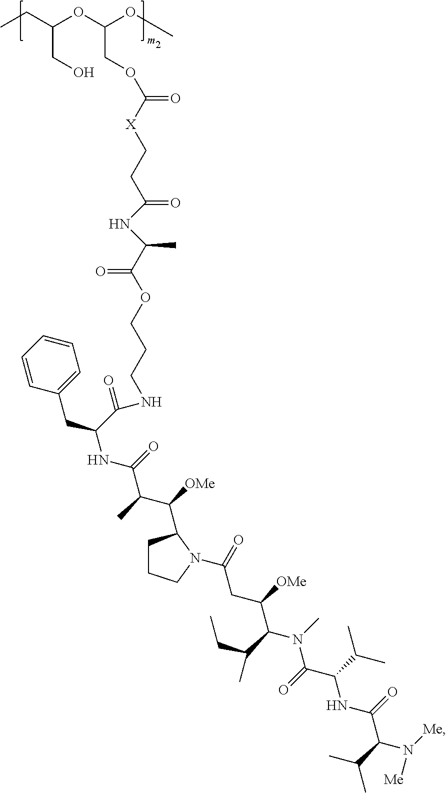

wherein:

m₃ₐ is an integer from 0 to about 17,

m_{3b} is an integer from 1 to about 8, and

the terminal

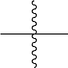

denotes the direct attachment of the one or more polymeric scaffolds to the HER2 antibody or antigen-binding fragment thereof having a molecular weight of 40 kDa or greater.

For example, each of the one or more D-carrying polymeric scaffolds independently is of Formula (Id-1):

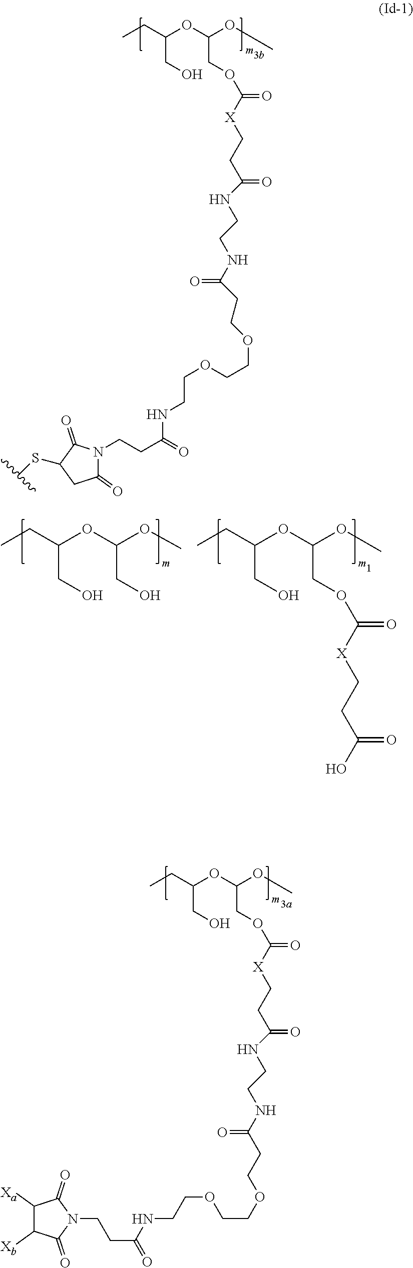

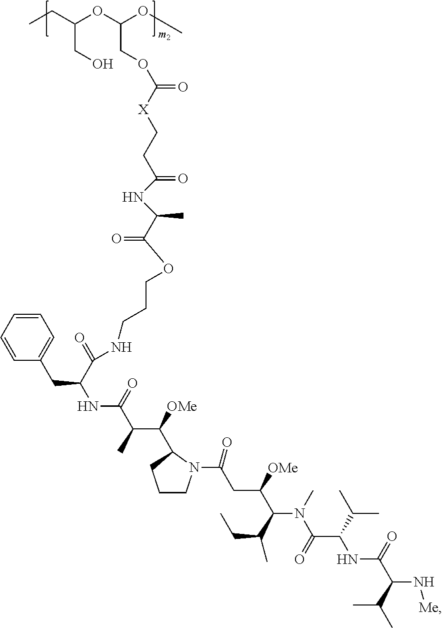

wherein:

m₃ₐ is an integer from 0 to about 17,

m_{3b} is an integer from 1 to about 8, and

the terminal

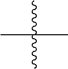

denotes the direct attachment of the one or more polymeric scaffolds to the HER2 antibody or antigen-binding fragment thereof having a molecular weight of 40 kDa or greater.

The scaffold of Formula (Id) or (Id-1) can include one or more of the following features:

The sum of m₃ₐ and m_{3b} is between 1 and 18.

When the PHF in Formula (Id) or (Id-1) has a molecular weight ranging from about 2 kDa to about 40 kDa, the sum of m, m₁, m₂, m₃ₐ and m_{3b} ranges from about 15 to about 300, m₁ is an integer from 1 to about 140, m₂ is an integer from 1 to about 40, m₃ₐ is an integer from 0 to about 17, m_{3b} is an integer from 1 to about 8, the sum of m₃ₐ and m_{3b} ranges from 1 and about 18, and the ratio between the PHF and the HER2 antibody or antigen-binding fragment thereof is 10 or less.

When the PHF in Formula (Id) or (Id-1) has a molecular weight ranging from about 2 kDa to about 20 kDa, the sum of m, m₁, m₂, m₃ₐ and m_{3b} ranges from about 15 to about 150, m₁ is an integer from 1 to about 70, m₂ is an integer from 1 to about 20, m₃ₐ is an integer from 0 to about 9, m_{3b} is an integer from 1 to about 8, the sum of m₃ₐ and m_{3b} ranges from 1 and about 10, and the ratio between the PHF and the HER2 antibody or antigen-binding fragment thereof is an integer from 2 to about 8.

When the PHF in Formula (Id) or (Id-1) has a molecular weight ranging from about 3 kDa to about 15 kDa, the sum of m, m₁, m₂, m₃ₐ and m_{3b} ranges from about 20 to about 110, m₁ is an integer from 2 to about 50, m₂ is an integer from 2 to about 15, m₃ₐ is an integer from 0 to about 7, m_{3b} is an integer from 1 to about 8, the sum of m₃ₐ and m_{3b} ranges from 1 and about 8; and the ratio between the PHF and the HER2 antibody or antigen-binding fragment thereof is an integer from 2 to about 8 (e.g., from about 2 to about 6 or from about 2 to about 4).

When the PHF in Formula (Id) or (Id-1) has a molecular weight ranging from about 5 kDa to about 10 kDa, the sum of m, m₁, m₂, m₃ₐ and m_{3b} ranges from about 40 to about 75, m₁ is an integer from about 2 to about 35, m₂ is an integer from about 2 to about 10, m₃ₐ is an integer from 0 to about 4, m_{3b} is an integer from 1 to about 5, the sum of m₃ₐ and m_{3b} ranges from 1 and about 5; and the ratio between the PHF and the HER2 antibody or antigen-binding fragment thereof is an integer from 2 to about 8 (e.g., from about 2 to about 6 or from about 2 to about 4).

In certain embodiments, the ratio between auristatin F hydroxypropyl amide (“AF HPA”) and the HER2 antibody or antigen-binding fragment thereof ranges from about 30:1 to about 6:1 (e.g., about 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1 or 6:1).

In certain embodiments, the ratio between AF HPA and the HER2 antibody or antigen-binding fragment thereof ranges from about 25:1 to about 6:1 (e.g., about 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1 or 6:1).

In other embodiments, the ratio between AF HPA and the HER2 antibody or antigen-binding fragment thereof ranges from about 20:1 to about 6:1 (e.g., about 20:1, 19:1, 18:1, 17:1, 16:1, 15:1, 14:1, 13:1, 12:1, 11:1, 10:1, 9:1, 8:1, 7:1 or 6:1).

In some embodiments, the ratio between AF HPA and HER2 antibody or antigen-binding fragment thereof ranges from about 16:1 to about 9:1 (e.g., about 16:1, 15:1, 14:1, 13:1, 12:1, 11:1, 10:1 or 9:1).

For example, the ratio between AF HPA and the HER2 antibody or antigen-binding fragment thereof ranges from about 15:1 to about 12:1 (e.g., about 15:1, 14:1, 13:1 or 12:1).

In some embodiments, the ratio between AF HPA and HER2 antibody or antigen-binding fragment thereof ranges from about 15:1 to about 11:1 (e.g., about 15:1, 14:1, 13:1, 12:1 or 11:1).

In some embodiments, the ratio between AF HPA and HER2 antibody or antigen-binding fragment thereof ranges from about 15:1 to about 10:1 (e.g., about 15:1, 14:1, 13:1, 12:1, 11:1 or 10:1).

In some embodiments, the ratio between AF HPA and HER2 antibody or antigen-binding fragment thereof can be about 12:1 to about 9:1 (e.g., about 12:1, 11:1, 10:1 or 9:1).

In certain embodiments, the ratio between monomethyl auristatin F hydroxypropyl amide (“MMAF HPA”) and the HER2 antibody or antigen-binding fragment thereof ranges from about 30:1 to about 6:1 (e.g., about 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1 or 6:1).

In certain embodiments, the ratio between MMAF HPA and the HER2 antibody or antigen-binding fragment thereof ranges from about 25:1 to about 6:1 (e.g., about 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1 or 6:1).

In other embodiments, the ratio between MMAF HPA and the HER2 antibody or antigen-binding fragment thereof ranges from about 20:1 to about 6:1 (e.g., about 20:1, 19:1, 18:1, 17:1, 16:1, 15:1, 14:1, 13:1, 12:1, 11:1, 10:1, 9:1, 8:1, 7:1 or 6:1).

In some embodiments, the ratio between MMAF HPA and HER2 antibody or antigen-binding fragment thereof ranges from about 16:1 to about 9:1 (e.g., about 16:1, 15:1, 14:1, 13:1, 12:1, 11:1, 10:1 or 9:1).

In some embodiments, the ratio between MMAF HPA and HER2 antibody or antigen-binding fragment thereof ranges from about 15:1 to about 9:1 (e.g., about 15:1, 14:1, 13:1, 12:1, 11:1, 10:1 or 9:1).

In some embodiments, the ratio between MMAF HPA and HER2 antibody or antigen-binding fragment thereof ranges from about 15:1 to about 12:1 (e.g., about 15:1, 14:1, 13:1 or 12:1).

In some embodiments, the ratio between MMAF HPA and HER2 antibody or antigen-binding fragment thereof ranges from about 15:1 to about 10:1 (e.g., about 15:1, 14:1, 13:1, 12:1, 11:1 or 10:1).

In some embodiments, the ratio between MMAF HPA and HER2 antibody or antigen-binding fragment thereof ranges from about 12:1 to about 9:1 (e.g., about 12:1, 11:1, 10:1 or 9:1).

In certain embodiments, the ratio between PHF and HER2 antibody or antigen-binding fragment thereof ranges from about 10:1 to about 1:1 (e.g., about 10:1, 9:1, 8:1, 7:1, 6:1, 5:1, 4:1, 3:1, 2:1 or 1:1).

In certain embodiments, the ratio between PHF and HER2 antibody or antigen-binding fragment thereof ranges from about 8:1 to about 2:1 (e.g., about 8:1, 7:1, 6:1, 5:1, 4:1, 3:1 or 2:1).

In other embodiments, the ratio between PHF and HER2 antibody or antigen-binding fragment thereof ranges from about 6:1 to about 1:1 (e.g., about 6:1, 5:1, 4:1, 3:1, 2:1 or 1:1).

In other embodiments, the ratio between PHF and HER2 antibody or antigen-binding fragment thereof ranges from about 6:1 to about 2:1 (e.g., about 6:1, 5:1, 4:1, 3:1 or 2:1).

In other embodiments, the ratio between PHF and HER2 antibody or antigen-binding fragment thereof ranges from about 5:1 to about 2:1 (e.g., about 5:1, 4:1, 3:1 or 2:1).

In other embodiments, the ratio between PHF and HER2 antibody or antigen-binding fragment thereof ranges from about 6:1 to about 3:1 (e.g., about 6:1, 5:1, 4:1 or 3:1).

In some embodiments, the ratio between PHF and HER2 antibody or antigen-binding fragment thereof ranges from about 5:1 to about 3:1 (e.g., about 5:1, 4:1 or 3:1).

In some embodiments, the ratio between PHF and HER2 antibody or antigen-binding fragment thereof ranges from about 4:1 to about 2:1 (e.g., about 4:1, 3:1 or 2:1).

The water-soluble maleimido blocking moieties (e.g., Xₐ or X_{b}) are moieties that can be covalently attached to one of the two olefin carbon atoms upon reaction of the maleimido group with a thiol-containing compound of Formula (II):

R₉₀—(CH₂)_{d}—SH (II)

wherein:

R₉₀ is NHR₉₁, OH, COOR₉₃, CH(NHR₉₁)COOR₉₃ or a substituted phenyl group;

R₉₃ is hydrogen or C₁₋₄ alkyl;

R₉₁ is hydrogen, CH₃ or CH₃CO and

d is an integer from 1 to 3.

In one embodiment, the water-soluble maleimido blocking compound of Formula (II) can be cysteine, N-acetyl cysteine, cysteine methyl ester, N-methyl cysteine, 2-mercaptoethanol, 3-mercaptopropanoic acid, 2-mercaptoacetic acid, mercaptomethanol (i.e., HOCH₂SH), benzyl thiol in which phenyl is substituted with one or more hydrophilic substituents, or 3-aminopropane-1-thiol. The one or more hydrophilic substituents on phenyl comprise OH, SH, methoxy, ethoxy, COOH, CHO, COC₁₋₄ alkyl, NH₂, F, cyano, SO₃H, PO₃H, and the like.

In another aspect, the water-soluble maleimido blocking group is —S—(CH₂)_{d}—R₉₀, in which,

R₉₀ is OH, COOH, or CH(NHR₉₁)COOR₉₃;

R₉₃ is hydrogen or CH₃;

R₉₁ is hydrogen or CH₃CO; and

d is 1 or 2.

In another embodiment, the water-soluble maleimido blocking group is —S—CH₂—CH(NH₂)COOH.

In certain embodiments, the conjugate described herein comprises one or more D-carrying PHF, each of which independently is of Formula (If), wherein the PHF has a molecular weight ranging from about 2 kDa to about 40 kDa:

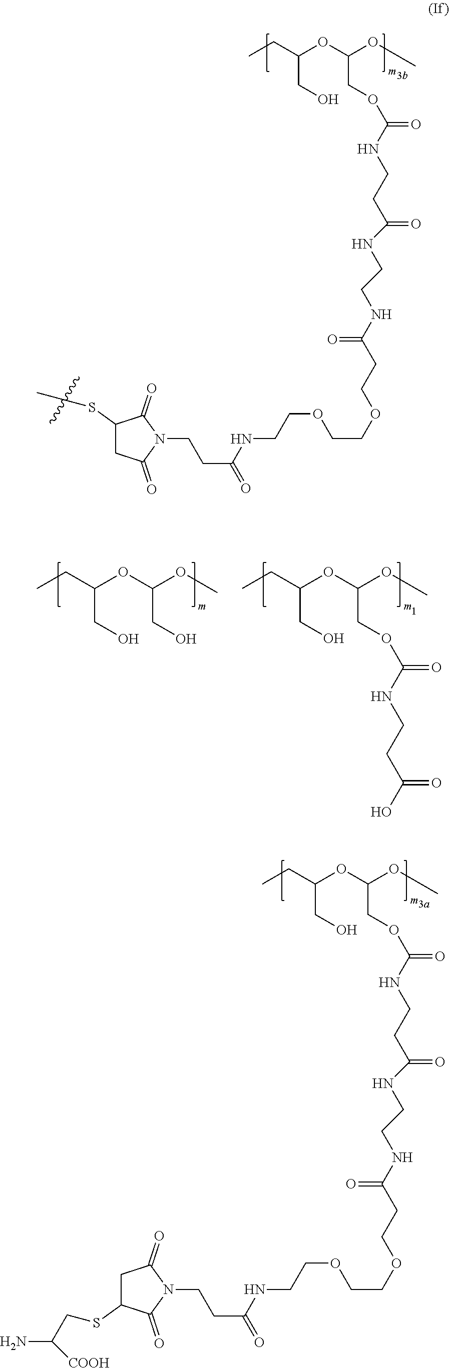

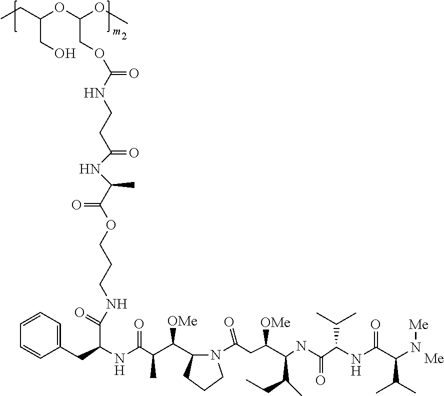

wherein:

m is an integer from 1 to about 300,

m₁ is an integer from 1 to about 140,

m₂ is an integer from 1 to about 40,

m₃ₐ is an integer from 0 to about 17,

m_{3b} is an integer from 1 to about 8;

the sum of m₃ₐ and m_{3b} ranges from 1 and about 18;

the sum of m, m₁, m₂, m₃ₐ, and m_{3b} ranges from about 15 to about 300;

the terminal

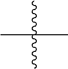

denotes the attachment of one or more PHF polymeric scaffolds to the antibody or antigen binding fragment thereof that specifically binds to an epitope of the human HER2 receptor and comprises a variable heavy chain complementarity determining region 1 (CDRH1) comprising the amino acid sequence FTFSSYSMN (SEQ ID NO: 25); a variable heavy chain complementarity determining region 2 (CDRH2) comprising the amino acid sequence YISSSSSTIYYADSVKG (SEQ ID NO: 26); a variable heavy chain complementarity determining region 3 (CDRH3) comprising the amino acid sequence GGHGYFDL (SEQ ID NO: 27); a variable light chain complementarity determining region 1 (CDRL1) comprising the amino acid sequence RASQSVSSSYLA (SEQ ID NO: 28); a variable light chain complementarity determining region 2 (CDRL2) comprising the amino acid sequence GASSRAT (SEQ ID NO: 21); and a variable light chain complementarity determining region 3 (CDRL3) comprising the amino acid sequence QQYHHSPLT (SEQ ID NO: 29); and

the ratio between the PHF and the antibody is 10 or less.

The scaffold of Formula (If) can include one or more of the following features:

When the PHF in Formula (If) has a molecular weight ranging from about 2 kDa to about 20 kDa, the sum of m, m₁, m₂, m₃ₐ and m_{3b} ranges from about 15 to about 150, m₁ is an integer from 1 to about 70, m₂ is an integer from 1 to about 20, m₃ₐ is an integer from 0 to about 9, m_{3b} is an integer from 1 to about 8, the sum of m₃ₐ and m_{3b} ranges from 1 and about 10, and the ratio between the PHF and the antibody is an integer from 2 to about 8.

When the PHF in Formula (If) has a molecular weight ranging from about 3 kDa to about 15 kDa, the sum of m, m₁, m₂, m₃ₐ and m_{3b} ranges from about 20 to about 110, m₁ is an integer from 2 to about 50, m₂ is an integer from 2 to about 15, m₃ₐ is an integer from 0 to about 7, m_{3b} is an integer from 1 to about 8, the sum of m₃ₐ and m_{3b} ranges from 1 and about 8; and the ratio between the PHF and the antibody is an integer from 2 to about 8 (e.g., from about 2 to about 6 or from about 2 to about 4).

When the PHF in Formula (If) has a molecular weight ranging from about 5 kDa to about 10 kDa, the sum of m, m₁, m₂, m₃ₐ and m_{3b} ranges from about 40 to about 75, m₁ is an integer from about 2 to about 35, m₂ is an integer from about 2 to about 10, m₃ₐ is an integer from 0 to about 4, m_{3b} is an integer from 1 to about 5, the sum of m₃ₐ and m_{3b} ranges from 1 and about 5; and the ratio between the PHF and the antibody is an integer from 2 to about 8 (e.g., from about 2 to about 6 or from about 2 to about 4).

In certain embodiments, the ratio between auristatin F hydroxypropyl amide (“AF HPA”) and the antibody ranges from about 30:1 to about 6:1 (e.g., about 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1 or 6:1).

In certain embodiments, the ratio between AF HPA and the antibody ranges from about 25:1 to about 6:1 (e.g., about 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1 or 6:1).

In other embodiments, the ratio between AF HPA and the antibody ranges from about 20:1 to about 6:1 (e.g., about 20:1, 19:1, 18:1, 17:1, 16:1, 15:1, 14:1, 13:1, 12:1, 11:1, 10:1, 9:1, 8:1, 7:1 or 6:1).

In some embodiments, the ratio between AF HPA and the antibody ranges from about 16:1 to about 10:1 (e.g., about 16:1, 15:1, 14:1, 13:1, 12:1, 11:1 or 10:1).

In some embodiments, the ratio between AF and the antibody ranges from about 15:1 to about 11:1 (e.g., about 15:1, 14:1, 13:1, 12:1 or 11:1).

In some embodiments, the ratio between AF HPA and the antibody ranges from about 15:1 to about 12:1 (e.g., about 15:1, 14:1, 13:1 or 12:1).

In some embodiments, the ratio between AF HPA and the antibody ranges from about 12:1 to about 9:1 (e.g., about 12:1, 11:1, 10:1 or 9:1).

In some embodiments, the ratio between AF HPA and the antibody ranges from about 12:1 to about 10:1 (e.g., about 12:1, 11:1 or 10:1).

In certain embodiments, the ratio between PHF and the antibody ranges from about 10:1 to about 1:1 (e.g., about 10:1, 9:1, 8:1, 7:1, 6:1, 5:1, 4:1, 3:1, 2:1 or 1:1).

In certain embodiments, the ratio between PHF and the antibody ranges from about 8:1 to about 2:1 (e.g., about 8:1, 7:1, 6:1, 5:1, 4:1, 3:1 or 2:1).

In other embodiments, the ratio between PHF and the antibody ranges from about 6:1 to about 1:1 (e.g., about 6:1, 5:1, 4:1, 3:1, 2:1 or 1:1).

In other embodiments, the ratio between PHF and the antibody ranges from about 6:1 to about 2:1 (e.g., about 6:1, 5:1, 4:1, 3:1 or 2:1).

In other embodiments, the ratio between PHF and the antibody ranges from about 6:1 to about 3:1 (e.g., about 6:1, 5:1, 4:1 or 3:1).

In other embodiments, the ratio between PHF and the antibody ranges from about 5:1 to about 2:1 (e.g., about 5:1, 4:1, 3:1 or 2:1).

In some embodiments, the ratio between PHF and the antibody ranges from about 5:1 to about 3:1 (e.g., about 5:1, 4:1 or 3:1).

In some embodiments, the ratio between PHF and the antibody ranges from about 4:1 to about 3:1 (e.g., about 4:1, 3:1 or 2:1).

In another aspect, the conjugate described herein is of Formula (Ib):

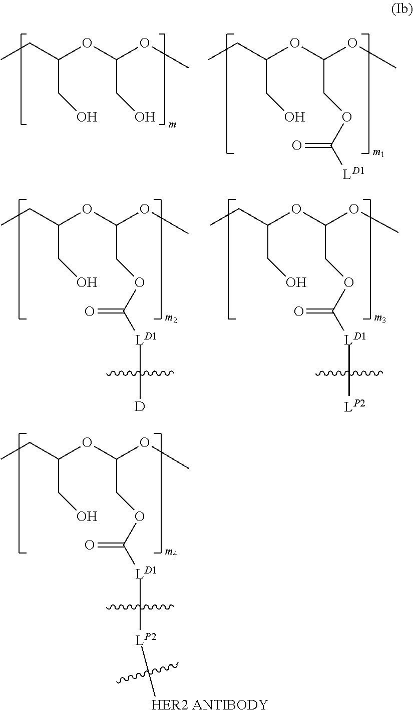

wherein:

HER2 ANTIBODY denotes the HER2 antibody or antigen-binding fragment thereof described herein;

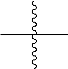

between L^{P2} and HER2 ANTIBODY denotes direct or indirect attachment of HER2 ANTIBODY to L^{P2},

each occurrence of HER2 ANTIBODY independently has a molecular weight of less than 200 kDa,

m is an integer from 1 to about 2200,

m₁ is an integer from 1 to about 660,

m₂ is an integer from 3 to about 300,

m₃ is an integer from 0 to about 110,

m₄ is an integer from 1 to about 60; and

the sum of m, m₁, m₂, m₃ and m₄ ranges from about 150 to about 2200.

In Formula (Ib), m₁ is an integer from about 10 to about 660 (e.g., about 10-250).

When the PHF in Formula (Ib) has a molecular weight ranging from about 50 kDa to about 100 kDa (i.e., the sum of m, m₁, m₂, m₃, and m₄ ranging from about 370 to about 740), m₂ is an integer from 5 to about 100, m₃ is an integer from 1 to about 40, m₄ is an integer from 1 to about 20, and/or m₁ is an integer from 1 to about 220 (e.g., m₁ being about 15-80).

In Formula (Ib), each HER2 ANTIBODY independently has a molecular weight of 120 kDa or less, 80 kDa or less, 70 kDa or less, 60 kDa or less, 50 kDa or less, 40 kDa or less, 30 kDa or less, 20 kDa or less or 10 kDa or less, or about 4 kDa to 80 kDa (e.g., 4-20 kDa, 20-30 kDa, or 30-70 kDa).

In the formulae for polymeric scaffolds disclosed herein, the disconnection or gap between the polyacetal units indicates that the units can be connected to each other in any order. In other words, the appending groups that contain, e.g., D, L^{P2}, and the antibody or antigen-binding fragment thereof, can be randomly distributed along the polymer backbone.

In some embodiments, the antibodies or antigen-binding fragment thereof disclosed herein comprise (1) heavy chain variable region CDRH1 comprising the amino acid sequence FTFSSYSMN (SEQ ID NO: 25); CDRH2 comprising the amino acid sequence YISSSSSTIYYADSVKG (SEQ ID NO: 26); CDRH3 comprising the amino acid sequence GGHGYFDL (SEQ ID NO: 27); and light chain variable region CDRL1 comprising the amino acid sequence RASQSVSSSYLA (SEQ ID NO: 28); CDRL2 comprising the amino acid sequence GASSRAT (SEQ ID NO: 21) and CDRL3 comprising the amino acid sequence QQYHHSPLT (SEQ ID NO: 29); (2) heavy chain variable region CDRH1 comprising the amino acid sequence FTFSGRSMN (SEQ ID NO: 30); CDRH2 comprising the amino acid sequence YISSDSRTIYYADSVKG (SEQ ID NO: 31); CDRH3 comprising the amino acid sequence GGHGYFDL (SEQ ID NO: 27); light chain variable region CDRL1 comprising the amino acid sequence RASQSVSSSYLA (SEQ ID NO: 28); CDRL2 comprising the amino acid sequence GASSRAT (SEQ ID NO: 21); and CDRL3 comprising the amino acid sequence QQYHHSPLT (SEQ ID NO: 29); (3) heavy chain variable region CDRH1 comprising the amino acid sequence FTFSSYGMH (SEQ ID NO: 17); CDRH2 comprising the amino acid sequence VIWYDGSNKYYADSVKG (SEQ ID NO: 18); CDRH3 comprising the amino acid sequence EAPYYAKDYMDV (SEQ ID NO: 19), and light chain variable region CDRL1 comprising the amino acid sequence RASQSVSSDYLA (SEQ ID NO: 20); CDRL2 comprising the amino acid sequence GASSRAT (SEQ ID NO: 21); and CDRL3 comprising the amino acid sequence QQYVSYWT (SEQ ID NO: 22); or (4) heavy chain variable region CDRH1 comprising the amino acid sequence FTFSSYGMH (SEQ ID NO: 17); CDRH2 comprising the amino acid sequence GIWWDGSNEKYADSVKG (SEQ ID NO: 23); CDRH3 comprising the amino acid sequence EAPYYAKDYMDV (SEQ ID NO: 19); and light chain variable region CDRL1 comprising the amino acid sequence RASQSVSSDYLA (SEQ ID NO: 20); CDRL2 comprising the amino acid sequence GASRRAT (SEQ ID NO: 24); and CDRL3 comprising the amino acid sequence QQYVSYWT (SEQ ID NO: 22).

In some embodiments, the HER2 antibodies or antigen binding fragments thereof disclosed herein specifically binds to an epitope of the human HER2 receptor that includes residues 452 to 531 of the extracellular domain of the human HER2 receptor, for example, residues 474 to 553 of SEQ ID NO: 38 or residues 452 to 531 of SEQ ID NO: 39.

In some embodiments, the HER2-targeted antibody-drug conjugates comprise an agent (e.g., D) conjugated to the HER2 antibody or fragment thereof disclosed herein directly or indirectly. In some embodiments, the agent is a therapeutic agent. In some embodiments, the agent is an antineoplastic agent. In some embodiments, the agent is a toxin or fragment thereof. In some embodiments the agent is (a) an auristatin compound; (b) a calicheamicin compound; (c) a duocarmycin compound; (d) SN38, (e) a pyrrolobenzodiazepine; (f) a vinca compound; (g) a tubulysin compound; (h) a non-natural camptothecin compound; (i) a maytansinoid compound; (j) a DNA binding drug; (k) a kinase inhibitor; (l) a MEK inhibitor; (m) a KSP inhibitor; (n) a topoisomerase inhibitor; and analogs thereof or analogues thereof. In some embodiments, the agent is an agent promoting immunogenic cell death (e.g., an anthracycline, an immunotoxin, doxorubicin, mitoxantrone, oxaliplatin, or bortezomib). More examples of agents that promote immunogenic cell death include those described in L. Galluzzi et al., Nature Reviews Immunology 17, 97-111, which is incorporated herein by reference in its entirety. In some embodiments, the agent is any of the toxins described herein. In some embodiments, the agent is conjugated to the HER2 antibody via a linker. In some embodiments, the linker is a cleavable linker. In some embodiments, the linker is a non-cleavable linker.

In some embodiments, the immune checkpoint inhibitor suitable for the combinations and methods of the disclosure is a monoclonal antibody, a humanized antibody, a fully human antibody, a fusion protein or a combination thereof.

In some embodiments, the immune checkpoint inhibitors inhibits a checkpoint protein that comprises CTLA-4, PDL1, PDL2, PD1, BTLA, HVEM, TIM3, GAL9, LAG3, VISTA, KIR, 2B4, CD160, CGEN-15049, CHK1, CHK2, A2aR, a B-7 family ligand, CD2, CD27, CD28, CD30, CD40, CD70, CD80, CD86, CD137, CD226, CD276, DR3, GITR, HAVCR2, HVEM, IDO1, IDO2, ICOS (inducible T cell costimulator), LAIR1, LIGHT, MARCO (macrophage receptor with collagenous structure), OX-40, SLAM, TIGHT, VTCN1 or a combination thereof.

In some embodiments, the immune checkpoint inhibitor interacts with a ligand of a checkpoint protein that comprises CTLA-4, PDL1, PDL2, PD1, BTLA, HVEM, TIM3, GAL9, LAG3, VISTA, KIR, 2B4, CD160, CGEN-15049, CHK1, CHK2, A2aR, a B-7 family ligand, CD2, CD27, CD28, CD30, CD40, CD70, CD80, CD86, CD137, CD226, CD276, DR3, GITR, HAVCR2, HVEM, IDO1, IDO2, ICOS (inducible T cell costimulator), LAIR1, LIGHT, MARCO (macrophage receptor with collagenous structure), OX-40, SLAM, TIGHT, VTCN1 or a combination thereof.

In some embodiments, the immune checkpoint inhibitor inhibits a checkpoint protein that comprises CTLA-4, PDL1, PD1 or a combination thereof.

In some embodiments, the immune checkpoint inhibitor comprises pembrolizumab (MK-3475), nivolumab (BMS-936558), pidilizumab (CT-011), AMP-224, MDX-1 105, durvalumab (MEDI4736), MPDL3280A, BMS-936559, IPH2101, TSR-042, TSR-022, ipilimumab, lirilumab, atezolizumab, avelumab, tremelimumab, or a combination thereof.

In some embodiments, the immune checkpoint inhibitor comprises nivolumab (BMS-936558), ipilimumab, pembrolizumab, atezolizumab, tremelimumab, durvalumab, avelumab, or a combination thereof.

In some embodiments, the HER2-targeted antibody-drug conjugate and the immune checkpoint inhibitor are formulated in the same formulation.

In some embodiments, the HER2-targeted antibody-drug conjugate and the immune checkpoint inhibitor are formulated in separate formulations.

In some aspects, the present disclosure provides a HER2-targeted antibody-drug conjugate disclosed herein for use in combination with (e.g., in temporal proximity with) an immunomodulatory therapy (e.g., an immuno-oncology agent such as an immune checkpoint inhibitor disclosed herein) in treating, preventing, delaying the progression of or otherwise ameliorating a symptom of one or more pathologies associated with aberrant HER2 expression, function and/or activation (e.g., a HER2 expressing tumor) or alleviating a symptom associated with such pathologies in a subject in need thereof.

In some aspects, the present disclosure provides a HER2-targeted antibody-drug conjugate disclosed herein for use in combination with (e.g., in temporal proximity with) an immunomodulatory therapy (e.g., an immuno-oncology agent such as an immune checkpoint inhibitor disclosed herein) in treating, preventing, delaying the progression of or otherwise ameliorating a symptom of one or more pathologies associated with HER2 expression, function and/or activation (e.g., a HER2 expressing tumor) or alleviating a symptom associated with such pathologies in a subject in need thereof.

In some aspects, the present disclosure provides an immunomodulatory therapy (e.g., an immuno-oncology agent such as an immune checkpoint inhibitor disclosed herein) for use in combination with (e.g., in temporal proximity with) a HER2-targeted antibody-drug conjugate disclosed herein in treating, preventing, delaying the progression of or otherwise ameliorating a symptom of one or more pathologies associated with aberrant HER2 expression, function and/or activation (e.g., a HER2 expressing tumor) or alleviating a symptom associated with such pathologies in a subject in need thereof.

In some aspects, the present disclosure provides an immunomodulatory therapy (e.g., an immuno-oncology agent such as an immune checkpoint inhibitor disclosed herein) for use in combination with (e.g., in temporal proximity with) a HER2-targeted antibody-drug conjugate disclosed herein in treating, preventing, delaying the progression of or otherwise ameliorating a symptom of one or more pathologies associated with HER2 expression, function and/or activation (e.g., a HER2 expressing tumor) or alleviating a symptom associated with such pathologies in a subject in need thereof.

In some aspects, the present disclosure provides a combination comprising a HER2-targeted antibody-drug conjugate and an immunomodulatory therapy (e.g., an immuno-oncology agent such as an immune checkpoint inhibitor disclosed herein) in treating, preventing, delaying the progression of or otherwise ameliorating a symptom of one or more pathologies associated with aberrant HER2 expression, function and/or activation (e.g., a HER2 expressing tumor) or alleviating a symptom associated with such pathologies in a subject in need thereof.

In some aspects, the present disclosure provides a combination comprising a HER2-targeted antibody-drug conjugate and an immunomodulatory therapy (e.g., an immuno-oncology agent such as an immune checkpoint inhibitor disclosed herein) in treating, preventing, delaying the progression of or otherwise ameliorating a symptom of one or more pathologies associated with HER2 expression, function and/or activation (e.g., a HER2 expressing tumor) or alleviating a symptom associated with such pathologies in a subject in need thereof.

In some aspects, the present disclosure provides use of a HER2-targeted antibody-drug conjugate disclosed herein in the manufacture of a medicament for use in combination with (e.g., in temporal proximity with) an immunomodulatory therapy (e.g., an immuno-oncology agent such as an immune checkpoint inhibitor disclosed herein) in treating, preventing, delaying the progression of or otherwise ameliorating a symptom of one or more pathologies associated with aberrant HER2 expression, function and/or activation (e.g., a HER2 expressing tumor) or alleviating a symptom associated with such pathologies in a subject in need thereof.

In some aspects, the present disclosure provides use of a HER2-targeted antibody-drug conjugate disclosed herein in the manufacture of a medicament for use in combination with (e.g., in temporal proximity with) an immunomodulatory therapy (e.g., an immuno-oncology agent such as an immune checkpoint inhibitor disclosed herein) in treating, preventing, delaying the progression of or otherwise ameliorating a symptom of one or more pathologies associated with HER2 expression, function and/or activation (e.g., a HER2 expressing tumor) or alleviating a symptom associated with such pathologies in a subject in need thereof.

In some aspects, the present disclosure provides use of an immunomodulatory therapy (e.g., an immuno-oncology agent such as an immune checkpoint inhibitor disclosed herein) in the manufacture of a medicament for use in combination with (e.g., in temporal proximity with) a HER2-targeted antibody-drug conjugate disclosed herein in treating, preventing, delaying the progression of or otherwise ameliorating a symptom of one or more pathologies associated with aberrant HER2 expression, function and/or activation (e.g., a HER2 expressing tumor) or alleviating a symptom associated with such pathologies in a subject in need thereof.

In some aspects, the present disclosure provides use of an immunomodulatory therapy (e.g., an immuno-oncology agent such as an immune checkpoint inhibitor disclosed herein) in the manufacture of a medicament for use in combination with (e.g., in temporal proximity with) a HER2-targeted antibody-drug conjugate disclosed herein in treating, preventing, delaying the progression of or otherwise ameliorating a symptom of one or more pathologies associated with HER2 expression, function and/or activation (e.g., a HER2 expressing tumor) or alleviating a symptom associated with such pathologies in a subject in need thereof.

In some aspects, the present disclosure provides use of a HER2-targeted antibody-drug conjugate and an immunomodulatory therapy (e.g., an immuno-oncology agent such as an immune checkpoint inhibitor disclosed herein) in the manufacture of a medicament for treating, preventing, delaying the progression of or otherwise ameliorating a symptom of one or more pathologies associated with aberrant HER2 expression, function and/or activation (e.g., a HER2 expressing tumor) or alleviating a symptom associated with such pathologies in a subject in need thereof.

In some aspects, the present disclosure provides use of a HER2-targeted antibody-drug conjugate and an immunomodulatory therapy (e.g., an immuno-oncology agent such as an immune checkpoint inhibitor disclosed herein) in the manufacture of a medicament for treating, preventing, delaying the progression of or otherwise ameliorating a symptom of one or more pathologies associated with HER2 expression, function and/or activation (e.g., a HER2 expressing tumor) or alleviating a symptom associated with such pathologies in a subject in need thereof.

In some aspects, the present disclosure provides methods of treating, preventing, delaying the progression of or otherwise ameliorating a symptom of one or more pathologies associated with aberrant HER2 expression, function and/or activation (e.g., a HER2 expressing tumor) or alleviating a symptom associated with such pathologies, by administering a combination comprising a HER2-targeted antibody-drug conjugate and an immunomodulatory therapy, e.g., an immuno-oncology agent such as an immune checkpoint inhibitor disclosed herein to a subject in which such treatment or prevention is desired. The subject to be treated is, e.g., human. The combination is administered in an amount sufficient to treat, prevent or alleviate a symptom associated with the pathology.

In some aspects, the present disclosure provides methods of treating, preventing, delaying the progression of or otherwise ameliorating a symptom of one or more pathologies associated with HER2 expression, function and/or activation (e.g., a HER2 expressing tumor) or alleviating a symptom associated with such pathologies, by administering a combination comprising a HER2-targeted antibody-drug conjugate and an immunomodulatory therapy, e.g., an immuno-oncology agent such as an immune checkpoint inhibitor disclosed herein to a subject in which such treatment or prevention is desired. The subject to be treated is, e.g., human. The combination is administered in an amount sufficient to treat, prevent or alleviate a symptom associated with the pathology.

In some embodiments, the HER2-targeted antibody-drug conjugate and the immunomodulatory therapy (e.g., the immune checkpoint inhibitor) are administered simultaneously.

In some embodiments, the HER2-targeted antibody-drug conjugate and the immunomodulatory therapy (e.g., the immune checkpoint inhibitor) are administered in temporal proximity.

In some embodiments, the HER2-targeted antibody-drug conjugate and the immunomodulatory therapy (e.g., the immune checkpoint inhibitor) are administered sequentially in either order or in alternation.

In some embodiments, the HER2-targeted antibody-drug conjugate is administered prior to the administration of the immunomodulatory therapy (e.g., the immune checkpoint inhibitor).

In some embodiments, immunomodulatory therapy (e.g., the immune checkpoint inhibitor) is administered prior to the administration of the HER2-targeted antibody-drug conjugate.

Pathologies treated and/or prevented using the combination therapies disclosed herein include, for example, a cancer. For example, the combination therapies disclosed herein are useful in treating, preventing, delaying the progression of or otherwise ameliorating a symptom of a cancer selected from the group consisting of anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, skin cancer, myeloma, oral cancer, rectal cancer, throat cancer, bladder cancer, breast cancer, uterine cancer, ovarian cancer, prostate cancer, lung cancer, non-small cell lung cancer (NSCLC), colon cancer, pancreatic cancer, renal cancer, and gastric cancer.

In some embodiments, the combination comprising a HER2-targeted antibody-drug conjugate and an immune checkpoint inhibitor disclosed herein are useful in treating, preventing, the delaying the progression of or otherwise ameliorating a symptom of breast cancer.

In some embodiments, the combination therapies disclosed herein are useful in treating, preventing, the delaying the progression of or otherwise ameliorating a symptom of gastric cancer.

In some embodiments, the combination therapies disclosed herein are useful in treating, preventing, the delaying the progression of or otherwise ameliorating a symptom of non-small cell lung cancer (NSCLC).

In some embodiments, the combination therapies disclosed herein are useful in treating, preventing, the delaying the progression of or otherwise ameliorating a symptom of ovarian cancer.

Also disclosed are kits comprising a HER2-targeted antibody-drug conjugate and an immune checkpoint inhibitor. The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

The disclosure also provides kits and/or methods for identifying or otherwise refining, e.g., stratifying, a patient population suitable for therapeutic administration of a HER2 antibody or antigen binding fragment thereof, conjugates thereof, and/or combination therapies thereof disclosed herein by identifying the HER2 score of subject prior to treatment with a HER2 antibody or antigen binding fragment thereof, conjugates thereof, and/or combination therapies thereof disclosed herein. In some embodiments, the subject is identified as having a scoring of 1+ or 2+ for HER2 expression. In some embodiments, the subject is identified as having a scoring of 1+ or 2+ for HER2 expression as detected by immunohistochemistry (IHC) analysis performed on a test cell population, and wherein the HER2 gene is not amplified in the test cell population. In some embodiments, the test cell population is derived from fresh, unfrozen tissue from a biopsy sample. In some embodiments, the test cell population is derived from a frozen tissue from a biopsy sample.

The IHC test measures the amount of HER2 receptor protein on the surface of cells in a cancer tissue sample, e.g., a breast cancer tissue sample or a gastric cancer sample, and assigns the detected level of cell surface HER2 receptor a HER2 score of 0, 1+, 2+ or 3+. If the subject's HER2 score is in the range of 0 to 1+, the cancer is deemed to be “HER2 negative.” If the score is 2+, the cancer is referred to as “borderline,” and a score of 3+ signifies that the cancer is “HER2 positive.”

In some embodiments, the subject is identified as having a scoring of 1+ or 2+ for HER2 expression and is refractory to chemotherapy, including standard, front-line chemotherapeutic agents. As used herein, the term subject includes humans and other mammals. In some embodiments, the subject is identified as having a scoring of 1+ or 2+ for HER2 expression and is suffering from breast cancer, gastric cancer, non-small cell lung cancer (NSCLC), or ovarian cancer.

In some embodiments, the combination comprising a HER2-targeted antibody-drug conjugate and an immune checkpoint inhibitor disclosed herein are useful in treating, preventing, the delaying the progression of or otherwise ameliorating a symptom of breast cancer in patients who have HER2 IHC 1+ or HER2 IHC 2+ without gene amplification, e.g., FISH- (or fluorescence in situ hybridization negative).

In some embodiments, the combination comprising a HER2-targeted antibody-drug conjugate and an immune checkpoint inhibitor disclosed herein are useful in treating, preventing, the delaying the progression of or otherwise ameliorating a symptom of breast cancer in patients who have advanced HER2 positive breast cancer and who have received prior treatment with Kadcyla (ado-trastuzumab emtansine).

In some embodiments, the combination comprising a HER2-targeted antibody-drug conjugate and an immune checkpoint inhibitor disclosed herein are useful in treating, preventing, the delaying the progression of or otherwise ameliorating a symptom of breast cancer in patients who have advanced HER2 positive breast cancer and who have not previously received prior treatment with Kadcyla (ado-trastuzumab emtansine).

In some embodiments, the combination comprising a HER2-targeted antibody-drug conjugate and an immune checkpoint inhibitor disclosed herein are useful in treating, preventing, the delaying the progression of or otherwise ameliorating a symptom of gastric cancer in patients who have HER2 IHC 1+ or HER2 IHC 2+ without gene amplification, e.g., FISH-.

In some embodiments, the combination comprising a HER2-targeted antibody-drug conjugate and an immune checkpoint inhibitor disclosed herein are useful in treating, preventing, the delaying the progression of or otherwise ameliorating a symptom of gastric cancer in patients who have advanced HER2 positive gastric cancer and who have received prior treatment with trastuzumab.

In some embodiments, the combination comprising a HER2-targeted antibody-drug conjugate and an immune checkpoint inhibitor disclosed herein are useful in treating, preventing, the delaying the progression of or otherwise ameliorating a symptom of gastric cancer in patients who have advanced HER2 positive gastric cancer and who have not previously received prior treatment with trastuzumab.

In some embodiments, the combination comprising a HER2-targeted antibody-drug conjugate and an immune checkpoint inhibitor disclosed herein are useful in treating, preventing, the delaying the progression of or otherwise ameliorating a symptom of non-small cell lung cancer (NSCLC) in patients who have HER2 IHC 2+, HER2 IHC 3+, any HER2 gene amplification or mutation status.

In some embodiments, the combination comprising a HER2-targeted antibody-drug conjugate and an immune checkpoint inhibitor disclosed herein are useful in treating, preventing, the delaying the progression of or otherwise ameliorating a symptom of non-small cell lung cancer (NSCLC) in patients who have HER2 IHC 1+ who have received prior platinum-based chemotherapy.

In some embodiments, the subject is refractory to chemotherapy, including standard, front-line chemotherapeutic agents. In some embodiments, the subject is resistant to treatment with Kadcyla (ado-trastuzumab emtansine).

A combination comprising a HER2-targeted antibody-drug conjugate and an immune checkpoint inhibitor used in any of the embodiments of the methods and uses provided herein can be administered at any stage of the disease. For example, such a combination therapy can be administered to a patient suffering cancer of any stage, from early to metastatic.

A combination therapy comprising a HER2-targeted antibody-drug conjugate and an immune checkpoint inhibitor used in any of the embodiments of these methods and uses can be administered either without another therapeutic agent, or in further combination with one or more chemotherapeutic agents or other agents. In some embodiments, the additional agent is any of the toxins described herein. In some embodiments, the additional agent is (1) HER2 inhibitors, (2) EGFR inhibitors (e.g., tyrosine kinase inhibitors or targeted anti-EGFR antibodies), (3) BRAF inhibitors, (4) ALK inhibitors, (5) hormone receptor inhibitors, (6) mTOR inhibitors, (7) VEGF inhibitors, or (8) cancer vaccines. In some embodiments, the additional agent is a standard, first line chemotherapeutic agent, such as, for example, trastuzumab, pertuzumab, ado-trastuzumab emtansine (Kadcyla), lapatinib, anastrozole, letrozole, exemestane, everolimus, fulvestrant, tamoxifen, toremifene, megestrol acetate, fluoxymesterone, ethinyl estradiol, paclitaxel, capecitabine, gemcitabine, eribulin, vinorelbine, cyclophosphamide, carboplatin, docetaxel, albumin-bound paclitaxel, cisplatin, epirubicin, ixabepilone, doxorubicin, fluorouracil, oxaliplatin, fluoropyrimidine, irinotecan, ramucirumab, mitomycin, leucovorin, cetuximab, bevacizumab, erlotinib, afatinib, crizotinib, permetrexed, ceritinib, etoposide, vinblastine, vincristine, ifosfamid, liposomal doxorubicin, topotecan, altretamine, melphalan or leuprolide acetate. In some embodiments, the additional agent is Kadcyla (ado-trastuzumab emtansine).

In some embodiments, the additional agent is at least a second antibody or antigen binding fragment thereof that specifically binds HER2. In some embodiments, the combination comprising a HER2-targeted antibody-drug conjugate and an immune checkpoint inhibitor of the disclosure is administered in combination with a HER2 antibody, a HER2 dimerization inhibitor antibody or a combination of a HER2 antibody and a HER2 dimerization inhibitor antibody, such as, for example, trastuzumab or pertuzumab or a combination thereof. In some embodiments, the combination comprising a HER2-targeted antibody-drug conjugate and an immune checkpoint inhibitor of the disclosure is administered in combination with a biosimilar of trastuzumab or a biosimilar of pertuzumab or a combination thereof.

These combinations of HER2-targeted antibody-drug conjugates and immune checkpoint inhibitors are useful in treating pathologies such as, for example, a cancer. For example, the combinations of combinations comprising HER2-targeted antibody-drug conjugates and immune checkpoint inhibitors disclosed herein in further combination with trastuzumab, pertuzumab or both trastuzumab and pertuzumab or a biosimilar of trastuzumab, a biosimilar of pertuzumab or both biosimilars, are useful in treating, preventing, delaying the progression of or otherwise ameliorating a symptom of a cancer (e.g., a cancer selected from the group consisting of anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, skin cancer, myeloma, oral cancer, rectal cancer, throat cancer, bladder cancer, breast cancer, uterine cancer, ovarian cancer, prostate cancer, lung cancer, non-small cell lung cancer (NSCLC), colon cancer, pancreatic cancer, renal cancer, and gastric cancer).

These combinations are also useful for increasing the degradation of HER2 when a HER2-expressing cell is contacted with these combinations. The level of HER2 degradation is detected using any art-recognized method for detecting HER2 degradation, including, but not limited to detecting levels of HER2 degradation in the presence and absence of a combination of HER2 antibodies (or biosimilars thereof). For example, the level of HER2 degradation is determined using western analysis of the lysates of HER2-expressing cells that have been treated with a combination of HER2 antibodies, as compared to the level of HER2 degradation in HER2-expressing cells that have not been treated with a combination of HER2 antibodies.

In some embodiments, the combinations comprising HER2-targeted antibody-drug conjugates and immune checkpoint inhibitors and additional agent(s) are formulated into a single therapeutic composition, and the components are administered simultaneously. Alternatively, the HER2-targeted antibody-drug conjugate, immune checkpoint inhibitor and additional agent, if any, are separate from each other, e.g., each is formulated into a separate therapeutic composition, and can be administered simultaneously, or at different times during a treatment regimen. For example, the antibody-drug conjugate and immune checkpoint inhibitor combination is administered prior to the administration of the additional agent; the antibody-drug conjugate and immune checkpoint inhibitor combination is administered subsequent to the administration of the additional agent; or the antibody-drug conjugate and immune checkpoint inhibitor combination and the additional agent are administered in an alternating fashion. As described herein, the antibody-drug conjugate and immune checkpoint inhibitor combination and additional agent are administered in single doses or in multiple doses.

Pharmaceutical compositions according to the disclosure can include an antibody, fragment thereof, conjugate thereof, and/or immune checkpoint inhibitor disclosed herein and a suitable carrier. These pharmaceutical compositions can be included in kits, such as, for example, diagnostic kits.

One skilled in the art will appreciate that the antibodies disclosed herein have a variety of uses. For example, the proteins disclosed herein are used as therapeutic agents. The antibodies disclosed herein are also used as reagents in diagnostic kits or as diagnostic tools, or these antibodies can be used in competition assays to generate therapeutic reagents.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTION OF THE FIGURES

FIG. 1 illustrates the anti-tumor efficacy of a combination of vehicle; XMT-1519 conjugate; pembrolizumab; and a combination of XMT-1519 conjugate and pembrolizumab in low passage TumorGraft™ model of non-small cell lung carcinoma in humanized mice (CTG-0860).

FIG. 2 shows ATP release from JIMT1 (panel (a)) and SKBR3 (panel (b)) cell lines after treatment with mitoxantrone, AF-HPA or XMT-1519 conjugate as compared to the control (untreated cells).

FIG. 3 shows calreticulin exposure on cell membrane in various cell lines after treatment with mitoxantrone (panel (a)), AF-HPA (panel (b)), or XMT-1519 conjugate (panel (c), (d) or (e)).

FIG. 4 shows the relative expression levels of HER2 in different human and mouse transgenic cell lines.

FIG. 5 shows the tumor response in mice after treatment with different regimens: (i) vehicle (ii) XMT-1519 conjugate (iii) Kadcyla, (iv) PD-1 (v) a combination of XMT-1519 conjugate and PD-1, and (vi) a combination of Kadcyla and PD-1.

FIG. 6 shows the tumor response in mice after treatment with different regimens: (i) vehicle (ii) a combination of XMT-1519 conjugate and PD-1 concurrently, (iii) XMT-1519 conjugate followed by PD-1 4 days later; (iv) PD-1 followed by XMT-1519 conjugate 4 days later; and (v) a combination of Kadcyla and PD-1 concurrently,

### DETAILED DESCRIPTION

The present disclosure provides combinations comprising HER2-targeted antibody-drug conjugates and immune checkpoint inhibitors, and to methods of using these combinations as therapeutics and/or diagnostics.

The present disclosure also provides kits for combinations of HER2-targeted antibody-drug conjugates and immune checkpoint inhibitors.

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the terms “HER2” (also known as ErbB-2, NEU, HER-2, and CD340), when used herein, refers to human epidermal growth factor receptor 2 (SwissProt P04626) and includes any variants, isoforms and species homologs of HER2 which are naturally expressed by cells, including tumor cells, or are expressed on cells transfected with the HER2 gene. Species homologs include rhesus monkey HER2 (macaca mulatta; Genbank accession No. GI:109114897). These terms are synonymous and may be used interchangeably.

As used herein, the term “HER2 antibody” or “anti-HER2 antibody” is an antibody which binds specifically to the antigen HER2.

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” “or directed against” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, dAb (domain antibody), single chain, F_{ab}, F_{ab′} and F_{(ab′)2} fragments, scFvs, and an F_{ab} expression library.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “monoclonal antibody” (mAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. mAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

The terms “fragment,” “antibody fragment,” “antigen-binding fragment,” and “antigen binding fragment” are used interchangeably herein, unless otherwise specified. For example, antibody fragments may contain the idiotypes to a protein antigen may be produced by techniques known in the art including, but not limited to: (i) an F_{(ab′)2} fragment (e.g., being produced by pepsin digestion of an antibody molecule); (ii) an F_{ab} fragment (e.g., being generated by reducing the disulfide bridges of an F_{(ab′)2} fragment); (iii) an F_{ab} fragment (e.g., being generated by the treatment of the antibody molecule with papain and a reducing agent); and (iv) Fᵥ fragments.

As used herein, the term “epitope” includes any protein determinant capable of specific binding to an immunoglobulin or fragment thereof, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 μM; e.g., ≤100 nM, preferably ≤10 nM and more preferably ≤1 nM.

When used herein in the context of two or more antibodies, the term “competes with” or “cross-competes with” indicates that the two or more antibodies compete for binding to HER2, e.g., compete for HER2 binding in the assay described in U.S. Pat. No. 9,738,720, Examples 5 or 8. An antibody “blocks” or “cross-blocks” one or more other antibodies from binding to HER2 if the antibody competes with the one or more other antibodies 25% or more, with 25%-74% representing “partial block” and 75%-400% representing “full block”, preferably as determined using the assay described in U.S. Pat. No. 9,738,720, Examples 5 and 8. For some pairs of antibodies, competition or blocking in the assay described in U.S. Pat. No. 9,738,720 Examples 5 or 8 is only observed when one antibody is coated on the plate and the other is used to compete, and not vice versa. Unless otherwise defined or negated by context, the terms “competes with”, “cross-competes with”, “blocks” or “cross-blocks” when used herein is also intended to cover such pairs of antibodies.

As used herein an antibody which “inhibits HER dimerization” shall mean an antibody which inhibits, or interferes with, formation of a HER dimer. Preferably, such an antibody binds to HER2 at the heterodimeric binding site thereof. In one embodiment the dimerization inhibiting antibody herein is pertuzumab or MAb 2C4. Other examples of antibodies which inhibit HER dimerization include antibodies which bind to EGFR and inhibit dimerization thereof with one or more other HER receptors, such as, for example EGFR monoclonal antibody 806, MAb 806, which binds to activated or “untethered” EGFR (see Johns et al., J. Biol. Chem. 279(29):30375-30384 (2004)); antibodies which bind to HER3 and inhibit dimerization thereof with one or more other HER receptors; and antibodies which bind to HER4 and inhibit dimerization thereof with one or more other HER receptors.

The term “HER2 dimerization inhibitor” as used herein shall mean an agent that inhibits formation of a dimer or heterodimer comprising HER2.

As used herein, the term “internalization”, when used in the context of a HER2 antibody includes any mechanism by which the antibody is internalized into a HER2-expressing cell from the cell-surface and/or from surrounding medium, e.g., via endocytosis.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present disclosure is said to specifically bind to HER2, when the equilibrium dissociation constant (K_{d} or K_{D}) is ≤1 μM, preferably ≤100 nM, more preferably ≤10 nM, and most preferably ≤100 pM to about 1 pM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the disclosure include the nucleic acid sequences of SEQ ID NOs: 34 and 36, as well as nucleic acid molecules encoding the heavy chain immunoglobulin molecules presented in SEQ ID NOs: 1, 3, 5, and 7, and the nucleic acid sequences of SEQ ID NOs: 35 and 37, as well as nucleic acid molecules encoding the light chain immunoglobulin molecules represented in SEQ ID NOs: 2, 4, 6, and 8.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the disclosure comprise the heavy chain immunoglobulin molecules represented in SEQ ID NOs: 1, 3, 5, and 7, and the light chain immunoglobulin molecules represented in SEQ ID NOs: 2, 4, 6, and 8 as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxyribonucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g., for probes, although oligonucleotides may be double stranded, e.g., for use in the construction of a gene mutant. Oligonucleotides disclosed herein are either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes Oligonucleotides linkages such as phosphorothioate, phosphorodithioate, phosphoroselerloate, phosphorodiselenoate, phosphoroanilothioate, phoshoraniladate, phosphoronmidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). An oligonucleotide can include a label for detection, if desired.

The term “selectively hybridize” referred to herein means to detectably and specifically bind. Polynucleotides, oligonucleotides and fragments thereof in accordance with the disclosure selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments disclosed herein and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland7 Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4 hydroxyproline, y-carboxyglutamate, ε-N,N,N-trimethyllysine, ε-N-acetyllysine, O-phosphoserine, N-acetylserine, N-formylmethionine, 3-methylhistidine, 5-hydroxylysine, o-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the disclosure.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence. Fragments typically are at least 5, 6, 8 or 10 amino acids long, preferably at least 14 amino acids long′ more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has specific binding to HER2 under suitable binding conditions. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TINS p. 392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH—(cis and trans), —COCH₂—, CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The use of the articles “a”, “an”, and “the” in both the following description and claims are to be construed to cover both the singular and the plural, unless otherwise indicated herein or clearly contradicted by context. The terms “comprising”, “having”, “being of” as in “being of a chemical formula”, “including”, and “containing” are to be construed as open terms (i.e., meaning “including but not limited to”) unless otherwise noted. For example, a polymeric scaffold of a certain formula includes all the monomer units shown in the formula and may also include additional monomer units not shown in the formula. Additionally whenever “comprising” or another open-ended term is used in an embodiment, it is to be understood that the same embodiment can be more narrowly claimed using the intermediate term “consisting essentially of” or the closed term “consisting of.”

The term “about”, “approximately”, or “approximate”, when used in connection with a numerical value, means that a collection or range of values is included. For example, “about X” includes a range of values that are ±20%, +10%, +5%, +2%, +1%, +0.5%, +0.2%, or +0.1% of X, where X is a numerical value. In one embodiment, the term “about” refers to a range of values which are 5% more or less than the specified value. In another embodiment, the term “about” refers to a range of values which are 2% more or less than the specified value. In another embodiment, the term “about” refers to a range of values which are 1% more or less than the specified value.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “x being an integer between 1 and 6” and “x being an integer of 1 to 6” both mean “x being 1, 2, 3, 4, 5, or 6”, i.e., the terms “between X and Y” and “range from X to Y, are inclusive of X and Y and the integers there between.

All methods described herein can be performed in any suitable order unless otherwise indicated herein or otherwise clearly contradicted by context. The use of any and all examples, or exemplary language (e.g., “such as”) provided herein, is intended merely to better illustrate the invention and is not to be construed as a limitation on the scope of the claims unless explicitly otherwise claimed. No language in the specification is to be construed as indicating that any non-claimed element is essential to what is claimed.

“Protein based recognition-molecule” or “PBRM” refers to a molecule that recognizes and binds to a cell surface marker or receptor such as, a transmembrane protein, surface immobilized protein, or proteoglycan. Examples of PBRMs include but are not limited to, the XMT 1517 antibody, the XMT 1518 antibody, the XMT 1519 antibody and the XMT 1520 antibody described herein, as we all as other antibodies (e.g., Trastuzumab, Cetuximab, Rituximab, Bevacizumab, Epratuzumab, Veltuzumab, Labetuzumab, B7-H4, B7-H3, CA125, CD33, CXCR2, EGFR, FGFR1, FGFR2, FGFR3, FGFR4, HER2, NaPi2b, c-Met, MUC-1, NOTCH1, NOTCH2, NOTCH3, NOTCH4, PD-L1-and-anti-5T4), and antibodies or antigen binding fragments thereof described herein) or peptides (LHRH receptor targeting peptides, EC-1 peptide), lipocalins, such as, for example, anticalins, proteins such as, for example, interferons, lymphokines, growth factors, colony stimulating factors, and the like, peptides or peptide mimics, and the like. The protein based recognition molecule, in addition to targeting the modified polymer conjugate to a specific cell, tissue or location, may also have certain therapeutic effect such as antiproliferative (cytostatic and/or cytotoxic) activity against a target cell or pathway. The protein based recognition molecule comprises or may be engineered to comprise at least one chemically reactive group such as, —COOH, primary amine, secondary amine —NHR, —SH, or a chemically reactive amino acid moiety or side chains such as, for example, tyrosine, histidine, cysteine, or lysine.

“Biocompatible” as used herein is intended to describe compounds that exert minimal destructive or host response effects while in contact with body fluids or living cells or tissues. Thus a biocompatible group, as used herein, refers to an aliphatic, cycloalkyl, heteroaliphatic, heterocycloalkyl, aryl, or heteroaryl moiety, which falls within the definition of the term biocompatible, as defined above and herein. The term “Biocompatibility” as used herein, is also taken to mean that the compounds exhibit minimal interactions with recognition proteins, e.g., naturally occurring antibodies, cell proteins, cells and other components of biological systems, unless such interactions are specifically desirable. Thus, substances and functional groups specifically intended to cause the above minimal interactions, e.g., drugs and prodrugs, are considered to be biocompatible. Preferably (with exception of compounds intended to be cytotoxic, such as, e.g., antineoplastic agents), compounds are “biocompatible” if their addition to normal cells in vitro, at concentrations similar to the intended systemic in vivo concentrations, results in less than or equal to 1% cell death during the time equivalent to the half-life of the compound in vivo (e.g., the period of time required for 50% of the compound administered in vivo to be eliminated/cleared), and their administration in vivo induces minimal and medically acceptable inflammation, foreign body reaction, immunotoxicity, chemical toxicity and/or other such adverse effects. In the above sentence, the term “normal cells” refers to cells that are not intended to be destroyed or otherwise significantly affected by the compound being tested.

“Biodegradable”: As used herein, “biodegradable” polymers are polymers that are susceptible to biological processing in vivo. As used herein, “biodegradable” compounds or moieties are those that, when taken up by cells, can be broken down by the lysosomal or other chemical machinery or by hydrolysis into components that the cells can either reuse or dispose of without significant toxic effect on the cells. The term “biocleavable” as used herein has the same meaning of “biodegradable”. The degradation fragments preferably induce little or no organ or cell overload or pathological processes caused by such overload or other adverse effects in vivo. Examples of biodegradation processes include enzymatic and non-enzymatic hydrolysis, oxidation and reduction. Suitable conditions for non-enzymatic hydrolysis of the biodegradable protein-polymer-drug conjugates (or their components, e.g., the biodegradable polymeric carrier and the linkers between the carrier and the antibody or the drug molecule) described herein, for example, include exposure of the biodegradable conjugates to water at a temperature and a pH of lysosomal intracellular compartment. Biodegradation of some protein-polymer-drug conjugates (or their components, e.g., the biodegradable polymeric carrier and the linkers between the carrier and the antibody or the drug molecule), can also be enhanced extracellularly, e.g., in low pH regions of the animal body, e.g., an inflamed area, in the close vicinity of activated macrophages or other cells releasing degradation facilitating factors. In certain preferred embodiments, the effective size of the polymer carrier at pH˜7.5 does not detectably change over 1 to 7 days, and remains within 50% of the original polymer size for at least several weeks. At pH˜5, on the other hand, the polymer carrier preferably detectably degrades over 1 to 5 days, and is completely transformed into low molecular weight fragments within a two-week to several-month time frame. Polymer integrity in such tests can be measured, for example, by size exclusion HPLC. Although faster degradation may be in some cases preferable, in general it may be more desirable that the polymer degrades in cells with the rate that does not exceed the rate of metabolization or excretion of polymer fragments by the cells. In preferred embodiments, the polymers and polymer biodegradation byproducts are biocompatible.

“Maleimido blocking compound”: as used herein refers to a compound that can react with maleimide to convert it to succinimide and “maleimido blocking moiety” refers to the chemical moiety attached to the succinimide upon conversion. In certain embodiments, the maleimido blocking compound is a compound having a terminal thiol group for reacting with the maleimide. In one embodiment, the maleimido blocking compound is cysteine, N-acetyl cysteine, cysteine methyl ester, N-methyl cysteine, 2-mercaptoethanol, 3-mercaptopropanoic acid, 2-mercaptoacetic acid, mercaptomethanol (i.e., HOCH₂SH), benzyl thiol in which phenyl is substituted with one or more hydrophilic substituents, or 3-aminopropane-1-thiol.

“Hydrophilic”: The term “hydrophilic” as it relates to substituents, e.g., on the polymer monomeric units or on a maleimido blocking moiety to render them hydrophilic or water soluble, does not essentially differ from the common meaning of this term in the art, and denotes chemical moieties which contain ionizable, polar, or polarizable atoms, or which otherwise may be solvated by water molecules. Thus a hydrophilic group, as used herein, refers to an aliphatic, cycloalkyl, heteroaliphatic, heterocycloalkyl, aryl or heteroaryl moiety, which falls within the definition of the term hydrophilic, as defined above. Examples of particular hydrophilic organic moieties which are suitable include, without limitation, aliphatic or heteroaliphatic groups comprising a chain of atoms in a range of between about one and twelve atoms, hydroxyl, hydroxyalkyl, amine, carboxyl, amide, carboxylic ester, thioester, aldehyde, nitryl, isonitryl, nitroso, hydroxylamine, mercaptoalkyl, heterocycle, carbamates, carboxylic acids and their salts, sulfonic acids and their salts, sulfonic acid esters, phosphoric acids and their salts, phosphate esters, polyglycol ethers, polyamines, polycarboxylates, polyesters and polythioesters. In certain embodiments, hydrophilic substituents comprise a carboxyl group (COOH), an aldehyde group (CHO), a ketone group (COC₁₋₄ alkyl), a methylol (CH₂OH) or a glycol (for example, CHOH—CH₂OH or CH—(CH₂OH)₂), NH₂, F, cyano, SO₃H, PO₃H, and the like.

The term “hydrophilic” as it relates to the polymers disclosed herein generally does not differ from usage of this term in the art, and denotes polymers comprising hydrophilic functional groups as defined above. In a preferred embodiment, hydrophilic polymer is a water-soluble polymer. Hydrophilicity of the polymer can be directly measured through determination of hydration energy, or determined through investigation between two liquid phases, or by chromatography on solid phases with known hydrophobicity, such as, for example, C₄ or C18.

“Polymeric Carrier”: The term polymeric carrier, as used herein, refers to a polymer or a modified polymer, which is suitable for covalently attaching to or can be covalently attached to one or more drug molecules with a designated linker and/or one or more PBRMs with a designated linker.

“Physiological conditions”: The phrase “physiological conditions”, as used herein, relates to the range of chemical (e.g., pH, ionic strength) and biochemical (e.g., enzyme concentrations) conditions likely to be encountered in the extracellular fluids of living tissues. For most normal tissues, the physiological pH ranges from about 7.0 to 7.4. Circulating blood plasma and normal interstitial liquid represent typical examples of normal physiological conditions.

“Drug”: As used herein, the term “drug” refers to a compound which is biologically active and provides a desired physiological effect following administration to a subject in need thereof (e.g., an active pharmaceutical ingredient).

“Cytotoxic”: As used herein the term “cytotoxic” means toxic to cells or a selected cell population (e.g., cancer cells). The toxic effect may result in cell death and/or lysis. In certain instances, the toxic effect may be a sublethal destructive effect on the cell, e.g., slowing or arresting cell growth. In order to achieve a cytotoxic effect, the drug or prodrug may be selected from a group consisting of a DNA damaging agent, a microtubule disrupting agent, or a cytotoxic protein or polypeptide, amongst others.

“Cytostatic”: As used herein the term “cytostatic” refers to a drug or other compound which inhibits or stops cell growth and/or multiplication.

“Small molecule”: As used herein, the term “small molecule” refers to molecules, whether naturally-occurring or artificially created (e.g., via chemical synthesis) that have a relatively low molecular weight. Preferred small molecules are biologically active in that they produce a local or systemic effect in animals, preferably mammals, more preferably humans. In certain preferred embodiments, the small molecule is a drug and the small molecule is referred to as “drug molecule” or “drug” or “therapeutic agent”. In certain embodiments, the drug molecule has MW less than or equal to about 5 kDa. In other embodiments, the drug molecule has MW less than or equal to about 1.5 kDa. In embodiments, the drug molecule is selected from vinca alkaloids, auristatins, duocarmycins, tubulysins, non-natural camptothecin compounds, topoisomerase inhibitors, DNA binding drugs, kinase inhibitors, MEK inhibitors, KSP inhibitors, calicheamicins, SN38, pyrrolobenzodiazepines, and analogs thereof. Preferably, though not necessarily, the drug is one that has already been deemed safe and effective for use by an appropriate governmental agency or body, e.g., the FDA. For example, drugs for human use listed by the FDA under 21 C.F.R. §§ 330.5, 331 through 361, and 440 through 460; drugs for veterinary use listed by the FDA under 21 C.F.R. §§ 500 through 589, incorporated herein by reference, are all considered suitable for use with the present hydrophilic polymers.

“Drug derivative” or “modified drug” or the like as used herein, refers to a compound that comprises the drug molecule intended to be delivered by the conjugate disclosed herein and a functional group capable of attaching the drug molecule to the polymeric carrier.

“Active form” as used herein refers to a form of a compound that exhibits intended pharmaceutical efficacy in vivo or in vitro. In particular, when a drug molecule intended to be delivered by the conjugate disclosed herein is released from the conjugate, the active form can be the drug itself or its derivatives, which exhibit the intended therapeutic properties. The release of the drug from the conjugate can be achieved by cleavage of a biodegradable bond of the linker which attaches the drug to the polymeric carrier. The active drug derivatives accordingly can comprise a portion of the linker.

“PHF” refers to poly(1-hydroxymethylethylene hydroxymethyl-formal).

As used herein, the terms “polymer unit”, “monomeric unit”, “monomer”, “monomer unit”, “unit” all refer to a repeatable structural unit in a polymer.

As used herein, “molecular weight” or “MW” of a polymer or polymeric carrier/scaffold or polymer conjugates refers to the weight average molecular weight of the unmodified polymer unless otherwise specified.

“Immune checkpoint inhibitor” or “immune checkpoint inhibiting agent” or “immune checkpoint blocking agent” or “immune checkpoint modulator” as used herein, refers to an agent that binds an inhibitory immune checkpoint protein and blocks its activity thereby enabling the immune system to recognize tumor cells and allowing a sustained immunotherapy response. The inhibition can be competitive or non-competitive inhibition that can be steric or allosteric. In cases where an immune checkpoint protein is an immune stimulating protein, an immune checkpoint inhibitor acts to promote the activity of the immune stimulating protein, such as by binding and activating the stimulatory immune checkpoint protein or by inhibiting by interfering with, such as by binding or deactivating, inhibitors of the stimulatory immune checkpoint protein. An example of an immune checkpoint inhibitor is an anti-immune checkpoint protein antibody.

“Immune checkpoints” as used herein refer to inhibitory pathways of the immune system that are responsible for maintaining self-tolerance and modulating the duration and amplitude of physiological immune responses in peripheral tissues in order to minimize collateral tissue damage. Immune checkpoints are regulated by immune checkpoint proteins.

“Immune checkpoint protein” as used herein, refers to is a protein, typically a receptor (e.g., CTLA4 or PD-1) or a ligand (e.g., PD-L1) that regulates or modulates the extent of an immune response. The immune checkpoint proteins can be inhibitory or stimulatory. In particular, the immune checkpoint proteins are inhibitory to the activation of the immune response. Thus, inhibition of an inhibitory immune checkpoint protein acts to stimulate or activate an immune response, such as T cell activation and proliferation.

A “target” of an immune checkpoint inhibitor as used herein, is the immune checkpoint protein to which the immune checkpoint inhibitor or immune checkpoint inhibiting agent binds to block activity. Typically, the immune checkpoint inhibitor specifically binds to the target. For example, the target of the exemplary anti-CTLA4 antibody designated ipilimumab is CTLA4.

“Combination Therapy” as used herein refers to a treatment in which a subject is given two or more therapeutic agents, such as at least two or at least three therapeutic agents, for treating a single disease. For purposes herein, combination therapy includes therapy with a HER2-targeted antibody-drug conjugate and an immune checkpoint inhibitor.

As used herein “co-administration”, “co-administering” or “co-administered” refers to the administration of at least two different therapeutic agents sufficiently close in time. Such administration may be done in any order, including simultaneous administration, as well as temporally spaced order from a few seconds up to several days apart. Such administration may also include more than a single administration of one agent and/or independently the other agent. The administration of the agents may be by the same or different routes.

As used herein, “anti-CTLA4 antibody” refers to any antibody that specifically binds to cytotoxic T-lymphocyte-associated protein 4 (CTLA4) or a soluble fragment thereof and blocks the binding of ligands to CTLA4, thereby resulting in competitive inhibition of CTLA4 and inhibition of CTLA4-mediated inhibition of T cell activation. Hence, anti-CTLA4 antibodies are CTLA4 inhibitors. Reference to anti-CTLA4 antibodies herein include a full-length antibody and derivatives thereof, such as antigen-binding fragments thereof that specifically bind to CTLA4. Exemplary anti-CTLA4 antibodies include, but are not limited to, ipilimumab or tremelimumab, or a derivative or antigen-binding fragment thereof.

As used herein, a “cytotoxic T-lymphocyte-associated protein 4” (CTLA4; also known as CD 152) antigen refers to an inhibitory receptor of the immunoglobulin superfamily, that is bound by ligands such as CD80 (also called B7-1) and CD86, (also called B7-2). CTLA4 includes human and non-human proteins. In particular, CTLA4 antigen includes human CTLA4, which has the sequence of amino acids set forth in e.g., GenBank Accession No. AAL07473.1.

As used herein, “anti-PD-1 antibody” refers to any antibody that specifically binds to programmed cell death protein 1 (PD-1) or a soluble fragment thereof and blocks the binding of ligands to PD-1, thereby resulting in competitive inhibition of PD-1 and inhibition of PD-1 mediated inhibition of T cell activation. Hence, anti-PD-1 antibodies are PD-1 inhibitors. Reference to anti-PD-1 antibodies herein include a full-length antibody and derivatives thereof, such as antigen-binding fragments thereof that specifically bind to PD-1. Exemplary anti-PD-1 antibodies include, but are not limited to, nivolumab, MK-3475, pidilizumab, or a derivative or antigen-binding fragment thereof.

As used herein, a “programmed cell death protein 1” (PD-1) antigen refers to an inhibitory receptor, that is a type 1 membrane protein and is bound by ligands such as PD-L1 and PD-L2, which are members of the B7 family. PD-1 includes human and non-human proteins. In particular, PD-1 antigen includes human PD-1, which has the sequence of amino acids set forth in e.g., UniProt Accession No. Q15116.3. As used herein, anti-PD-L1 antibody refers to an antibody that specifically binds to programed death-ligand 1 (PD-L1) or a soluble fragment thereof and blocking the binding of the ligand to PD-1, thereby resulting in competitive inhibition of PD-1 and inhibition of PD-1 mediated inhibition of T cell activity. Hence, anti-PD-LI antibodies are PD-1 inhibitors. Reference to anti-PD-L1 antibodies herein include a full-length antibody and derivatives thereof, such as antigen-binding fragments thereof that specifically bind to PD-L1. Exemplary anti-PD-L1 antibodies include, but are not limited to, BMS-936559, MPDL3280A, MEDI4736 or a derivative or antigen-binding fragment thereof.

As used herein, “dosing regimen” or “dosage regimen” refers to the amount of agent, for example, the composition containing an HER2-targeted antibody-drug conjugate, administered, and the frequency of administration. The dosing regimen is a function of the disease or condition to be treated, and thus can vary.

As used herein, “frequency” of administration refers to the time between successive administrations of treatment. For example, frequency can be days, weeks or months. For example, frequency can be more than once weekly, for example, twice a week, three times a week, four times a week, five times a week, six times a week or: daily. Frequency also can be one, two, three or four weeks. The particular frequency is a function of the particular disease or condition treated. Generally, frequency is more than once weekly, and generally is twice weekly.

As used herein, a “cycle of administration” refers to the repeated schedule of the dosing regimen of administration of the enzyme and/or a second agent that is repeated over successive administrations. For example, an exemplary cycle of administration is a 28 day cycle with administration twice weekly for three weeks, followed by one-week of discontinued dosing.

As used herein, when referencing dosage based on mg/kg of the subject, an average human subject is considered to have a mass of about 70 kg-75 kg, such as 70 kg and a body surface area (BSA) of 1.73 m. As used herein, amelioration of the symptoms of a particular disease or disorder by a treatment, such as by administration of a pharmaceutical composition or other therapeutic, refers to any lessening, whether permanent or temporary, lasting or transient, of the symptoms or, adverse effects of a condition, such as, for example, reduction of adverse effects associated with or that occur upon administration of an HER2-targeted antibody-drug conjugate.

As used herein, “treating” or “treat” describes the management and care of a patient for the purpose of combating a disease, condition, or disorder and includes the administration of a conjugate of the disclosure, or a pharmaceutical composition thereof in combination with an immunomodulatory therapy, e.g., an immuno-oncology agent such as an immune checkpoint inhibitor, to alleviate the symptoms or complications of a disease, condition or disorder, or to eliminate the disease, condition or disorder.

As used herein, “prevention” or “prophylaxis” refers to reduction in the risk of developing a disease or condition, or reduction or elimination of the onset of the symptoms or complications of the disease, condition or disorder.

The term “effective amount” or “sufficient amount”, as it refers to an active agent, refers to the amount necessary to elicit the desired biological response. As used herein, a “therapeutically effective amount” or a “therapeutically effective dose” refers to an amount or quantity of an agent, compound, material, or composition containing a compound that is at least sufficient to produce a detectable therapeutic effect. The effect can be detected by any assay method known in the art. The precise effective amount for a subject will depend upon the subject's body weight, size, and health; the nature and extent of the condition; and the therapeutic selected for administration.

A “subject” includes a mammal. The mammal can be e.g., any mammal, e.g., a human, primate, bird, mouse, rat, fowl, dog, cat, cow, horse, goat, camel, sheep or a pig. Preferably, the mammal is a human.

As used herein, “unit dose form” or “unit dosage form” refers to physically discrete units suitable for human and animal subjects and packaged individually as is known in the art.

As used herein, a single dosage formulation refers to a formulation as a single dose.

As used herein, “temporal proximity” refers to that administration of one therapeutic agent (e.g., a HER2-targeted antibody-drug conjugate disclosed herein) occurs within a time period before or after the administration of another therapeutic agent (e.g., an immune checkpoint inhibitor disclosed herein), such that the therapeutic effect of the one therapeutic agent overlaps with the therapeutic effect of the another therapeutic agent. In some embodiments, the therapeutic effect of the one therapeutic agent completely overlaps with the therapeutic effect of the another therapeutic agent. In some embodiments, “temporal proximity” means that administration of one therapeutic agent occurs within a time period before or after the administration of another therapeutic agent, such that there is a synergistic effect between the one therapeutic agent and the another therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

As used herein a “kit” refers to a combination of components, such as a combination of the compositions herein and another item for a purpose including, but not limited to, reconstitution, activation and instruments/devices for delivery, administration, diagnosis and assessment of a biological activity or property. Kits optionally include instructions of use.

The present disclosure is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The present disclosure is intended to include all isomers of the compound, which refers to and includes, optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers.

### HER2 Antibodies

The HER2 antibodies suitable for the combinations or methods of the disclosure specifically bind the human HER2 in soluble form, or membrane bound (i.e., when expressed on a cell surface). The disclosure further provides monoclonal antibodies that specifically bind HER2. HER2. These antibodies are collectively referred to herein as “HER2” antibodies.

The HER2 antibodies suitable for the combinations or methods disclosed herein bind to a HER2 epitope with an equilibrium dissociation constant (K_{d} or K_{D}) of ≤1 μM, e.g., ≤100 nM, preferably ≤10 nM, and more preferably ≤1 nM. For example, the HER2 antibodies provided herein exhibit a K_{d} in the range approximately between ≤1 nM to about 1 pM.

The HER2 antibodies disclosed herein serve to modulate, block, inhibit, reduce, antagonize, neutralize or otherwise interfere with the functional activity of HER2. HER2. Functional activities of HER2 include for example, modulation of PI3K-Akt pathway activity. For example, the HER2 antibodies completely or partially inhibit HER2 functional activity by partially or completely modulating, blocking, inhibiting, reducing antagonizing, neutralizing, or otherwise interfering with PI3K-Akt pathway activity. PI3K-Akt pathway activity is assessed using any art-recognized method for detecting PI3K-Akt pathway activity, including, but not limited to detecting levels of phosphorylated Akt in the presence and absence of an antibody or antigen binding fragment disclosed herein.

The HER2 antibodies are considered to completely modulate, block, inhibit, reduce, antagonize, neutralize or otherwise interfere with HER2 functional activity when the level of HER2 functional activity in the presence of the HER2 antibody is decreased by at least 95%, e.g., by 96%, 97%, 98%, 99% or 100% as compared to the level of HER2 functional activity in the absence of binding with a HER2 antibody described herein. The HER2 antibodies are considered to partially modulate, block, inhibit, reduce, antagonize, neutralize or otherwise interfere with HER2 functional activity when the level of HER2 activity in the presence of the HER2 antibody is decreased by less than 95%, e.g., 10%, 20%, 25%, 30%, 40%, 50%, 60%, 75%, 80%, 85% or 90% as compared to the level of HER2 activity in the absence of binding with a HER2 antibody described herein.

Exemplary antibodies disclosed herein include, for example, the XMT 1517 antibody, the XMT 1518 antibody, the XMT 1519 antibody, and the XMT 1520 antibody. These antibodies show specificity for human HER2 and they have been shown to inhibit the functional activity of HER2 in vitro.

Each of the HER2 monoclonal antibodies described herein includes a heavy chain (HC), heavy chain variable region (VH), light chain (LC), and a light chain variable region (VL), as shown in the amino acid and corresponding nucleic acid sequences presented below. The variable heavy chain region and variable light chain region for each antibody are shaded in the amino acid sequences below. The complementarity determining regions (CDRs) of the heavy chain and the light chain are underlined in the amino acid sequences presented below. The amino acids encompassing the complementarity determining regions (CDR) are as defined by E. A. Kabat et al. (See Kabat, E. A., et al., Sequences of Protein of immunological interest, Fifth Edition, US Department of Health and Human Services, US Government Printing Office (1991)).

>XMT 1517 Heavy Chain Amino Acid Sequence (Heavy
chain variable region (SEQ ID NO: 9) + IgG1 Heavy
chain constant region (SEQ ID NO: 32))
(SEQ ID NO: 1)
[QVQLVESGGGVVQPGRSLRLSCAASGFTFSSYGMHWVRQAPGKGLEWVA
VIWYDGSNKYYADSVKGRFTISRDNSKNTLYLQMNSLRAEDTAVYYCAKE
APYYAKDYMDVWGKGTTVTVSS]ASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTI
PPVLDSDGSFFLYSKLTVDKSRWQQGNVFSCSVMHEALHNHYTQKSLSLS
PG*
(SEQ ID NO: 17)
CDRH1: FTFSSYGMH
(SEQ ID NO: 18)
CDRH2: VIWYDGSNKYYADSVKG
(SEQ ID NO: 19)
CDRH3: EAPYYAKDYMDV
>XMT 1517 Heavy Chain variable region nucleic
acid sequence
(SEQ ID NO: 34)
CAGGTGCAGCTGGTGGAGTCTGGGGGAGGCGTGGTCCAGCCTGGGAGGTC
CCTGAGACTCTCCTGTGCAGCGTCTGGATTCACCTTCAGTAGCTATGGCA
TGCACTGGGTCCGCCAGGCTCCAGGCAAGGGGCTGGAGTGGGIGGCAGTT
ATATGGTATGATGGAAGTAATAAATACTATGCAGACTCCGTGAAGGGCCG
ATTCACCATCTCCAGAGACAATTCCAAGAACACGCTGTATCTGCAAATGA
ACAGCCTGAGAGCCGAGGACACGGCGGIGTACTACTGCGCCAAGGAAGCT
CCCTACTACGCTAAAGATTACATGGACGTATGGGGCAAGGGTACAACTGT
CACCGTCTCCTCA
>XMT 1517 Light Chain Amino Acid Sequence (Light
chain variable region (SEQ ID NO: 10) + Light
chain constant region (SEQ ID NO: 33))
(SEQ ID NO: 2)
[EIVLTQSPGTLSLSPGERATLSCRASQSVSSDYLAWYQQKPGQAPRLLI
YGASSRATGIPDRFSGSGSGTDFTLTISRLEPEDFAVYYCQQYVSYWTFG
GGTKVEIK]RTVAAPSVFIFPPSDEQLKSGTASVVCLLNNFYPREAKVQW
KVDNALQSGNSQESVTEQDSKDSTYSLSSTLTLSKADYEKHKVYACEV
THQGLSSPVTKSFNRGEC
(SEQ ID NO: 20)
CDRL1: RASQSVSSDYLA
(SEQ ID NO: 21)
CDRL2: GASSRAT
(SEQ ID NO: 22)
CDRL3: QQYVSYWT
>XMT 1517 Light Chain variable region nucleic
acid sequence
(SEQ ID NO: 35)
GAAATTGTGTTGACGCAGTCTCCAGGCACCCTGTCTTTGTCTCCAGGGGA
AAGAGCCACCCTCTCCTGCAGGGCCAGTCAGAGTGTTAGCAGCGACTACT
TAGCCTGGTACCAGCAGAAACCTGGCCAGGCTCCCAGGCTCCTCATCTAT
GGTGCATCCAGCAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTACGTCAGTTACTGGACTTTTGGCGGA
GGGACCAAGGTTGAGATCAAA
>XMT 1518 Heavy Chain Amino Acid Sequence (Heavy
chain variable region (SEQ ID NO: 11) + IgG1 Heavy
chain constant (SEQ ID NO: 32))
(SEQ ID NO: 3)
[QVQLVESGGGVVQPGRSLRLSCAASGFTFSSYGMHWVRQAPGKGLEWVA
GIWWDGSNEKYADSVKGRFTISRDNSKNTLYLQMNSLRAEDTAVYYCAKE
APYYAKDYMDVWGKGTTVTVSS]ASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PG*
(SEQ ID NO: 17)
CDRH1: FTFSSYGMH
(SEQ ID NO: 23)
CDRH2: GIWWDGSNEKYADSVKG
(SEQ ID NO: 19)
CDRH3: EAPYYAKDYMDV
>XMT 1518 Light Chain Amino Acid Sequence (Light
chain variable region (SEQ ID NO: 12) + Light
chain constant (SEQ ID NO: 33))
(SEQ ID NO: 4)
[EIVLTQSPGTLSLSPGERATLSCRASQSVSSDYLAWYQQKPGQAPRLLI
YGASRRATGIPDRFSGSGSGTDFTLTISRLEPEDFAVYYCQQYVSYWTFG
GGTKVEIK]RTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC
(SEQ ID NO: 20)
CDRL1: RASQSVSSDYLA
(SEQ ID NO: 24)
CDRL2: GASRRAT
(SEQ ID NO: 22)
CDRL3: QQYVSYWT
>XMT 1519 Heavy Chain Amino Acid Sequence (Heavy
chain variable region (SEQ ID NO: 13) + IgG1
Heavy chain constant region (SEQ ID NO: 32))
(SEQ ID NO: 5)
[EVQLVESGGGLVQPGGSLRLSCAASGFTFSSYSMNWVRQAPGKGLEWVS
YISSSSSTIYYADSVKGRFTISRDNAKNSLYLQMNSLRAEDTAVYYCARG
GHGYFDLWGRGTLVTVSS]ASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG*
(SEQ ID NO: 25)
CDRH1: FTFSSYSMN
(SEQ ID NO: 26)
CDRH2: YISSSSSTIYYADSVKG
(SEQ ID NO: 27)
CDRH3: GGHGYFDL
>XMT 1519 Heavy Chain variable region nucleic acid
sequence
(SEQ ID NO: 36)
GAGGTGCAGCTGGTGGAGTCTGGGGGAGGCTTGGTACAGCCTGGGGGGTC
CCTGAGACTCTCCTGTGCAGCCTCTGGATTCACCTTCAGTAGCTATAGCA
TGAACTGGGTCCGCCAGGCTCCAGGGAAGGGGCTGGAGTGGGTTTCATAC
ATTAGTAGTAGTAGTAGTACCATATACTACGCAGACTCTGTGAAGGGCCG
ATICACCATCTCCAGAGACAATGCCAAGAACTCACTGTATCTGCAAATGA
ACAGCCTGAGAGCTGAGGACACGGCGGTGTACTACTGCGCCAGAGGTGGA
CACGGATATTTCGACCTATGGGGGAGAGGTACCTTGGTCACCGTCTCCTC
A
>XMT 1519 Light Chain Amino Acid Sequence (Light
chain variable region (SEQ ID NO: 14) + Light
chain constant region (SEQ ID NO: 33))
(SEQ ID NO: 6)
[EIVLTQSPGTLSLSPGERATLSCRASQSVSSSYLAWYQQKPGQAPRLLI
YGASSRATGIPDRFSGSGSGTDFTLTISRLEPEDFAVYYCQQYHHSPLTF
GGGTKVEIK]RTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC
(SEQ ID NO: 28)
CDRL1: RASQSVSSSYLA
(SEQ ID NO: 21)
CDRL2: GASSRAT
(SEQ ID NO: 29)
CDRL3: QQYHHSPLT
>XMT 1519 Light Chain variable region nucleic acid
sequence
(SEQ ID NO: 37)
GAAATTGTGTTGACGCAGTCTCCAGGCACCCTGTCTTTGTTCTCCAGGGG
AAAGAGCCACCCTCTCCTGCAGGGCCAGTCAGAGTGTTAGCAGCAGCTAC
TTAGCCTGGTACCAGCAGAAACCTGGCCAGGCTCCCAGGCTCCTCATCTA
TGGTGCATCCAGCAGGGCCACTGGCATCCCAGACAGGTTCAGTGGCAGTG
GGTCTGGGACAGACTTCACTCTCACCATCAGCAGACTGGAGCCTGAAGAT
TTTGCAGTGTATTACTGTCAGCAGTACCACCACAGTCCTCTCACTTTTGG
CGGAGGGACCAAGGTTGAGATCAAA
>XMT 1520 Heavy Chain Amino Acid Sequence (Heavy
chain variable region (SEQ ID NO: 15) + IgG1 Heavy
chain constant region (SEQ ID NO: 32))
(SEQ ID NO: 7)
[EVQLVESGGGLVQPGGSLRLSCAASGFTFSGRSMNWVRQAPGKGLEWVS
YISSDSRTIYYADSVKGRFTISRDNAKNSLYLQMNSLRAEDTAVYYCARG
GHGYFDLWGRGTLVTVSS]ASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG*
(SEQ ID NO: 30)
CDRH1: FTFSGRSMN
(SEQ ID NO: 31)
CDRH2: YISSDSRTIYYADSVKG
(SEQ ID NO: 27)
CDRH3: GGHGYFDL
>XMT 1520 Light Chain Amino Acid Sequence (Light
chain variable region (SEQ ID NO: 16) + Light
chain constant region (SEQ ID NO: 33))
(SEQ ID NO: 8)
[EIVLTQSPGTLSLSPGERATLSCRASQSVSSSYLAWYQQKPGQAPRLLI
YGASSRATGIPDRFSGSGSGTDFTLTISRLEPEDFAVYYCQQYHHSPLTF
GGGTKVEIK]RTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC
(SEQ ID NO: 28)
CDRL1: RASQSVSSSYLA
(SEQ ID NO: 21)
CDRL2: GASSRAT
(SEQ ID NO: 29)
CDRL3: QQYHHSPLT

Also included in the disclosure are antibodies and antigen binding fragments thereof that bind to the same epitope or cross compete for binding to the same epitope as the antibodies and antigen binding fragments thereof described herein. For example, antibodies and antigen binding fragments disclosed herein specifically bind to HER2, wherein the antibody or fragment binds to an epitope that includes one or more amino acid residues on human HER2 (e.g., GenBank Accession No. P04626.1).

Antibodies and antigen binding fragments thereof disclosed herein specifically bind to an epitope on the full-length human HER2 receptor comprising the amino acid sequence:

(SEQ ID NO: 38)

| | |
|---|---|
| 1 | MELAALCRWG LLLALLPPGA ASTQVCTGTD MKLRLPASPE |
| | THLDMLRHLY |
| 51 | QGCQVVQGNL ELTYLPTNAS LSFLQDIQEV QGYVLIAHNQ |
| | VRQVPLQRLR |
| 101 | IVRGTQLFED NYALAVLDNG DPLNNTTPVT GASPGGLREL |
| | QLRSLTEILK |
| 151 | GGVLIQRNPQ LCYQDTILWK DIFHKNNQLA LTLIDTNRSR |
| | ACHPCSPMCK |
| 201 | GSRCWGESSE DCQSLTRTVC AGGCARCKGP LPTDCCHEQC |
| | AAGCTGPKHS |
| 251 | DCLACLHFNH SGICELHCPA LVTYNTDTFE SMPNPEGRYT |
| | FGASCVTACP |
| 301 | YNYLSTDVGS CTLVCPLHNQ EVTAEDGTQR CEKCSKPCAR |
| | VCYGLGMEHL |
| 351 | REVRAVTSAN IQEFAGCKKI FGSLAFLPES FDGDPASNTA |
| | PLQPEQLQVF |
| 401 | ETLEEITGYL YISAWPDSLP DLSVFQNLQV IRGRILHNGA |
| | YSLTLQGLGI |
| 451 | SWLGLRSLRE LGSGLALIHH NTHLCFVHTV PWDQLFRNPH |
| | QALLHTANRP |
| 501 | EDECVGEGLA CHQLCARGHC WGPGPTQCVN CSQFLRGQEC |
| | VEECRVLQGL |
| 551 | PREYVNARHC LPCHPECQPQ NGSVTCFGPE ADQCVACAHY |
| | KDPPFCVARC |
| 601 | PSGVKPDLSY MPIWKFPDEE GACQPCPINC THSCVDLDDK |
| | GCPAEQRASP |
| 651 | LTSIISAVVG ILLVVVLGVV FGILIKRRQQ KIRKYTMRRL |
| | LQETELVEPL |
| 701 | TPSGAMPNQA QMRILKETEL RKVKVLGSGA FGTVYKGIWI |
| | PDGENVKIPV |
| 751 | AIKVLRENTS PKANKEILDE AYVMAGVGSP YVSRLLGICL |
| | TSTVQLVTQL |
| 801 | MPYGCLLDHV RENRGRLGSQ DLLNWCMQIA KGMSYLEDVR |
| | LVHRDLAARN |
| 851 | VLVKSPNHVK ITDFGLARLL DIDETEYHAD GGKVPIKWMA |
| | LESILRRRFT |
| 901 | HQSDVWSYGV TVWELMTFGA KPYDGIPARE IPDLLEKGER |
| | LPQPPICTID |
| 951 | VYMIMVKCWM IDSECRPRFR ELVSEFSRMA RDPQRFVVIQ |
| | NEDLGPASPL |
| 1001 | DSTFYRSLLE DDDMGDLVDA EEYLVPQQGF FCPDPAPGAG |
| | GMVHHRHRSS |
| 1051 | STRSGGGDLT LGLEPSEEEA PRSPLAPSEG AGSDVFDGDL |
| | GMGAAKGLQS |
| 1101 | LPTHDPSPLQ RYSEDPTVPL PSETDGYVAP LTCSPQPEYV |
| | NQPDVRPQPP |
| 1151 | SPREGPLPAA RPAGATLERP KTLSPGKNGV VKDVFAFGGA |
| | VENPEYLTPQ |
| 1201 | GGAAPQPHPP PAFSPAFDNL YYWDQDPPER GAPPSTFKGT |
| | PTAENPEYLG |
| 1251 | LDVPV |

Antibodies and antigen binding fragments thereof disclosed herein specifically bind to an epitope on the extracellular domain (ECD) of the human HER2 receptor comprising the amino acid sequence:

(SEQ ID NO: 39)

| | |
|---|---|
| 1 | TQVCTGTDMK LRLPASPETH LDMLRHLYQG CQVVQGNLEL |
| | TYLPTNASLS |
| 51 | FLQDIQEVQG YVLIAHNQVR QVPLQRLRIV RGTQLFEDNY |
| | ALAVLDNGDP |
| 101 | LNNTTPVTGA SPGGLRELQL RSLTEILKGG VLIQRNPQLC |
| | YQDTILWKDI |
| 151 | FHKNNQLALT LIDTNRSRAC HPCSPMCKGS RCWGESSEDC |
| | QSLTRTVCAG |
| 201 | GCARCKGPLP TDCCHEQCAA GCTGPKHSDC LACLHFNHSG |
| | ICELHCPALV |
| 251 | TYNTDTFESM PNPEGRYTFG ASCVTACPYN YLSTDVGSCT |
| | LVCPLHNQEV |
| 301 | TAEDGTQRCE KCSKPCARVC YGLGMEHLRE VRAVTSANIQ |
| | EFAGCKKIFG |
| 351 | SLAFLPESFD GDPASNTAPL QPEQLQVFET LEEITGYLYI |
| | SAWPDSLPDL |
| 401 | SVFQNLQVIR GRILHNGAYS LTLQGLGISW LGLRSLRELG |
| | SGLALIHHNT |
| 451 | HLCFVHTVPW DQLFRNPHQA LLHTANRPED ECVGEGLACH |
| | QLCARGHCWG |
| 501 | PGPTQCVNCS QFLRGQECVE ECRVLQGLPR EYVNARHCLP |
| | CHPECQPQNG |
| 551 | SVTCFGPEAD QCVACAHYKD PPFCVARCPS GVKPDLSYMP |
| | IWKFPDEEGA |
| 601 | CQPCPINCTH SCVDLDDKGC PAEQRASPLT |

The antibodies of the present disclosure exhibit HER2 binding characteristics that differ from antibodies described in the art. Particularly, the antibodies disclosed herein bind to a different epitopes of HER2, in that they cross-block each other but not trastuzumab, pertuzumab, Fab37 or chA21 from binding to HER2. Further, as opposed to the known antibodies, the antibodies disclosed herein can internalize efficiently into HER2-expressing cells without promoting cell proliferation.

The antibodies disclosed herein are fully human monoclonal antibodies that bind to novel epitopes and/or have other favorable properties for therapeutic use. Exemplary properties include, but are not limited to, favorable binding characteristics to cancer cells expressing human HER2 at high or low levels, specific binding to recombinant human and cynomolgus monkey HER2, efficient internalization upon binding to HER2, high capacity for killing cancer cells expressing high or low levels of HER2 when administered as an antibody drug conjugate (ADC), no substantial agonistic effect on the proliferation of HER2-expressing cancer cells, and provide for effective antibody-dependent cellular cytotoxicity (ADCC)-mediated killing of HER2-expressing cells, as well as any combination of the foregoing properties.

The antibodies disclosed herein also include an antibody or antigen binding fragment thereof that specifically binds to an epitope of the human HER2 receptor that includes residues 452 to 531 of the extracellular domain of the human HER2 receptor, for example, residues 474 to 553 of SEQ ID NO: 38 or residues 452 to 531 of SEQ ID NO: 39.

The antibodies disclosed herein include an antibody or an antigen binding fragment thereof that binds at least a portion of the N-terminus of domain IV of human HER2 receptor but does not cross-compete with an antibody that binds to epitope 4D5 of the human HER2 receptor. For example, the antibodies or antigen binding fragments thereof described herein do not cross-compete with trastuzumab for binding to the human HER2 receptor, as trastuzumab is known to bind epitope 4D5 of the human HER2 receptor. As used herein, the term epitope 4D5 of the human HER2 receptor refers to amino acid residues 529 to 627 of the extracellular domain of the human HER2 receptor, for example residues 551 to 649 of SEQ ID NO: 38 or residues 529 to 627 of SEQ ID NO: 39. In some embodiments, the antibody or antigen binding fragment thereof also binds at least one epitope on cynomolgus monkey HER2 receptor.

The antibodies disclosed herein also include an antibody or antigen binding fragment thereof that specifically binds to an epitope of the human HER2 receptor that includes residues 452 to 500 of the extracellular domain of the human HER2 receptor, for example, residues 474 to 522 of SEQ ID NO: 38 or residues 452 to 500 of SEQ ID NO: 39.

The antibodies disclosed herein also include an antibody or antigen binding fragment thereof that specifically binds to an epitope of the human HER2 receptor that includes at least one of amino acid residue selected from the group consisting of amino acid residues E521, L525 and R₅₃₀ of the extracellular domain of the human HER2 receptor, e.g., residues 543, 547, and 552 of SEQ ID NO: 38, and residues 521, 525, and 530 of SEQ ID NO: 39. For example, the antibodies disclosed herein include an antibody or antigen binding fragment thereof that specifically binds to an epitope of the extracellular domain of the human HER2 receptor that includes at least two amino acid residues selected from the group consisting of amino acid residues E521, L525 and R530 of the extracellular domain of the human HER2 receptor. The antibodies disclosed herein also include an antibody or antigen binding fragment thereof that specifically binds to an epitope of the human HER2 receptor that includes at least amino acid residues E521, L525 and R530 of the extracellular domain of the human HER2 receptor. In some embodiments, any or all of these antibodies or antigen binding fragments thereof also bind at least one epitope on cynomolgus monkey HER2 receptor.

The antibodies disclosed herein also include an antibody or an antigen binding fragment thereof that binds to at least a portion of domain III and at least a portion of the N-terminus of domain IV of human HER2 receptor but does not cross-compete with Fab37 monoclonal antibody or an antibody that binds to epitope 4D5 of the human HER2 receptor. For example, the antibodies or antigen binding fragments thereof described herein do not cross-compete with the Fab37 monoclonal antibody and/or trastuzumab for binding to the human HER2 receptor. In some embodiments, the antibody or antigen binding fragment thereof also binds at least one epitope on cynomolgus monkey HER2 receptor.

The antibodies disclosed herein also include an antibody or antigen binding fragment thereof that specifically binds to an epitope of the human HER2 receptor that includes residues 520 to 531 of the extracellular domain of the human HER2 receptor, for example, residues 542 to 553 of SEQ ID NO: 38 or residues 520 to 531 of SEQ ID NO: 39.

The antibodies disclosed herein also include an antibody or antigen binding fragment thereof that specifically binds to an epitope of the human HER2 receptor that includes at least one amino acid residue selected from the group consisting of residues C453, H456, H473, N476, R495, G496, H497 and W499 of the extracellular domain of the human HER2 receptor, e.g., residues 475, 478, 495, 498, 517, 518, 519, and 521 of SEQ ID NO: 38 or residues 453, 456, 473, 476, 495, 496, 497 and 499 of SEQ ID NO: 39. For example, the antibodies disclosed herein include an antibody or antigen binding fragment thereof that specifically binds to an epitope of the extracellular domain of the human HER2 receptor that includes at least two amino acid residues selected from the group consisting of amino acid residues C453, H456, H473, N476, R495, G496, H497 and W499 of the extracellular domain of the human HER2 receptor. For example, the antibodies disclosed herein include an antibody or antigen binding fragment thereof that specifically binds to an epitope of the extracellular domain of the human HER2 receptor that includes at least three amino acid residues selected from the group consisting of amino acid residues C453, H456, H473, N476, R495, G496, H497 and W499 of the extracellular domain of the human HER2 receptor. For example, the antibodies disclosed herein include an antibody or antigen binding fragment thereof that specifically binds to an epitope of the extracellular domain of the human HER2 receptor that includes at least four amino acid residues selected from the group consisting of amino acid residues C453, H456, H473, N476, R495, G496, H497 and W499 of the extracellular domain of the human HER2 receptor. For example, the antibodies disclosed herein include an antibody or antigen binding fragment thereof that specifically binds to an epitope of the extracellular domain of the human HER2 receptor that includes at least five amino acid residues selected from the group consisting of amino acid residues C453, H456, H473, N476, R495, G496, H497 and W499 of the extracellular domain of the human HER2 receptor. For example, the antibodies disclosed herein include an antibody or antigen binding fragment thereof that specifically binds to an epitope of the extracellular domain of the human HER2 receptor that includes at least six amino acid residues selected from the group consisting of amino acid residues C453, H456, H473, N476, R495, G496, H497 and W499 of the extracellular domain of the human HER2 receptor. For example, the antibodies disclosed herein include an antibody or antigen binding fragment thereof that specifically binds to an epitope of the extracellular domain of the human HER2 receptor that includes at least amino acid residues C453, H456, H473, N476, R495, G496, H497 and W499 of the extracellular domain of the human HER2 receptor. In some embodiments, any or all of these antibodies or antigen binding fragments thereof also bind at least one epitope on cynomolgus monkey HER2 receptor.

The antibodies disclosed herein also include an antibody or antigen binding fragment thereof that specifically binds to an epitope of the human HER2 receptor that includes at least one amino acid residue selected from the group consisting of residues C453, H473, N476, R495, H497 and W499 of the extracellular domain of the human HER2 receptor, e.g., residues 475, 495, 498, 517, 519, and 521 of SEQ ID NO: 38 or residues 453, 473, 476, 495, 497 and 499 of SEQ ID NO: 39. For example, the antibodies disclosed herein include an antibody or antigen binding fragment thereof that specifically binds to an epitope of the extracellular domain of the human HER2 receptor that includes at least two amino acid residues selected from the group consisting of amino acid residues C453, H473, N476, R495, H497 and W499 of the extracellular domain of the human HER2 receptor. For example, the antibodies disclosed herein include an antibody or antigen binding fragment thereof that specifically binds to an epitope of the extracellular domain of the human HER2 receptor that includes at least three amino acid residues selected from the group consisting of amino acid residues C453, H473, N476, R495, H497 and W499 of the extracellular domain of the human HER2 receptor. For example, the antibodies disclosed herein include an antibody or antigen binding fragment thereof that specifically binds to an epitope of the extracellular domain of the human HER2 receptor that includes at least four amino acid residues selected from the group consisting of amino acid residues C453, H473, N476, R495, H497 and W499 of the extracellular domain of the human HER2 receptor. For example, the antibodies disclosed herein include an antibody or antigen binding fragment thereof that specifically binds to an epitope of the extracellular domain of the human HER2 receptor that includes at least five amino acid residues selected from the group consisting of amino acid residues C453, H473, N476, R495, H497 and W499 of the extracellular domain of the human HER2 receptor. For example, the antibodies disclosed herein include an antibody or antigen binding fragment thereof that specifically binds to an epitope of the extracellular domain of the human HER2 receptor that includes at least six amino acid residues selected from the group consisting of amino acid residues C453, H473, N476, R495, H497 and W499 of the extracellular domain of the human HER2 receptor. For example, the antibodies disclosed herein include an antibody or antigen binding fragment thereof that specifically binds to an epitope of the extracellular domain of the human HER2 receptor that includes at least amino acid residues C453, H473, N476, R495, H497 and W499 of the extracellular domain of the human HER2 receptor. In some embodiments, any or all of these antibodies or antigen binding fragments thereof also bind at least one epitope on cynomolgus monkey HER2 receptor.

Exemplary monoclonal antibodies disclosed herein include, for example, the XMT 1517 antibody, the XMT 1518 antibody, the XMT 1519 antibody and the XMT 1520 antibody described herein. Alternatively, the monoclonal antibody is an antibody that cross block each other but do not bind to the same epitope as trastuzumab, pertuzumab, Fab37 or chA21 (that bind to specific epitopes on domain IV, domain II, domain III and domain I of HER2 respectively) or biosimilars thereof. These antibodies are respectively referred to herein as “HER2” antibodies. HER2 antibodies include fully human monoclonal antibodies, as well as humanized monoclonal antibodies and chimeric antibodies. These antibodies show specificity for human HER2, and they have been shown to modulate, e.g., block, inhibit, reduce, antagonize, neutralize or otherwise interfere with the PI3K-Akt pathway which promotes cell survival by reducing levels of phosphorylated AKT. These antibodies internalize from the cell surface of HER2-expressing cells at a rate that is the same or substantially similar to the rate at which trastuzumab or a biosimilar thereof internalizes. For example, these antibodies and antigen binding fragments have a rate of internalization that is about 50% of the total surface bound at time 0 being internalized by 4 hours.

The antibodies disclosed herein contain a heavy chain having an amino acid sequence at least 90%, 91%, 92%, 93%, 94%, 95%, 96%, 97% 98%, 99% or more identical to a sequence selected from the group consisting of SEQ ID NOs: 1, 3, 5, and 7 and a light chain having an amino acid sequence at least 90%, 91%, 92%, 93%, 94%, 95%, 96%, 97% 98%, 99% or more identical to a sequence selected from the group consisting of SEQ ID NOs: 2, 4, 6, and 8.

The antibodies disclosed herein contain a combination of heavy chain and light chain amino acid sequences selected from the group consisting of (i) a heavy chain amino acid sequence at least 90%, 91%, 92%, 93%, 94%, 95%, 96%, 97% 98%, 99% or more identical to the amino acid sequence of SEQ ID NO: 1 and a light chain amino acid sequence at least 90%, 91%, 92%, 93%, 94%, 95%, 96%, 97% 98%, 99% or more identical to the amino acid sequence of SEQ ID NO: 2; (ii) a heavy chain amino acid sequence at least 90%, 91%, 92%, 93%, 94%, 95%, 96%, 97% 98%, 99% or more identical to the amino acid sequence of SEQ ID NO: 3 and a light chain amino acid sequence at least 90%, 91%, 92%, 93%, 94%, 95%, 96%, 97% 98%, 99% or more identical to the amino acid sequence of SEQ ID NO: 4; (iii) a heavy chain amino acid sequence at least 90%, 91%, 92%, 93%, 94%, 95%, 96%, 97% 98%, 99% or more identical to the amino acid sequence of SEQ ID NO: 5 and a light chain amino acid sequence at least 90%, 91%, 92%, 93%, 94%, 95%, 96%, 97% 98%, 99% or more identical to the amino acid sequence of SEQ ID NO: 6; and (iv) a heavy chain amino acid sequence at least 90%, 91%, 92%, 93%, 94%, 95%, 96%, 97% 98%, 99% or more identical to the amino acid sequence of SEQ ID NO: 7 and a light chain amino acid sequence at least 90%, 91%, 92%, 93%, 94%, 95%, 96%, 97% 98%, 99% or more identical to the amino acid sequence of SEQ ID NO: 8.

In some embodiments, the antibodies disclosed herein contain a heavy chain amino acid sequence at least 90%, 91%, 92%, 93%, 94%, 95%, 96%, 97% 98%, 99% or more identical to the amino acid sequence of SEQ ID NO: 1 and a light chain amino acid sequence at least 90%, 91%, 92%, 93%, 94%, 95%, 96%, 97% 98%, 99% or more identical to the amino acid sequence of SEQ ID NO: 2.

In some embodiments, the antibodies disclosed herein contain a heavy chain amino acid sequence at least 90%, 91%, 92%, 93%, 94%, 95%, 96%, 97% 98%, 99% or more identical to the amino acid sequence of SEQ ID NO: 3 and a light chain amino acid sequence at least 90%, 91%, 92%, 93%, 94%, 95%, 96%, 97% 98%, 99% or more identical to the amino acid sequence of SEQ ID NO: 4.

In some embodiments, the antibodies disclosed herein contain a heavy chain amino acid sequence at least 90%, 91%, 92%, 93%, 94%, 95%, 96%, 97% 98%, 99% or more identical to the amino acid sequence of SEQ ID NO: 5 and a light chain amino acid sequence at least 90%, 91%, 92%, 93%, 94%, 95%, 96%, 97% 98%, 99% or more identical to the amino acid sequence of SEQ ID NO: 6.

In some embodiments, the antibodies disclosed herein contain a heavy chain amino acid sequence at least 90%, 91%, 92%, 93%, 94%, 95%, 96%, 97% 98%, 99% or more identical to the amino acid sequence of SEQ ID NO: 7 and a light chain amino acid sequence at least 90%, 91%, 92%, 93%, 94%, 95%, 96%, 97% 98%, 99% or more identical to the amino acid sequence of SEQ ID NO: 8.

The antibodies disclosed herein contain a heavy chain having an amino acid sequence selected from the group consisting of SEQ ID NOs: 1, 3, 5, and 7 and a light chain having an amino acid sequence selected from the group consisting of SEQ ID NOs: 2, 4, 6, and 8 respectively.

The antibodies disclosed herein contain a combination of heavy chain and light chain amino acid sequences selected from the group consisting of (i) the heavy chain amino acid sequence of SEQ ID NO: 1 and the light chain amino acid sequence of SEQ ID NO: 2; (ii) the heavy chain amino acid sequence of SEQ ID NO: 3 and the light chain amino acid sequence of SEQ ID NO: 4; (iii) the heavy chain amino acid sequence of SEQ ID NO: 5 and the light chain amino acid sequence of SEQ ID NO: 6; and (iv) the heavy chain amino acid sequence of SEQ ID NO: 7 and the light chain amino acid sequence of SEQ ID NO: 8.

In some embodiments, the antibodies disclosed herein contain the heavy chain amino acid sequence of SEQ ID NO: 1 and the light chain amino acid sequence of SEQ ID NO: 2.

In some embodiments, the antibodies disclosed herein contain the heavy chain amino acid sequence of SEQ ID NO: 3 and the light chain amino acid sequence of SEQ ID NO: 4.

In some embodiments, the antibodies disclosed herein contain the heavy chain amino acid sequence of SEQ ID NO: 5 and the light chain amino acid sequence of SEQ ID NO: 6.

In some embodiments, the antibodies disclosed herein contain the heavy chain amino acid sequence of SEQ ID NO: 7 and the light chain amino acid sequence of SEQ ID NO: 8.

The antibodies disclosed herein contain a heavy chain variable region having an amino acid sequence at least 90%, 91%, 92%, 93%, 94%, 95%, 96%, 97% 98%, 99% or more identical to a sequence selected from the group consisting of SEQ ID NOs: 9, 11, 13, and 15 and a light chain variable region having an amino acid sequence at least 90%, 91%, 92%, 93%, 94%, 95%, 96%, 97% 98%, 99% or more identical to a sequence selected from the group consisting of SEQ ID NOs: 10, 12, 14, and 16.

The antibodies disclosed herein contain a combination of heavy chain variable region and light chain variable region amino acid sequences selected from the group consisting of (i) a heavy chain variable region amino acid sequence at least 90%, 91%, 92%, 93%, 94%, 95%, 96%, 97% 98%, 99% or more identical to the amino acid sequence of SEQ ID NO: 9 and a light chain variable region amino acid sequence at least 90%, 91%, 92%, 93%, 94%, 95%, 96%, 97% 98%, 99% or more identical to the amino acid sequence of SEQ ID NO: 10; (ii) a heavy chain variable region amino acid sequence at least 90%, 91%, 92%, 93%, 94%, 95%, 96%, 97% 98%, 99% or more identical to the amino acid sequence of SEQ ID NO: 11 and a light chain variable region amino acid sequence at least 90%, 91%, 92%, 93%, 94%, 95%, 96%, 97% 98%, 99% or more identical to the amino acid sequence of SEQ ID NO: 12; (iii) a heavy chain variable region amino acid sequence at least 90%, 91%, 92%, 93%, 94%, 95%, 96%, 97% 98%, 99% or more identical to the amino acid sequence of SEQ ID NO: 13 and a light chain variable region amino acid sequence at least 90%, 91%, 92%, 93%, 94%, 95%, 96%, 97% 98%, 99% or more identical to the amino acid sequence of SEQ ID NO: 14; and (iv) a heavy chain variable region amino acid sequence at least 90%, 91%, 92%, 93%, 94%, 95%, 96%, 97% 98%, 99% or more identical to the amino acid sequence of SEQ ID NO: 15 and a light variable region chain amino acid sequence at least 90%, 91%, 92%, 93%, 94%, 95%, 96%, 97% 98%, 99% or more identical to the amino acid sequence of SEQ ID NO: 16.

In some embodiments, the antibodies disclosed herein contain a heavy chain variable region amino acid sequence at least 90%, 91%, 92%, 93%, 94%, 95%, 96%, 97% 98%, 99% or more identical to the amino acid sequence of SEQ ID NO: 9 and a light chain variable region amino acid sequence at least 90%, 91%, 92%, 93%, 94%, 95%, 96%, 97% 98%, 99% or more identical to the amino acid sequence of SEQ ID NO: 10.

In some embodiments, the antibodies disclosed herein contain a heavy chain variable region amino acid sequence at least 90%, 91%, 92%, 93%, 94%, 95%, 96%, 97% 98%, 99% or more identical to the amino acid sequence of SEQ ID NO: 11 and a light chain variable region amino acid sequence at least 90%, 91%, 92%, 93%, 94%, 95%, 96%, 97% 98%, 99% or more identical to the amino acid sequence of SEQ ID NO: 12.

In some embodiments, the antibodies disclosed herein contain a heavy chain variable region amino acid sequence at least 90%, 91%, 92%, 93%, 94%, 95%, 96%, 97% 98%, 99% or more identical to the amino acid sequence of SEQ ID NO: 13 and a light chain variable region amino acid sequence at least 90%, 91%, 92%, 93%, 94%, 95%, 96%, 97% 98%, 99% or more identical to the amino acid sequence of SEQ ID NO: 14.

In some embodiments, the antibodies disclosed herein contain a heavy chain variable region amino acid sequence at least 90%, 91%, 92%, 93%, 94%, 95%, 96%, 97% 98%, 99% or more identical to the amino acid sequence of SEQ ID NO: 15 and a light variable region chain amino acid sequence at least 90%, 91%, 92%, 93%, 94%, 95%, 96%, 97% 98%, 99% or more identical to the amino acid sequence of SEQ ID NO: 16.

The antibodies disclosed herein contain a heavy chain variable region an amino acid sequence selected from the group consisting of SEQ ID NOs: 9, 11, 13, and 15 and a light chain variable region having an amino acid sequence selected from the group consisting of SEQ ID NOs: 10, 12, 14, and 16.

The antibodies disclosed herein contain a combination of heavy chain variable region and light chain variable region amino acid sequences selected from the group consisting of (i) the heavy chain variable region amino acid sequence of SEQ ID NO: 9 and the light chain variable region amino acid sequence of SEQ ID NO: 10; (ii) the heavy chain variable region amino acid sequence of SEQ ID NO: 11 and the light chain variable region amino acid sequence of SEQ ID NO: 12; (iii) the heavy chain variable region amino acid sequence of SEQ ID NO: 13 and the light chain variable region amino acid sequence of SEQ ID NO: 14; and (iv) the heavy chain variable region amino acid sequence of SEQ ID NO: 15 and the light chain variable region amino acid sequence of SEQ ID NO: 16.

In some embodiments, the antibodies disclosed herein contain the heavy chain variable region amino acid sequence of SEQ ID NO: 9 and the light chain variable region amino acid sequence of SEQ ID NO: 10.

In some embodiments, the antibodies disclosed herein contain the heavy chain variable region amino acid sequence of SEQ ID NO: 11 and the light chain variable region amino acid sequence of SEQ ID NO: 12.

In some embodiments, the antibody disclosed herein contain a heavy chain variable region an amino acid sequence of SEQ ID NO: 13, and a light chain variable region having an amino acid sequence of SEQ ID NO: 14.

In some embodiments, the antibodies disclosed herein contain the heavy chain variable region amino acid sequence of SEQ ID NO: 15 and the light chain variable region amino acid sequence of SEQ ID NO: 16.

The three heavy chain CDRs of the antibodies disclosed herein include a heavy chain complementarity determining region 1 (CDRH1) that includes an amino acid sequence at least 90%, 91%, 92%, 93%, 94%, 95%, 96%, 97% 98%, 99% or more identical to a sequence selected from the group consisting of SEQ ID NOs: 17, 25, and 30; a heavy chain complementarity determining region 2 (CDRH2) that includes an amino acid sequence at least 90%, 91%, 92%, 93%, 94%, 95%, 96%, 97% 98%, 99% or more identical to a sequence selected from the group consisting of SEQ ID NOs: 18, 23, 26, and 31; and a heavy chain complementarity determining region 3 (CDRH3) that includes an amino acid sequence at least 90%, 91%, 92%, 93%, 94%, 95%, 96%, 97% 98%, 99% or more identical to a sequence selected from the group consisting of SEQ ID NOs: 19 and 27.

The three light chain CDRs of the antibodies disclosed herein include a light chain complementarity determining region 1 (CDRL1) that includes an amino acid sequence at least 90%, 91%, 92%, 93%, 94%, 95%, 96%, 97% 98%, 99% or more identical to a sequence selected from the group consisting of SEQ ID NOs: 20 and 28; a light chain complementarity determining region 2 (CDRL2) that includes an amino acid sequence at least 90%, 91%, 92%, 93%, 94%, 95%, 96%, 97% 98%, 99% or more identical to a sequence selected from the group consisting of SEQ ID NOs: 21 and 24; and a light chain complementarity determining region 3 (CDRL3) that includes an amino acid sequence at least 90%, 91%, 92%, 93%, 94%, 95%, 96%, 97% 98%, 99% or more identical to a sequence selected from the group consisting of SEQ ID NOs: 22 and 29.

The antibodies include a combination of heavy chain CDR and light chain CDR sequences that include a CDRH1 that includes an amino acid sequence at least 90%, 91%, 92%, 93%, 94%, 95%, 96%, 97% 98%, 99% or more identical to a sequence selected from the group consisting of SEQ ID NOs: 17, 25, and 30; a CDRH2 that includes an amino acid sequence at least 90%, 91%, 92%, 93%, 94%, 95%, 96%, 97% 98%, 99% or more identical to a sequence selected from the group consisting of SEQ ID NOs: 18, 23, 26, and 31; a CDRH3 that includes an amino acid sequence at least 90%, 91%, 92%, 93%, 94%, 95%, 96%, 97% 98%, 99% or more identical to a sequence selected from the group consisting of SEQ ID NOs: 19 and 27; a CDRL1 that includes an amino acid sequence at least 90%, 91%, 92%, 93%, 94%, 95%, 96%, 97% 98%, 99% or more identical to a sequence selected from the group consisting of SEQ ID NOs: 20 and 28; a CDRL2 that includes an amino acid sequence at least 90%, 91%, 92%, 93%, 94%, 95%, 96%, 97% 98%, 99% or more identical to a sequence selected from the group consisting of SEQ ID NOs: 21 and 24; and a CDRL3 that includes an amino acid sequence at least 90%, 91%, 92%, 93%, 94%, 95%, 96%, 97% 98%, 99% or more identical to a sequence selected from the group consisting of SEQ ID NOs: 22 and 29.

The three heavy chain CDRs of the antibodies disclosed herein include a CDRH1 that includes an amino acid sequence selected from the group consisting of SEQ ID NOs: 17, 25, and 30; a CDRH2 that includes an amino acid sequence selected from the group consisting of SEQ ID NOs: 18, 23, 26, and 31; and a CDRH3 that includes an amino acid sequence selected from the group consisting of SEQ ID NOs: 19 and 27.

The three light chain CDRs of the antibodies disclosed herein include a CDRL1 that includes an amino acid sequence selected from the group consisting of SEQ ID NOs: 20 and 28; a CDRL2 that includes an amino acid sequence selected from the group consisting of SEQ ID NOs: 21 and 24; and a CDRL3 that includes an amino acid sequence selected from the group consisting of SEQ ID NOs: 22 and 29.

The antibodies disclosed herein include a combination of heavy chain CDR and light chain CDR sequences that include a CDHR1 that includes an amino acid sequence selected from the group consisting of SEQ ID NOs: 17, 25, and 30; a CDRH2 that includes an amino acid sequence selected from the group consisting of SEQ ID NOs: 18, 23, 26, and 31; a CDRH3 that includes an amino acid sequence selected from the group consisting of SEQ ID NOs: 19 and 27; a CDRL1 that includes an amino acid sequence selected from the group consisting of SEQ ID NOs: 20 and 28; a CDRL2 that includes an amino acid sequence selected from the group consisting of SEQ ID NOs: 21 and 24; and a CDRL3 that includes an amino acid sequence selected from the group consisting of SEQ ID NOs: 22 and 29.

The antibodies disclosed herein contain a combination of heavy chain complementarity determining region and light chain complementarity determining region amino acid sequences selected from the group consisting of (i) the CDRH1 amino acid sequence of SEQ ID NO: 17, the CDRH2 amino acid sequence of SEQ ID NO: 18, the CDRH3 amino acid sequence of SEQ ID NO: 19, the CDRL1 amino acid sequence of SEQ ID NO: 20, the CDRL2 amino acid sequence of SEQ ID NO: 21, and the CDRL3 amino acid sequence of SEQ ID NO: 22; (ii) the CDRH1 amino acid sequence of SEQ ID NO: 17, the CDRH2 amino acid sequence of SEQ ID NO: 23, the CDRH3 amino acid sequence of SEQ ID NO: 19, the CDRL1 amino acid sequence of SEQ ID NO: 20, the CDRL2 amino acid sequence of SEQ ID NO: 24, and the CDRL3 amino acid sequence of SEQ ID NO: 22; (iii) the CDRH1 amino acid sequence of SEQ ID NO: 25, the CDRH2 amino acid sequence of SEQ ID NO: 26, the CDRH3 amino acid sequence of SEQ ID NO: 27, the CDRL1 amino acid sequence of SEQ ID NO: 28, the CDRL2 amino acid sequence of SEQ ID NO: 21, and the CDRL3 amino acid sequence of SEQ ID NO: 29; and (iv) the CDRH1 amino acid sequence of SEQ ID NO: 30, the CDRH2 amino acid sequence of SEQ ID NO: 31, the CDRH3 amino acid sequence of SEQ ID NO: 27, the CDRL1 amino acid sequence of SEQ ID NO: 28, the CDRL2 amino acid sequence of SEQ ID NO: 21, and the CDRL3 amino acid sequence of SEQ ID NO: 29.

In some embodiments, the antibodies disclosed herein contain the CDRH1 amino acid sequence of SEQ ID NO: 17, the CDRH2 amino acid sequence of SEQ ID NO: 18, the CDRH3 amino acid sequence of SEQ ID NO: 19, the CDRL1 amino acid sequence of SEQ ID NO: 20, the CDRL2 amino acid sequence of SEQ ID NO: 21, and the CDRL3 amino acid sequence of SEQ ID NO: 22.

In some embodiments, the antibodies disclosed herein contain the CDRH1 amino acid sequence of SEQ ID NO: 17, the CDRH2 amino acid sequence of SEQ ID NO: 23, the CDRH3 amino acid sequence of SEQ ID NO: 19, the CDRL1 amino acid sequence of SEQ ID NO: 20, the CDRL2 amino acid sequence of SEQ ID NO: 24, and the CDRL3 amino acid sequence of SEQ ID NO: 22.

In some embodiments, the antibodies disclosed herein contain the CDRH1 amino acid sequence of SEQ ID NO: 25, the CDRH2 amino acid sequence of SEQ ID NO: 26, the CDRH3 amino acid sequence of SEQ ID NO: 27, the CDRL1 amino acid sequence of SEQ ID NO: 28, the CDRL2 amino acid sequence of SEQ ID NO: 21, and the CDRL3 amino acid sequence of SEQ ID NO: 29.

In some embodiments, the antibodies disclosed herein contain the CDRH1 amino acid sequence of SEQ ID NO: 30, the CDRH2 amino acid sequence of SEQ ID NO: 31, the CDRH3 amino acid sequence of SEQ ID NO: 27, the CDRL1 amino acid sequence of SEQ ID NO: 28, the CDRL2 amino acid sequence of SEQ ID NO: 21, and the CDRL3 amino acid sequence of SEQ ID NO: 29.

In certain embodiments, the antibodies disclosed herein include one or more conservative amino acid substitutions in a variable domain sequence, such as SEQ ID NOs: 9-16, e.g., 1, 2, 3, 4, 5, 6, 7, 8, 9, 10, 11, 12, 13, 14, 15, or more conservative substitutions in a variable domain sequence. In some embodiments, these conservative amino acid substitutions are in a CDR region, e.g., 1, 2, 3, 4, 5, 6, 7, 8, 9, 10, 11, 12, 13, 14, 15, or more conservative substitutions are made cumulatively across all CDRs and in some particular embodiments, up to 1, 2, 3, or 4 conservative amino acid substitutions may be present in each CDR sequence, e.g., SEQ ID NOs: 17-31.

Those skilled in the art will recognize that it is possible to determine, without undue experimentation, if a monoclonal antibody has the same specificity as a monoclonal antibody disclosed herein (e.g., XMT 1517, XMT 1518, XMT 1519, and XMT 1520) by ascertaining whether the former prevents the latter from binding to a natural binding partner or other molecule known to be associated with HER2. If the monoclonal antibody being tested competes with the monoclonal antibody disclosed herein, as shown by a decrease in binding by the monoclonal antibody disclosed herein, then the two monoclonal antibodies bind to the same, or a closely related, epitope.

An alternative method for determining whether a monoclonal antibody has the specificity of monoclonal antibody disclosed herein is to pre-incubate the monoclonal antibody disclosed herein with soluble HER2 (with which it is normally reactive), and then add the monoclonal antibody being tested to determine if the monoclonal antibody being tested is inhibited in its ability to bind HER2. If the monoclonal antibody being tested is inhibited then, in all likelihood, it has the same, or functionally equivalent, epitopic specificity as the monoclonal antibody disclosed herein.

Screening of monoclonal antibodies disclosed herein, can be also carried out, e.g., by measuring HER2-mediated PI3K-Akt pathway activity, and determining whether the test monoclonal antibody is able to modulate, block, inhibit, reduce, antagonize, neutralize or otherwise interfere with PI3K-Akt pathway activity.

The HER2 antibodies suitable for the combinations or methods disclosed herein can be generated and purified by well-known techniques e.g. WO 2015/03643 1, incorporated herein in its entirety by reference.

### HER2 Antibody Conjugates

The invention pertains to combination therapies involving immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the trichothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as tolylene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies disclosed herein. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present disclosure, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

Preferred linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat.#21651G); (iv) Sulfo-LC-SPDP (sulfosuccinimidyl 6 [3-(2-pyridyldithio)-propianamide] hexanoate (Pierce Chem. Co. Cat.#2165-G); and (v) sulfo-NHS (N-hydroxysulfo-succinimide: Pierce Chem. Co., Cat.#24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

In some embodiments, the conjugate described herein includes a HER2 antibody or antigen-binding fragment thereof connected directly or indirectly to one or more D-carrying polymeric scaffolds independently comprising poly(1-hydroxymethylethylene hydroxymethyl-formal) (PHF) having a molecular weight ranging from about 2 kDa to about 40 kDa, wherein each of the one or more D-carrying polymeric scaffolds independently is of Formula (Ic):

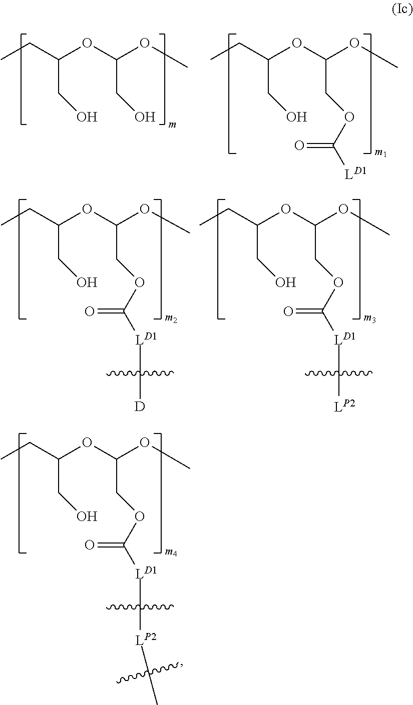

wherein:

each occurrence of D, independently, is a therapeutic or diagnostic agent;

L^{D1} is a carbonyl-containing moiety;

each occurrence of

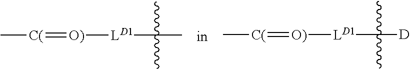

is independently a first linker that contains a biodegradable bond so that when the bond is broken, D is released in an active form for its intended therapeutic effect; and the

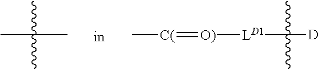

between L^{D1} and D denotes direct or indirect attachment of D to L^{D1};

each occurrence of

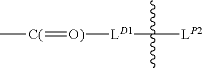

is independently a second linker not yet connected to the HER2 antibody or antigen-binding fragment thereof, in which L^{P2} is a moiety containing a functional group that is yet to form a covalent bond with a functional group of the antibody or antigen-binding fragment thereof, and the

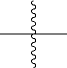

between L^{D1} and L^{P2} denotes direct or indirect attachment of L^{P2} to L^{D1}, and each occurrence of the second linker is distinct from each occurrence of the first linker;

each occurrence of

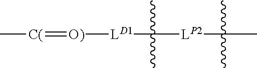

is independently a third linker that connects each D-carrying polymeric scaffold to the antibody or antigen-binding fragment thereof, in which the terminal

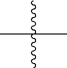

attached to L^{P2} denotes direct or indirect attachment of L^{P2} to the antibody or antigen-binding fragment thereof upon formation of a covalent bond between a functional group of L^{P2} and a functional group of the antibody or antigen-binding fragment thereof, and each occurrence of the third linker is distinct from each occurrence of the first linker;

m is an integer from 1 to about 300,

m₁ is an integer from 1 to about 140,

m₂ is an integer from 1 to about 40,

m₃ is an integer from 0 to about 18,

m₄ is an integer from 1 to about 10;

the sum of m, m₁, m₂, m₃, and m₄ ranges from about 15 to about 300; and the total number of L^{P2} attached to the antibody or antigen-binding fragment thereof is 10 or less.

The conjugate may include one or more of the following features.

For example, the HER2 antibody or antigen-binding fragment thereof is an isolated antibody or fragment thereof.

For example, in Formula (Ic), m₁ is an integer from 1 to about 120 (e.g., about 1-90) and/or m₃ is an integer from 1 to about 10 (e.g., about 1-8).

For example, when the PHF in Formula (Ic) has a molecular weight ranging from about 6 kDa to about 20 kDa (i.e., the sum of m, m₁, m₂, m₃, and m₄ ranging from about 45 to about 150), m₂ is an integer from 2 to about 20, m₃ is an integer from 0 to about 9, m₄ is an integer from 1 to about 10, and/or m₁ is an integer from 1 to about 75 (e.g., m₁ being about 4-45).

For example, when the PHF in Formula (Ic) has a molecular weight ranging from about 8 kDa to about 15 kDa (i.e., the sum of m, m₁, m₂, m₃, and m₄ ranging from about 60 to about 110), m₂ is an integer from 2 to about 15, m₃ is an integer from 0 to about 7, m₄ is an integer from 1 to about 10, and/or m₁ is an integer from 1 to about 55 (e.g., m₁ being about 4-30).

For example, when the PHF in Formula (Ic) has a molecular weight ranging from about 2 kDa to about 20 kDa (i.e., the sum of m, m₁, m₂, m₃, and m₄ ranging from about 15 to about 150), m₂ is an integer from 1 to about 20, m₃ is an integer from 0 to about 10 (e.g., m₃ ranging from 0 to about 9), m₄ is an integer from 1 to about 8, and/or m₁ is an integer from 1 to about 70, and the total number of L^{P2} connected to the antibody or antigen binding fragment thereof ranges from about 2 to about 8 (e.g., about 2, 3, 4, 5, 6, 7, or 8).

For example, when the PHF in Formula (Ic) has a molecular weight ranging from about 3 kDa to about 15 kDa (i.e., the sum of m, m₁, m₂, m₃, and m₄ ranging from about 20 to about 110), m₂ is an integer from 2 to about 15, m₃ is an integer from 0 to about 8 (e.g., m₃ ranging from 0 to about 7), m₄ is an integer from 1 to about 8, and/or m₁ is an integer from 2 to about 50, and the total number of L^{P2} connected to the antibody or antigen binding fragment thereof ranges from about 2 to about 8 (e.g., about 2, 3, 4, 5, 6, 7, or 8).

For example, when the PHF in Formula (Ic) has a molecular weight ranging from about 5 kDa to about 10 kDa, (i.e. the sum of m, m₁, m₂, m₃ and m₄ ranges from about 40 to about 75), m₂ is an integer from about 2 to about 10 (e.g., m₂ being about 3-10), m₃ is an integer from 0 to about 5 (e.g., m₃ ranging from 0 to about 4), m₄ is an integer from 1 to about 8 (e.g., m₄ ranging from 1 to about 5), and/or m₁ is an integer from about 2 to about 35 (e.g., m₁ being about 5-35), and the total number of L^{P2} connected to the antibody or antigen binding fragment thereof ranges from about 2 to about 8 (e.g., about 2, 3, 4, 5, 6, 7, or 8).

For example, when the PHF has a molecular weight ranging from 2 kDa to 40 kDa, (e.g., about 6-20 kDa or about 8-15 kDa, about 2-20 kDa, or about 3-15 kDa, or about 5-10 kDa), the number of drugs per PHF (e.g., m₂) is an integer from 1 to about 40 (e.g., about 1-20, or about 2-15 or about 3-10 or about 2-10). This scaffold can be used, for example, for conjugating the antibody or antigen-binding fragment thereof having a molecular weight of 40 kDa or greater (e.g., 60 kDa or greater; 80 kDa or greater; 100 kDa or greater; 120 kDa or greater; 140 kDa or greater; 160 kDa or greater, 180 kDa or greater, or 200 kDa or greater, or about 40-200 kDa, 40-180 kDa, 40-140 kDa, 60-200 kDa, 60-180 kDa, 60-140 kDa, 80-200 kDa, 80-180 kDa, 80-140 kDa, 100-200 kDa, 100-180 kDa, 100-140 kDa or 140-150 kDa). In this embodiment, the ratio of the antibody or antigen-binding fragment thereof to PHF is between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, between about 1:1 and about 1:2, between about 1:2 and about 1:4, between about 1:2 and about 1:3, between about 1:3 and about 1:4, or between about 1:3 and about 1:5.

For example, when the PHF has a molecular weight ranging from 2 kDa to 40 kDa, (e.g., about 6-20 kDa or about 8-15 kDa, about 2-20 kDa, or about 3-15 kDa, or about 5-10 kDa), the number of drugs per PHF (e.g., m₂) is an integer from 1 to about 40 (e.g., about 1-20, or about 2-15 or about 3-10 or about 2-10). This scaffold can be used, for example, for conjugating the antibody or antigen-binding fragment thereof having a molecular weight of 140 kDa to 180 kDa or of 140 kDa to 150 kDa. In this embodiment, the ratio of the antibody or antigen-binding fragment thereof to PHF is between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, between about 1:1 and about 1:2, between about 1:2 and about 1:4, between about 1:2 and about 1:3, between about 1:3 and about 1:4, or between about 1:3 and about 1:5.

The antibody or antigen-binding fragment thereof in this molecular weight range, include but are not limited to, for example, full length antibodies, such as, IgG, IgM.

For example, when the PHF has a molecular weight ranging from 2 kDa to 40 kDa, the number of drugs per PHF (e.g., m₂) is an integer from 1 to about 40 (e.g., about 1-20 or about 2-15 or about 3-10 or about 2-10). This scaffold can be used, for example, for conjugating the antibody or antigen-binding fragment thereof having a molecular weight of 60 kDa to 120 kDa. In this embodiment, the ratio of the antibody or antigen-binding fragment thereof to PHF is between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, between about 1:1 and about 1:2, between about 1:2 and about 1:4, between about 1:2 and about 1:3, between about 1:3 and about 1:4, or between about 1:3 and about 1:5.

The antibodies or antigen-binding fragments thereof in this molecular weight range, include but are not limited to, for example, antibody fragments such as, for example, Fab2 and camelids.

In certain embodiment, D is a therapeutic agent. In certain embodiments, the therapeutic agent is a small molecule having a molecular weight ≤ about 5 kDa, ≤ about 4 kDa, ≤ about 3 kDa, ≤ about 1.5 kDa, or ≤ about 1 kDa.

In certain embodiments, the therapeutic agent has an IC₅₀ of about less than 1 nM.

In another embodiment, the therapeutic agent has an IC₅₀ of about greater than 1 nM, for example, the therapeutic agent has an IC₅₀ of about 1 to 50 nM.

Some therapeutic agents having an IC₅₀ of greater than about 1 nM (e.g., “less potent drugs”) are unsuitable for conjugation with an antibody using art-recognized conjugation techniques. Without wishing to be bound by theory, such therapeutic agents have a potency that is insufficient for use in targeted antibody-drug conjugates using conventional techniques as sufficient copies of the drug (i.e., more than 8) cannot be conjugated using art-recognized techniques without resulting in diminished pharmacokinetic and physiochemical properties of the conjugate. However sufficiently high loadings of these less potent drugs can be achieved using the conjugation strategies described herein thereby resulting in high loadings of the therapeutic agent while maintaining the desirable pharmacokinetic and physiochemical properties. Thus, the disclosure also relates to an antibody-polymer-drug conjugate which includes the antibody or antigen-binding fragment thereof, PHF and at least eight therapeutic agent moieties, where D is auristatin, Dolastatin, monomethylauristatin E (MMAE), monomethylauristatin F (MMAF), auristatin F, AF HPA, MMAF HPA, or phenylenediamine (AFP).

For example, the duocarmycin or analogs thereof is duocarmycin A, duocarmycin B1, duocarmycin B2, duocarmycin C₁, duocarmycin C2, duocarmycin D, duocarmycin SA, CC-1065, adozelesin, bizelesin, or carzelesin.

Other examples of D include those described in, for example, US Application Publication No. 2013-0101546 and U.S. Pat. No. 8,815,226; and co-pending applications with U.S. Ser. Nos. 14/512,316 filed Oct. 10, 2014, 61/988,011 filed May 2, 2014, and 62/010,972 filed Jun. 11, 2014; the disclosure of each of which is incorporated herein in its entirety.

In some embodiments, the number of D-carrying polymeric scaffolds that can be conjugated to an antibody is limited by the number of free cysteine residues. In some embodiments, free cysteine residues are introduced into the antibody amino acid sequence by the methods described herein. Exemplary conjugates disclosed herein can include antibodies that have 1, 2, 3, or 4 engineered cysteine amino acids (Lyon, R. et al (2012) Methods in Enzym. 502:123-138). In some embodiments, one or more free cysteine residues are already present in an antibody, without the use of engineering, in which case the existing free cysteine residues may be used to conjugate the antibody to a D-carrying polymeric scaffold. In some embodiments, an antibody is exposed to reducing conditions prior to conjugation of the antibody in order to generate one or more free cysteine residues.

In certain embodiments, in the conjugate described herein, the D-carrying polymeric scaffold of Formula (Ic) is of Formula (Ie):

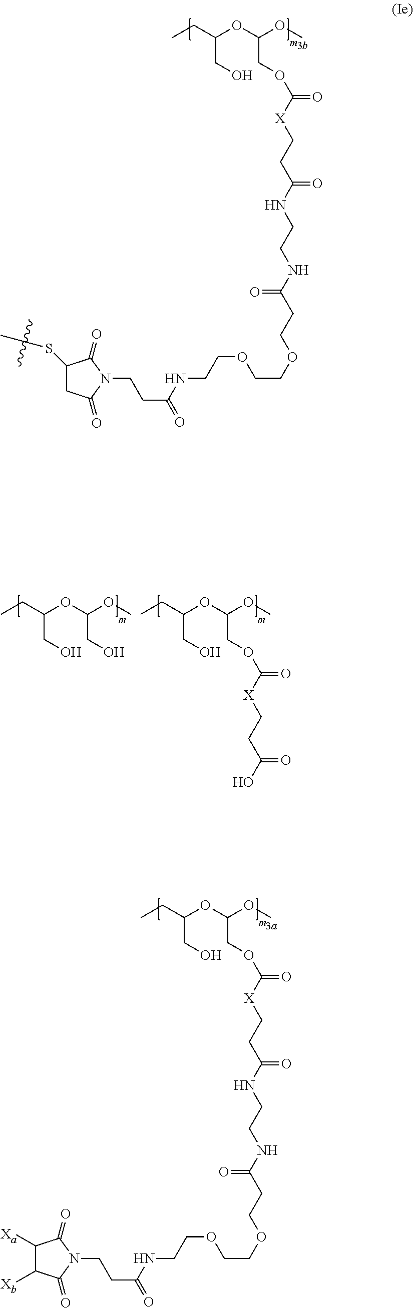

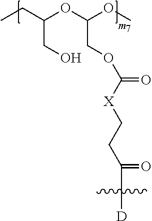

wherein,

the PHF has a molecular weight ranging from about 2 kDa to about 40 kDa;

each occurrence of D independently is a therapeutic agent having a molecular weight of ≤5 kDa, and the

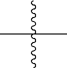

between D and the carbonyl group denotes direct or indirect attachment of D to the carbonyl group,

X is CH₂, O, or NH;

one of Xₐ and X_{b} is H and the other is a water-soluble maleimido blocking moiety, or Xₐ and X_{b}, together with the carbon atoms to which they are attached for a carbon-carbon double bond, m₁ is an integer from 1 to about 140,

m₇ is an integer from 1 to about 40, and the sum of m₁ and m₇ is about 2 to about 180

m is an integer from 1 to about 300,

m₃ₐ is an integer from 0 to about 17,

m_{3b} is an integer from 1 to about 8, and the sum of m₃ₐ and m_{3b} is between 1 and 18, and

the sum of m, m₁, m₇, m₃ₐ, and m_{3b} ranges from about 15 to about 300.

In certain embodiments, in the conjugate described herein, the D-carrying polymeric scaffold of Formula (Ie) is of Formula (Id):

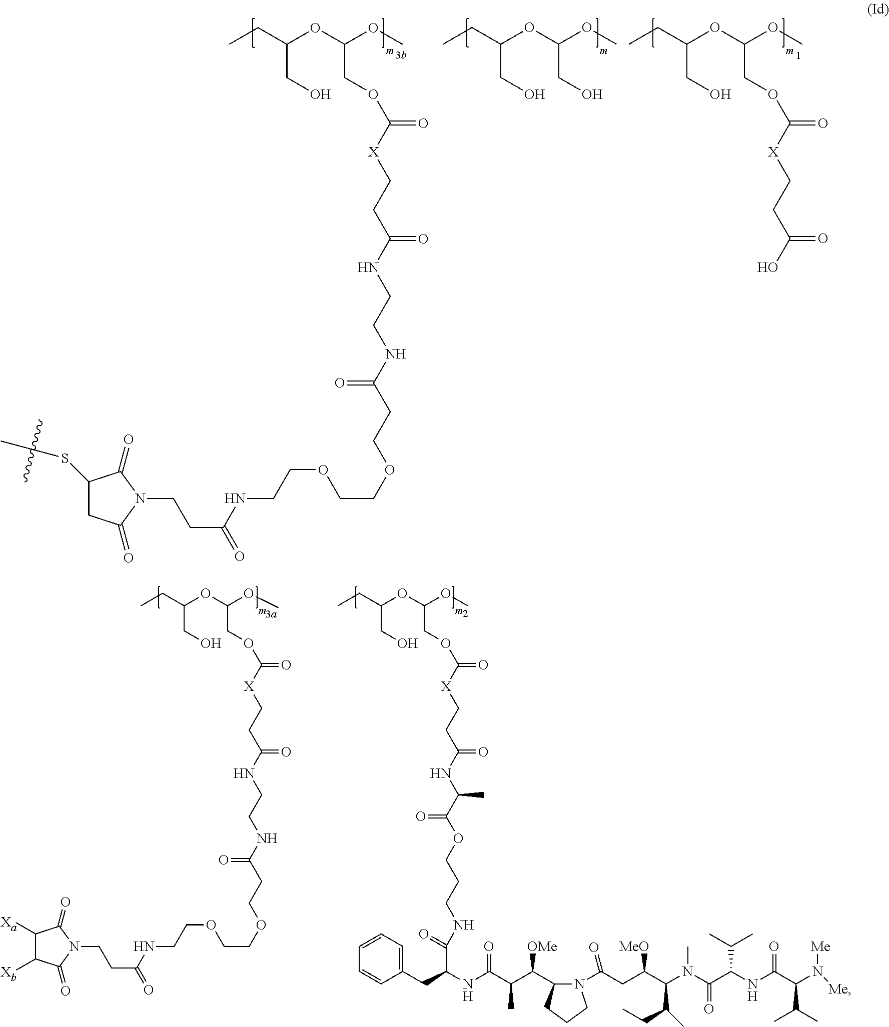

wherein:

one of Xₐ and X_{b} is H and the other is a water-soluble maleimido blocking moiety, or Xₐ and X_{b}, together with the carbon atoms to which they are attached for a carbon-carbon double bond;

m₃ₐ is an integer from 0 to about 17,

m_{3b} is an integer from 1 to about 8, and the sum of m₃ₐ and m_{3b} is between 1 and 18, and

the sum of m, m₁, m₂, m₃ₐ, and m_{3b} ranges from about 15 to about 300.

In certain embodiments, in the conjugate described herein, the D-carrying polymeric scaffold of Formula (Ie) is of Formula (Id-1):

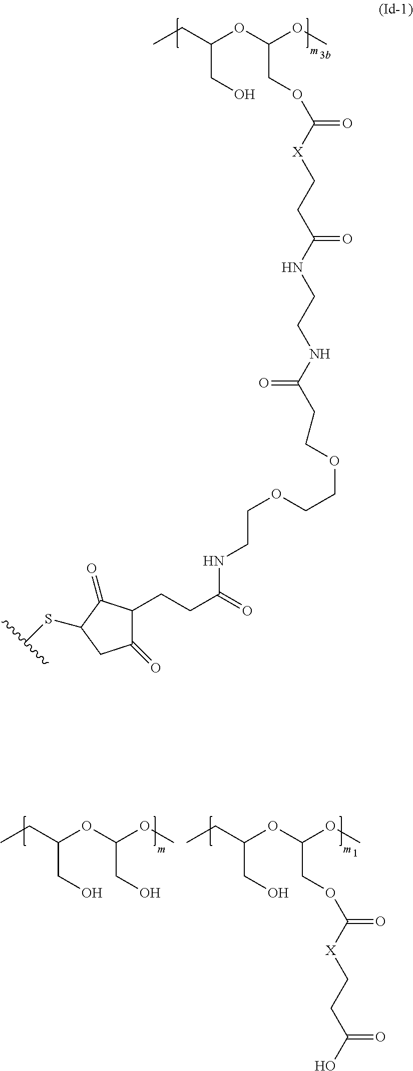

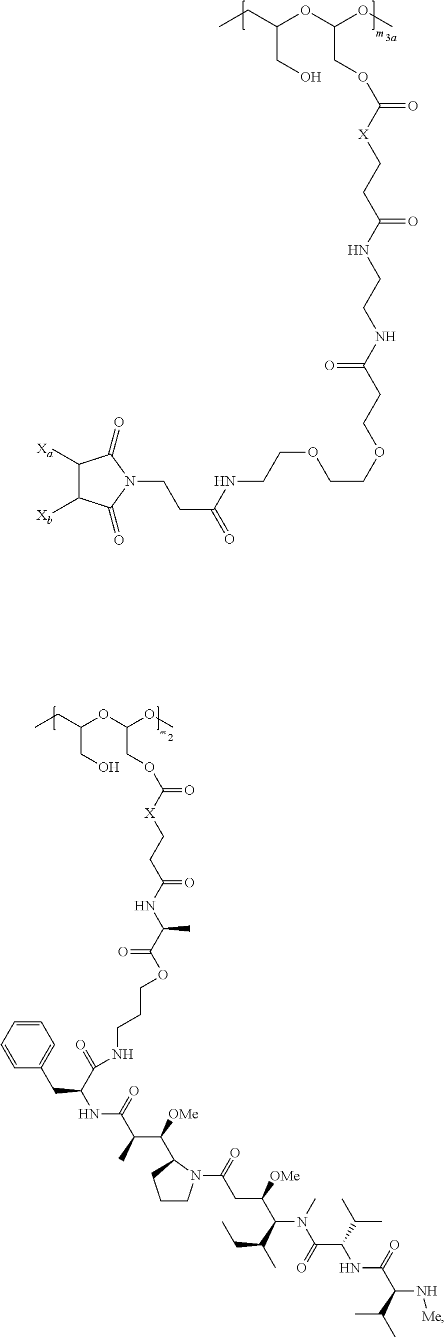

wherein:

one of Xₐ and X_{b} is H and the other is a water-soluble maleimido blocking moiety, or Xₐ and X_{b}, together with the carbon atoms to which they are attached for a carbon-carbon double bond;

m₃ₐ is an integer from 0 to about 17,

m_{3b} is an integer from 1 to about 8, and the sum of m₃ₐ and m_{3b} is between 1 and 18, and

the sum of m, m₁, m₂, m₃ₐ, and m_{3b} ranges from about 15 to about 300.

For example, the ratio between m₂ and m_{3b} is greater than 1:1 and less than or equal to 10:1.

For example, the ratio between m₂ and m_{3b} is about 9:1, 8:1, 7:1, 6:1, 5:1, 4:1, 3:1, or 2:1.

For example, the ratio between m₂ and m_{3b} is between 2:1 and 8:1.

For example, the ratio between m₂ and m_{3b} is about 8:1, 7:1, 6:1, 5:1, 4:1, 3:1, or 2:1.

For example, the ratio between m₂ and m_{3b} is between 2:1 and 4:1.

For example, the ratio between m₂ and m_{3b} is about 4:1, 3:1, or 2:1.

For example, the ratio between m₂ and m_{3b} is about 3:1 and 5:1.

For example, the ratio between m₂ and m_{3b} is about 3:1, 4:1 or 5:1.

For example, when the PHF in Formula (Id) or (Id-1) has a molecular weight ranging from about 2 kDa to about 20 kDa, the sum of m, m₁, m₂, m₃ₐ and m_{3b} ranges from about 15 to about 150, m₁ is an integer from 1 to about 70, m₂ is an integer from 1 to about 20, m₃ₐ is an integer from 0 to about 9, m_{3b} is an integer from 1 to about 8 and the ratio between the PHF and the HER2 antibody or antigen-binding fragment thereof is an integer from 2 to about 8.

For example, when the PHF in Formula (Id) or (Id-1) has a molecular weight ranging from about 3 kDa to about 15 kDa, the sum of m, m₁, m₂, m₃ₐ and m_{3b} ranges from about 20 to about 110, m₁ is an integer from 2 to about 50, m₂ is an integer from 2 to about 15, m₃ₐ is an integer from 0 to about 7, m_{3b} is an integer from 1 to about 8 and the ratio between the PHF and the HER2 antibody or antigen-binding fragment thereof is an integer from 2 to about 8 (e.g., an integer from 2 to about 6 or an integer from 2 to about 4).

For example, when the PHF in Formula (Id) or (Id-1) has a molecular weight ranging from about 5 kDa to about 10 kDa, the sum of m, m₁, m₂, m₃ₐ and m_{3b} ranges from about 40 to about 75, m₁ is an integer from about 2 to about 35, m₂ is an integer from about 2 to about 10, m₃ₐ is an integer from 0 to about 4, m_{3b} is an integer from 1 to about 5 and the ratio between the PHF and the HER2 antibody or antigen-binding fragment thereof is an integer from 2 to about 8 (e.g., an integer from 2 to about 6 or an integer from 2 to about 4).

For example, the water-soluble maleimido blocking moieties are moieties that can be covalently attached to one of the two olefin carbon atoms upon reaction of the maleimido group with a thiol-containing compound of Formula (II):

R₉₀—(CH₂)_{d}—SH (II)

wherein:

R₉₀ is NHR₉₁, OH, COOR₉₃, CH(NHR₉₁)COOR₉₃ or a substituted phenyl group;

R₉₃ is hydrogen or C₁₋₄ alkyl;

R₉₁ is hydrogen, CH₃ or CH₃CO and

d is an integer from 1 to 3.

For example, the water-soluble maleimido blocking compound of Formula (II) can be cysteine, N-acetyl cysteine, cysteine methyl ester, N-methyl cysteine, 2-mercaptoethanol, 3-mercaptopropanoic acid, 2-mercaptoacetic acid, mercaptomethanol (i.e., HOCH₂SH), benzyl thiol in which phenyl is substituted with one or more hydrophilic substituents, or 3-aminopropane-1-thiol. The one or more hydrophilic substituents on phenyl comprise OH, SH, methoxy, ethoxy, COOH, CHO, COC₁₋₄ alkyl, NH₂, F, cyano, SO₃H, PO₃H, and the like.

For example, the water-soluble maleimido blocking group is —S—(CH₂)_{d}—R₉₀, in which,

R₉₀ is OH, COOH, or CH(NHR₉₁)COOR₉₃;
R₉₃ is hydrogen or CH₃;
R₉₁ is hydrogen or CH₃CO; and
d is 1 or 2.

For example, the water-soluble maleimido blocking group is —S—CH₂—CH(NH₂)COOH.

For example, when the PHF has a molecular weight ranging from 2 kDa to 40 kDa, (e.g., about 2-20 kDa, or about 3-15 kDa, or about 5-10 kDa), the number of drugs per PHF (e.g., m₂) is an integer from 1 to about 40 (e.g., about 1-20 or about 2-15 or about 3-10 or about 2-10). This scaffold can be used, for example, for conjugating an antibody or antigen-binding fragment thereof having a molecular weight of 40 kDa or greater (e.g., 60 kDa or greater; 80 kDa or greater; or 100 kDa or greater; 120 kDa or greater; 140 kDa or greater; 160 kDa or greater, 180 kDa or greater, or 200 kDa or greater, or about 40-200 kDa, 40-180 kDa, 40-140 kDa, 60-200 kDa, 60-180 kDa, 60-140 kDa, 80-200 kDa, 80-180 kDa, 80-140 kDa, 100-200 kDa, 100-180 kDa, 100-140 kDa or 140-150 kDa). In this embodiment, the ratio of the antibody or antigen-binding fragment thereof to PHF is between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, between about 1:1 and about 1:2, between about 1:2 and about 1:8, between about 1:2 and about 1:6, between about 1:2 and about 1:5, between about 1:2 and about 1:4, between about 1:2 and about 1:3, between about 1:3 and about 1:4, or between about 1:3 and about 1:5.

For example, when the PHF has a molecular weight ranging from 2 kDa to 40 kDa, (e.g., about 2-20 kDa, or about 3-15 kDa, or about 5-10 kDa), the number of drugs per PHF (e.g., m₂) is an integer from 1 to about 40 (e.g., about 1-20 or about 2-15 or about 3-10 or about 2-10). This scaffold can be used, for example, for conjugating an antibody or antigen-binding fragment having a molecular weight of 140 kDa to 180 kDa or of 140 kDa to 150 kDa. In this embodiment the ratio of the antibody or antigen-binding fragment thereof to PHF is between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, between about 1:1 and about 1:2, between about 1:2 and about 1:8, between about 1:2 and about 1:6, between about 1:2 and about 1:5, between about 1:2 and about 1:4, between about 1:2 and about 1:3, between about 1:3 and about 1:4, or between about 1:3 and about 1:5.

The antibodies or antigen-binding fragments in this molecular weight range, include but are not limited to, for example, full length antibodies, such as, IgG, IgM.

For example, when the PHF has a molecular weight ranging from 2 kDa to 40 kDa (e.g., about 2-20 kDa, or about 3-15 kDa, or about 5-10 kDa), the number of drugs per PHF (e.g., m₂) is an integer from 1 to about 40, (e.g., about 1-20 or about 2-15 or about 3-10 or 2-10). This scaffold can be used, for example, for conjugating an antibody or antigen-binding fragment having a molecular weight of 60 kDa to 120 kDa. In this embodiment the ratio of the antibody or antigen-binding fragment thereof to PHF is between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, between about 1:1 and about 1:2, between about 1:2 and about 1:8, between about 1:2 and about 1:6, between about 1:2 and about 1:5, between about 1:2 and about 1:4, between about 1:2 and about 1:3, between about 1:3 and about 1:4, or between about 1:3 and about 1:5.

The antibodies or antigen-binding fragments in this molecular weight range, include but are not limited to, for example, antibody fragments such as, for example Fab2, scFcFv and camelids.

For example, when the PHF has a molecular weight ranging from 2 kDa to 40 kDa, (e.g., about 2-20 kDa, or about 3-15 kDa, or about 5-10 kDa), the number of drugs per PHF (e.g., m₂) is an integer from 1 to about 40 (e.g., about 1-20 or about 2-15 or about 3-10 or 2-10). This scaffold can be used, for example, for conjugating the antibody or antigen-binding fragment thereof having a molecular weight of 40 kDa to 80 kDa. In this embodiment the ratio of the antibody or antigen-binding fragment thereof to PHF is between about 1:1 and about 1:10, between about 1:1 and about 1:9, between about 1:1 and about 1:8, between about 1:1 and about 1:7, between about 1:1 and about 1:6, between about 1:1 and about 1:5, between about 1:1 and about 1:4, between about 1:1 and about 1:3, between about 1:1 and about 1:2, between about 1:2 and about 1:8, between about 1:2 and about 1:6, between about 1:2 and about 1:5, between about 1:2 and about 1:4, between about 1:2 and about 1:3, between about 1:3 and about 1:4, or between about 1:3 and about 1:5.

The antibodies or antigen-binding fragments in this molecular weight range, i.e., about 40 kDa to about 80 kDa, include but are not limited to, for example, antibody fragments such as, for example, Fabs.

In certain embodiments, in the conjugate described herein, the D-carrying polymeric scaffold of Formula (Ie) is of Formula (If), wherein the polymer is PHF that has a molecular weight ranging from about 2 kDa to about 40 kDa:

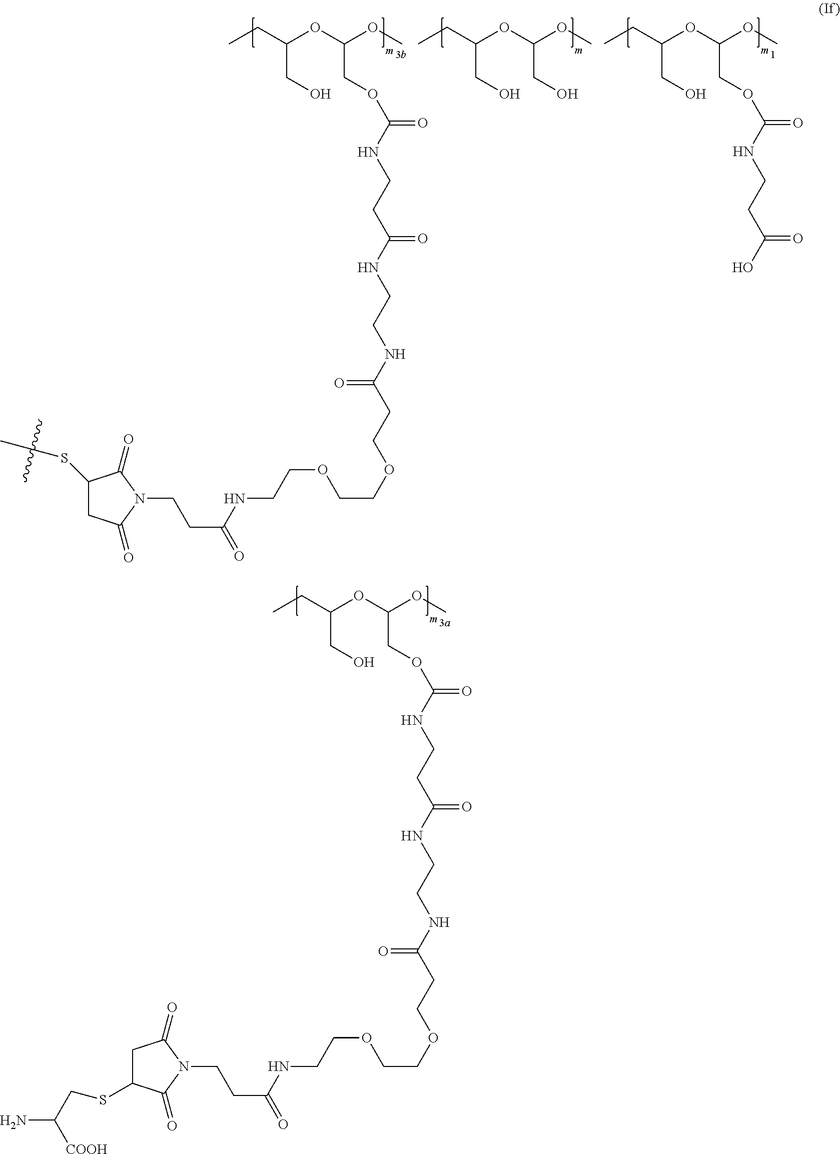

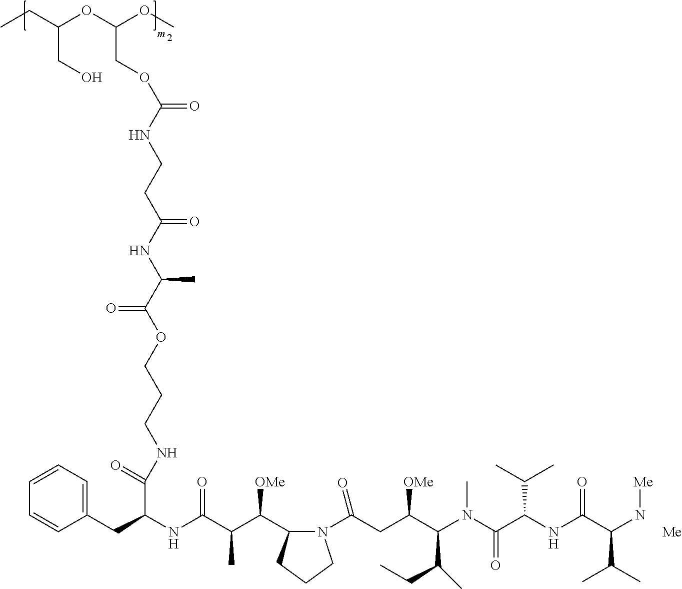

wherein:

m is an integer from 1 to about 300,

m₁ is an integer from 1 to about 140,

m₂ is an integer from 1 to about 40,

m₃ₐ is an integer from 0 to about 17,

m_{3b} is an integer from 1 to about 8;

the sum of m₃ₐ and m_{3b} ranges from 1 and about 18;

the sum of m, m₁, m₂, m₃ₐ, and m_{3b} ranges from about 15 to about 300;

the terminal

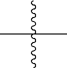

denotes the attachment of one or more polymeric scaffolds to the antibody or antigen binding fragment thereof that specifically binds to an epitope of the human HER2 receptor and comprises a variable heavy chain complementarity determining region 1 (CDRH1) comprising the amino acid sequence FTFSSYSMN (SEQ ID NO: 25); a variable heavy chain complementarity determining region 2 (CDRH2) comprising the amino acid sequence YISSSSSTIYYADSVKG (SEQ ID NO: 26); a variable heavy chain complementarity determining region 3 (CDRH3) comprising the amino acid sequence GGHGYFDL (SEQ ID NO: 27); a variable light chain complementarity determining region 1 (CDRL1) comprising the amino acid sequence RASQSVSSSYLA (SEQ ID NO: 28); a variable light chain complementarity determining region 2 (CDRL2) comprising the amino acid sequence GASSRAT (SEQ ID NO: 21); and a variable light chain complementarity determining region 3 (CDRL3) comprising the amino acid sequence QQYHHSPLT (SEQ ID NO: 29); and

the ratio between the PHF and the antibody is 10 or less.

The scaffold of Formula (If) can include one or more of the following features:

When the PHF in Formula (If) has a molecular weight ranging from about 2 kDa to about 20 kDa, the sum of m, m₁, m₂, m₃ₐ and m_{3b} ranges from about 15 to about 150, m₁ is an integer from 1 to about 70, m₂ is an integer from 1 to about 20, m₃ₐ is an integer from 0 to about 9, m_{3b} is an integer from 1 to about 8, the sum of m₃ₐ and m_{3b} ranges from 1 and about 10, and the ratio between the PHF and antibody is an integer from 2 to about 8 (e.g., from about 2 to about 6 or from about 2 to about 4).

When the PHF in Formula (If) has a molecular weight ranging from about 3 kDa to about 15 kDa, the sum of m, m₁, m₂, m₃ₐ and m_{3b} ranges from about 20 to about 110, m₁ is an integer from 2 to about 50, m₂ is an integer from 2 to about 15, m₃ₐ is an integer from 0 to about 7, m_{3b} is an integer from 1 to about 8, the sum of m₃ₐ and m_{3b} ranges from 1 and about 8; and the ratio between the PHF and antibody is an integer from 2 to about 8 (e.g., from about 2 to about 6 or from about 2 to about 4).

When the PHF in Formula (If) has a molecular weight ranging from about 5 kDa to about 10 kDa, the sum of m, m₁, m₂, m₃ₐ and m_{3b} ranges from about 40 to about 75, m₁ is an integer from about 2 to about 35, m₂ is an integer from about 2 to about 10, m₃ₐ is an integer from 0 to about 4, m_{3b} is an integer from 1 to about 5, the sum of m₃ₐ and m_{3b} ranges from 1 and about 5; and the ratio between the PHF and antibody is an integer from 2 to about 8 (e.g., from about 2 to about 6 or from about 2 to about 4).

In certain embodiments, the ratio between auristatin F hydroxypropyl amide (“AF HPA”) and the antibody ranges from about 30:1 to about 6:1 (e.g., about 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1 or 6:1).

In certain embodiments, the ratio between AF HPA and the antibody ranges from about 25:1 to about 6:1 (e.g., about 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1 or 6:1).

In other embodiments, the ratio between AF HPA and the antibody ranges from about 20:1 to about 6:1 (e.g., about 20:1, 19:1, 18:1, 17:1, 16:1, 15:1, 14:1, 13:1, 12:1, 11:1, 10:1, 9:1, 8:1, 7:1 or 6:1).

In some embodiments, the ratio between AF HPA and the antibody ranges from about 16:1 to about 9:1 (e.g., about 16:1, 15:1, 14:1, 13:1, 12:1, 11:1, 10:1 or 9:1).

In some embodiments, the ratio between AF and the antibody ranges from about 15:1 to about 9:1 (e.g., about 15:1, 14:1, 13:1, 12:1, 11:1, 10:1 or 9:1).

In some embodiments, the ratio between AF HPA and the antibody ranges from about 15:1 to about 10:1 (e.g., about 15:1, 14:1, 13:1, 12:1, 11:1 or 10:1).

In some embodiments, the ratio between AF HPA and the antibody ranges from about 15:1 to about 12:1 (e.g., about 15:1, 14:1, 13:1, 12:1).

In some embodiments, the ratio between AF HPA and the antibody ranges from about 12:1 to about 9:1 (e.g., about 12:1, 11:1, 10:1 or 9:1).

In certain embodiments, the ratio between PHF and the antibody ranges from about 10:1 to about 1:1 (e.g., about 10:1, 9:1, 8:1, 7:1, 6:1, 5:1, 4:1, 3:1, 2:1 or 1:1).

In certain embodiments, the ratio between PHF and the antibody ranges from about 8:1 to about 2:1 (e.g., about 8:1, 7:1, 6:1, 5:1, 4:1, 3:1 or 2:1).

In other embodiments, the ratio between PHF and the antibody ranges from about 6:1 to about 1:1 (e.g., about 6:1, 5:1, 4:1, 3:1, 2:1 or 1:1).

In other embodiments, the ratio between PHF and the antibody ranges from about 6:1 to about 2:1 (e.g., about 6:1, 5:1, 4:1, 3:1 or 2:1).

In other embodiments, the ratio between PHF and the antibody ranges from about 6:1 to about 3:1 (e.g., about 6:1, 5:1, 4:1 or 3:1).

In other embodiments, the ratio between PHF and the antibody ranges from about 5:1 to about 2:1 (e.g., about 5:1, 4:1, 3:1 or 2:1).

In some embodiments, the ratio between PHF and the antibody ranges from about 5:1 to about 3:1 (e.g., about 5:1, 4:1 or 3:1).

In some embodiments, the ratio between PHF and the antibody ranges from about 4:1 to about 2:1 (e.g., about 4:1, 3:1 or 2:1).

The antibodies or antigen-binding fragments in this molecular weight range, include but are not limited to, for example, antibody fragments, such as, Fabs.

In certain embodiments, in the conjugate described herein, the D-carrying polymeric scaffold of Formula (If) is of Formula (Ig), wherein the polymer is PHF that has a molecular weight ranging from about 5 kDa to about 10 kDa:

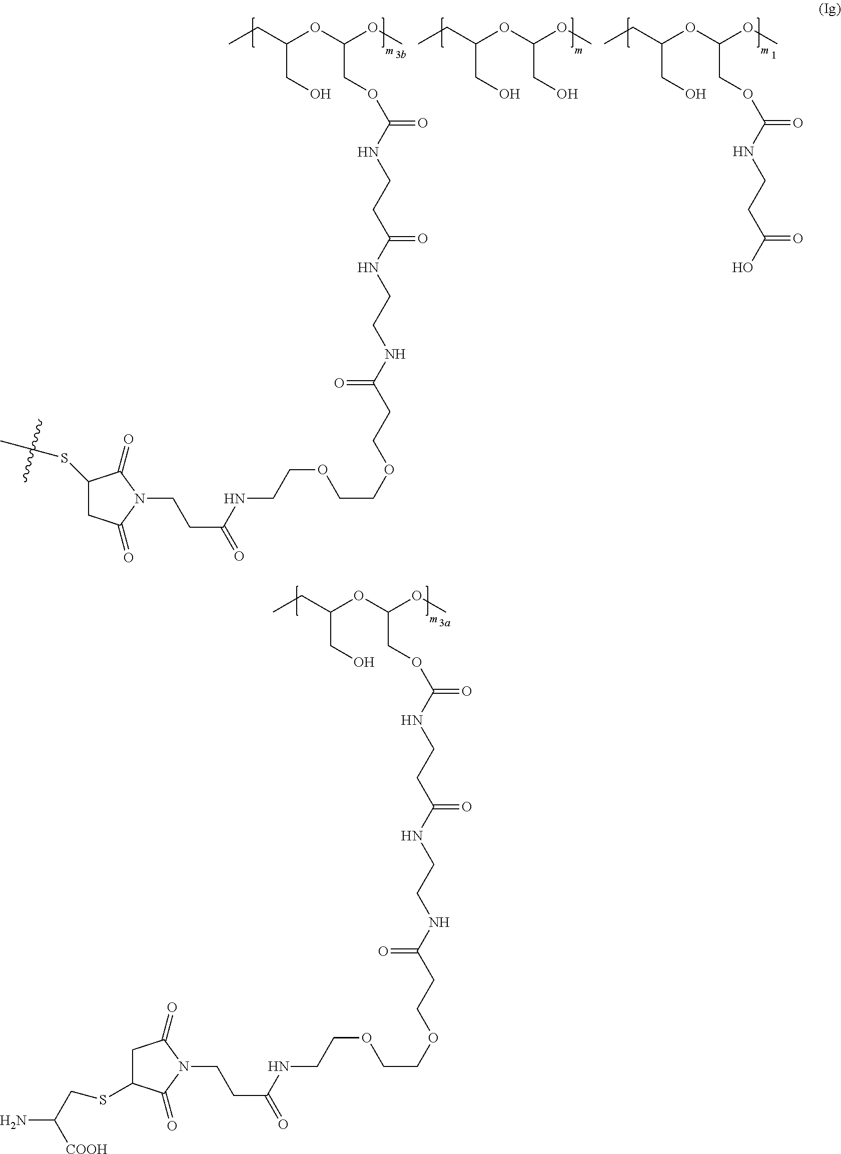

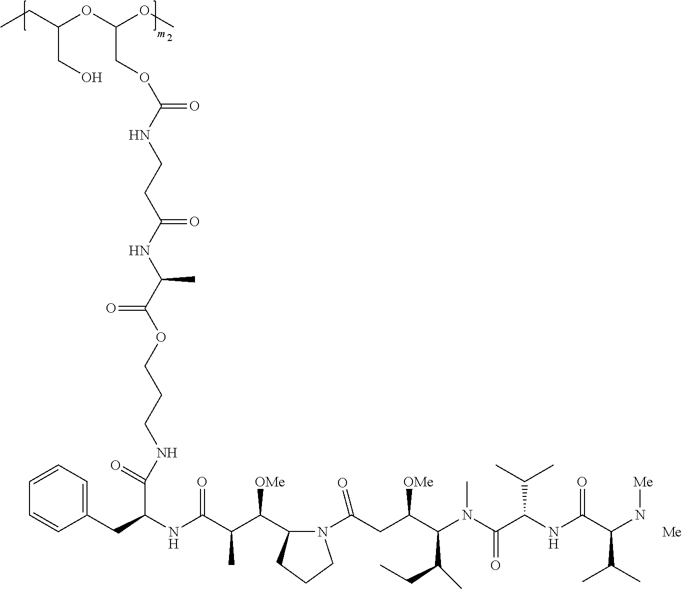

wherein:

m is an integer from 30 to about 35,

m₁ is an integer from 8 to about 10,

m₂ is an integer from 2 to about 5,

m₃ₐ is an integer from 0 to about 1,

m_{3b} is an integer from 1 to about 2;

the sum of m₃ₐ and m_{3b} ranges from 1 and about 4;

the terminal

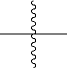

denotes the attachment of one or more polymeric scaffolds to the antibody or antigen binding fragment thereof that specifically binds to an epitope of the human HER2 receptor and comprises a variable heavy chain complementarity determining region 1 (CDRH1) comprising the amino acid sequence FTFSSYSMN (SEQ ID NO: 25); a variable heavy chain complementarity determining region 2 (CDRH2) comprising the amino acid sequence YISSSSSTIYYADSVKG (SEQ ID NO: 26); a variable heavy chain complementarity determining region 3 (CDRH3) comprising the amino acid sequence GGHGYFDL (SEQ ID NO: 27); a variable light chain complementarity determining region 1 (CDRL1) comprising the amino acid sequence RASQSVSSSYLA (SEQ ID NO: 28); a variable light chain complementarity determining region 2 (CDRL2) comprising the amino acid sequence GASSRAT (SEQ ID NO: 21); and a variable light chain complementarity determining region 3 (CDRL3) comprising the amino acid sequence QQYHHSPLT (SEQ ID NO: 29); and the ratio between the PHF and the antibody is about 3 to about 5.

Other embodiments of antibody-polymer drug conjugates are those described in, for example, U.S. Pat. No. 8,815,226; U.S. Pat. No. 9,849,191, and U.S. Pat. No. 9,555,122; the disclosure of each of which is incorporated herein in its entirety.

This disclosure also relates to a drug derivative so modified that it can be directly conjugated to an antibody or antigen-binding fragment thereof absent a polymeric carrier, and the drug-antibody conjugates thereof.

In some embodiments, the antibody-drug conjugates include an antibody or antigen-binding fragment thereof conjugated, i.e. covalently attached, to the drug moiety. In some embodiments, the antibody or antigen-binding fragment thereof is covalently attached to the drug moiety through a linker, e.g., a non-polymeric linker.

The drug moiety (D) of the antibody-drug conjugates (ADC) may include any compound, moiety or group that has a cytotoxic or cytostatic effect as defined herein. In certain embodiments, an antibody-drug conjugate (ADC) comprises an antibody (Ab) which targets a tumor cell, a drug moiety (D), and a linker moiety (L) that attaches Ab to D. In some embodiments, the antibody is attached to the linker moiety (L) through one or more amino acid residues, such as lysine and/or cysteine.

In certain embodiments the ADC has Formula (Ig):

Ab-(L-D)ₚ (Ig),

where p is 1 to about 20.

In some embodiments, the number of drug moieties that can be conjugated to an antibody is limited by the number of free cysteine residues. In some embodiments, free cysteine residues are introduced into the antibody amino acid sequence by the methods described herein. Exemplary ADC of Formula Ig include, but are not limited to, antibodies that have 1, 2, 3, or 4 engineered cysteine amino acids (Lyon, R. et al (2012) Methods in Enzym. 502:123-138). In some embodiments, one or more free cysteine residues are already present in an antibody, without the use of engineering, in which case the existing free cysteine residues may be used to conjugate the antibody to a drug. In some embodiments, an antibody is exposed to reducing conditions prior to conjugation of the antibody in order to generate one or more free cysteine residues.

In some embodiments the “Linker” (L) is a bifunctional or multifunctional moiety that can be used to link one or more drug moieties (D) to an antibody (Ab) to form an antibody-drug conjugate (ADC) of Formula Ig. In some embodiments, antibody-drug conjugates (ADC) can be prepared using a Linker having reactive functionalities for covalently attaching to the drug and to the antibody. For example, in some embodiments, a cysteine thiol of an antibody (Ab) can form a bond with a reactive functional group of a linker or a drug-linker intermediate to make an ADC.

In one aspect, a linker has a functionality that is capable of reacting with a free cysteine present on an antibody to form a covalent bond. Nonlimiting exemplary such reactive functionalities include maleimide, haloacetamides, a-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a linker has a functionality that is capable of reacting with an electrophilic group present on an antibody. Exemplary such electrophilic groups include, but are not limited to, aldehyde and ketone carbonyl groups. In some embodiments, a heteroatom of the reactive functionality of the linker can react with an electrophilic group on an antibody and form a covalent bond to an antibody unit. Nonlimiting exemplary such reactive functionalities include, but are not limited to, hydrazide, oxime, amino, hydrazine, thiosemicarbazone, hydrazine carboxylate, and aryl hydrazide.

A linker may comprise one or more linker components. Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl)cyclohexane-1 carboxylate (“MCC”). Various linker components are known in the art, some of which are described below.

A linker may be a “cleavable linker,” facilitating release of a drug. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020).

In certain embodiments, a linker has the following Formula (IIg):

-Aₐ-W_{w}—Y_{y} (IIg)

wherein:

A is a “stretcher unit”, and a is an integer from 0 to 1;

W is an “amino acid unit”, and w is an integer from 0 to 12;

Y is a “spacer unit”, and y is an integer 0, 1, or 2. An ADC comprising the linker of Formula (IIg) has the Formula I(A): Ab-(Aa-Ww-Yy-D)p, wherein Ab, D, and p are defined as above for Formula (Ig). Exemplary embodiments of such linkers are described in U.S. Pat. No. 7,498,298, which is incorporated herein by reference in its entirety.

In some embodiments, a linker component comprises a “stretcher unit” (A) that links an antibody to another linker component or to a drug moiety. Nonlimiting exemplary stretcher units are shown below (wherein the wavy line indicates sites of covalent attachment to an antibody, drug, or additional linker components):

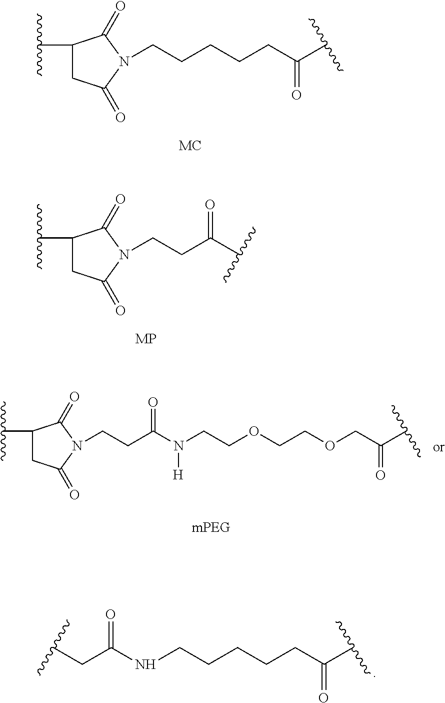 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component comprises an “amino acid unit” (W). In some such embodiments, the amino acid unit allows for cleavage of the linker by a protease, thereby facilitating release of the drug from the immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, cathepsin B, C and D, or a plasmin protease.

Typically, peptide-type linkers can be prepared by forming a peptide bond between two or more amino acids and/or peptide fragments. Such peptide bonds can be prepared, for example, according to a liquid phase synthesis method (e.g., E. Schrider and K. Lubke (1965) “The Peptides”, volume 1, pp 76-136, Academic Press).

In some embodiments, a linker component comprises a “spacer” unit that links the antibody to a drug moiety, either directly or through a stretcher unit and/or an amino acid unit. A spacer unit may be “self-immolative” or a “non-self-immolative.” A “non-self-immolative” spacer unit is one in which part or all of the spacer unit remains bound to the drug moiety upon cleavage of the ADC. Examples of non-self-immolative spacer units include, but are not limited to, a glycine spacer unit and a glycine-glycine spacer unit. In some embodiments, enzymatic cleavage of an ADC containing a glycine-glycine spacer unit by a tumor-cell associated protease results in release of a glycine-glycine-drug moiety from the remainder of the ADC. In some such embodiments, the glycine-glycine-drug moiety is subjected to a hydrolysis step in the tumor cell, thus cleaving the glycine-glycine spacer unit from the drug moiety.

A “self-immolative” spacer unit allows for release of the drug moiety. In certain embodiments, a spacer unit of a linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the spacer unit comprises p-aminobenzyloxycarbonyl (PAB). In some embodiments, an ADC comprising a self-immolative linker has the structure:

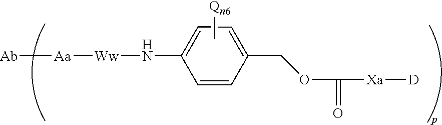

wherein:
Q is —C₁-C₈ alkyl, —O—(C₁-C₈ alkyl), halogen, nitro, or cyano;
n₆ is an integer from 0 to 4;
Xₐ may be one or more additional spacer units or may be absent; and
p in an integer from 1 to about 20.

In some embodiments, p in an integer from 1 to 10, 1 to 7, 1 to 5, or 1 to 4.

Nonlimiting exemplary Xₐ spacer units include:

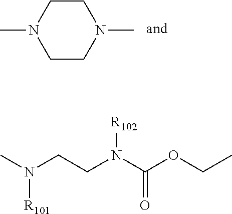

wherein R₁₀₁ and R₁₀₂ are independently selected from H and C₁-C₆ alkyl. In some embodiments, R₁₀₁ and R₁₀₂ are each —CH₃.

Other examples of self-immolative spacers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Linkage of a drug to the a-carbon of a glycine residue is another example of a self-immolative spacer that may be useful in ADC (Kingsbury et al (1984) J. Med. Chem. 27:1447).

In some embodiments, linker L may be a dendritic type linker for covalent attachment of more than one drug moiety to an antibody through a branching, multifunctional linker moiety (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where an antibody bears only one reactive cysteine thiol group, a multitude of drug moieties may be attached through a dendritic linker.

Nonlimiting exemplary linkers are shown below for ADCs of Formula (Ig):

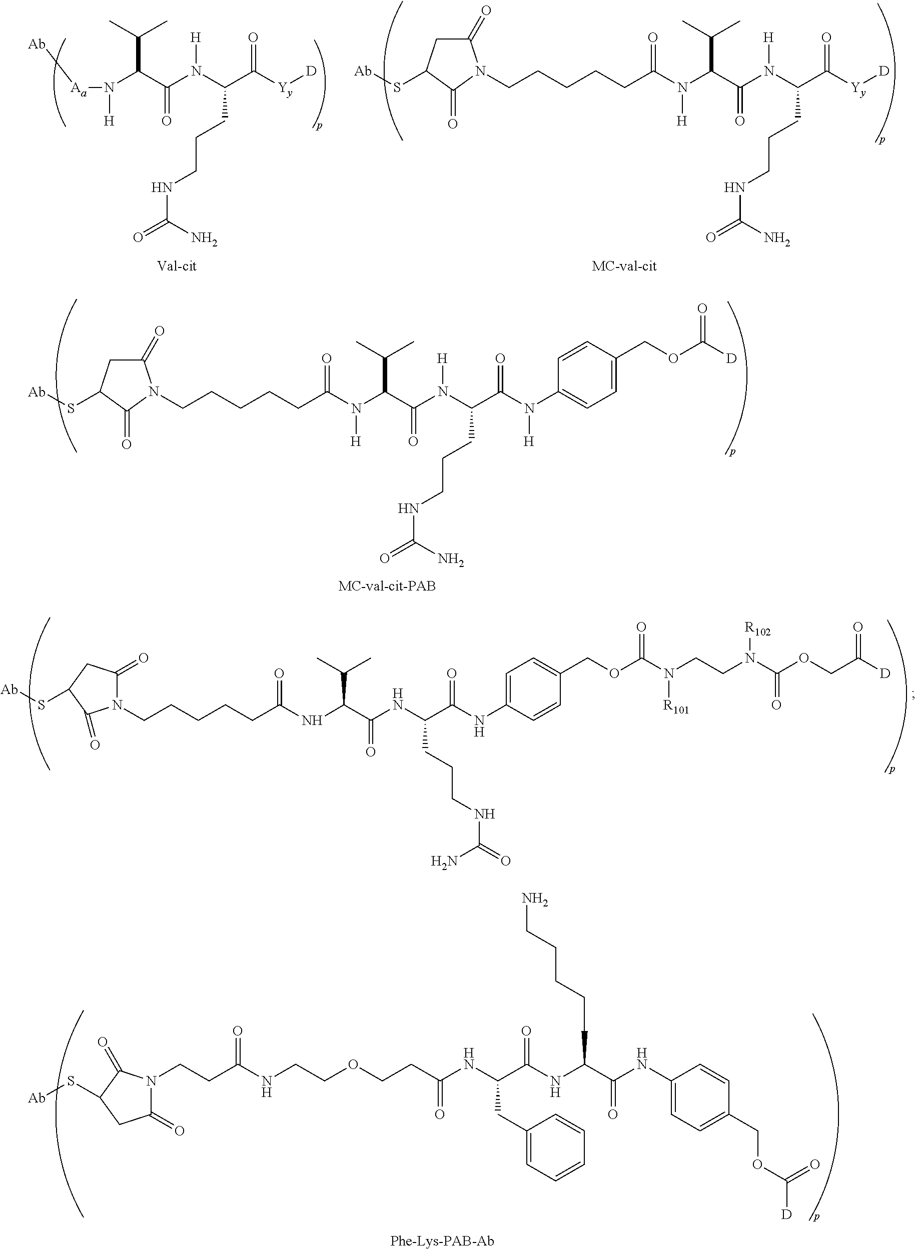

wherein R₁₀₁ and R₁₀₂ are independently selected from H and C₁-C₆ alkyl;
n₅ is an integer from 0 to 12.

In some embodiments, n is an integer 2 to 10. In some embodiments, n is an integer from 4 to 8.

In some embodiments, R₁₀₁ and R₁₀₂ are each —CH₃.

Further nonlimiting exemplary ADCs include the structures:

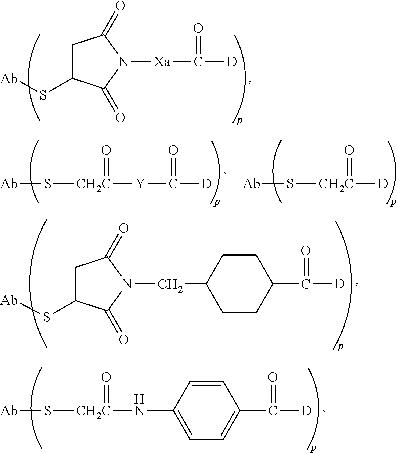

wherein Xₐ is:

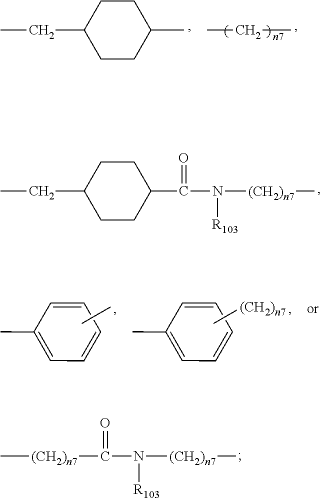

### Y is:

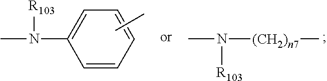

each R₁₀₃ is independently H or C₁-C₆ alkyl; and n7 is an integer from 1 to 12.

In some embodiments, a linker is substituted with groups that modulate solubility and/or reactivity. As a nonlimiting example, a charged substituent such as sulfonate (—SO₃⁻) or ammonium may increase water solubility of the linker reagent and facilitate the coupling reaction of the linker reagent with the antibody and/or the drug moiety, or facilitate the coupling reaction of Ab-L (antibody-linker intermediate) with D, or D-L (drug-linker intermediate) with Ab, depending on the synthetic route employed to prepare the ADC. In some embodiments, a portion of the linker is coupled to the antibody and a portion of the linker is coupled to the drug, and then the Ab-(linker portion)^{a} is coupled to drug-(linker portion)^{b} to form the ADC of Formula Ig.

The compounds disclosed herein expressly contemplate, but are not limited to, ADC prepared with the following linker reagents: bis-maleimido-trioxyethylene glycol (BMPEO), N-(β-maleimidopropyloxy)-N-hydroxy succinimide ester (BMPS), N-(ε-maleimidocaproyloxy) succinimide ester (EMCS), N-[y-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(β-maleimidopropionamido)hexanoate](SMPH), iminothiolane (IT), sulfo-EMCS, sulfo-GMBS, sulfo-KMUS, sulfo-MBS, sulfo-SIAB, sulfo-SMCC, and sulfo-SMPB, and succinimidyl-(4-vinylsulfone)benzoate (SVSB), and including bis-maleimide reagents: dithiobismaleimidoethane (DTME), 1,4-Bismaleimidobutane (BMB), 1,4 Bismaleimidyl-2,3-dihydroxybutane (BMDB), bismaleimidohexane (BMH), bismaleimidoethane (BMOE), BM(PEG)₂ (shown below), and BM(PEG)₃ (shown below); bifunctional derivatives of imidoesters (such as dimethyl adipimidate HCl), active esters (such as disuccinimidyl suberate), aldehydes (such as glutaraldehyde), bis-azido compounds (such as bis-(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In some embodiments, bis-maleimide reagents allow the attachment of the thiol group of a cysteine in the antibody to a thiol-containing drug moiety, linker, or linker-drug intermediate. Other functional groups that are reactive with thiol groups include, but are not limited to, iodoacetamide, bromoacetamide, vinyl pyridine, disulfide, pyridyl disulfide, isocyanate, and isothiocyanate.

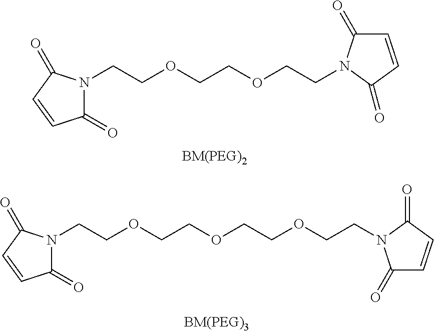 [SMILES: O=C1C=CC(=O)N1CCOCCOCCN1C(=O)C=CC1=O ; O=C1C=CC(=O)N1CCOCCOCCOCCN1C(=O)C=CC1=O]

Certain useful linker reagents can be obtained from various commercial sources, such as Pierce Biotechnology, Inc. (Rockford, Ill.), Molecular Biosciences Inc. (Boulder, Colo.), or synthesized in accordance with procedures described in the art; for example, in Toki et al (2002) J. Org. Chem. 67:1866-1872; Dubowchik, et al. (1997) Tetrahedron Letters, 38:5257-60; Walker, M. A. (1995) J. Org. Chem. 60:5352-5355; Frisch et al (1996) Bioconjugate Chem. 7:180-186; U.S. Pat. No. 6,214,345; WO 02/088172; US 2003130189; US2003096743; WO 03/026577; WO 03/043583; and WO 04/032828.

Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO 94/11026.

### Methods of Making HER2 Antibody Conjugates

In certain embodiments, the conjugates are formed in several steps. These steps include (1) modifying a polymer so that it contains a functional group that can react with a functional group of the drug or its derivative; (2) reacting the modified polymer with the drug or its derivative so that the drug is linked to the polymer; (3) modifying the polymer-drug conjugate so that the polymer contains a functional group that can react with a functional group of the antibody or antigen-binding fragment thereof or its derivative; and (4) reacting the modified polymer-drug conjugate with the antibody or antigen-binding fragment thereof to form the conjugate disclosed herein. Step (3) may be omitted if the modified polymer produced by step (1) contains a functional group that can react with a functional group of the antibody or antigen-binding fragment thereof.

In another embodiment the conjugates are formed in several steps: (1) modifying a polymer so that it contains a functional group that can react with a functional group of a first drug or its derivative; (2) reacting the modified polymer with the first drug or its derivative so that the first drug is linked to the polymer; (3) modifying the polymer-drug conjugate so that it contains a different functional group that can react with a functional group of a second drug or its derivative (4) reacting the modified polymer-drug conjugate with the second drug or its derivative so that the second drug is linked to the polymer-drug conjugate; (5) modifying the polymer-drug conjugate containing 2 different drugs so that the polymer contains a functional group that can react with a functional group of the antibody or antigen-binding fragment thereof, and (6) reacting the modified polymer-drug conjugate of step (5) with the antibody or antigen-binding fragment thereof or its derivative to form the conjugate disclosed herein. Steps (5) and (6) may be repeated if 2 different antibodies or antigen-binding fragments thereof or their derivatives are to be conjugated to form a polymer-drug conjugate comprising two different drugs and two different antibodies or antigen-binding fragments thereof.

In yet another embodiment, the conjugates are formed in several steps. These steps include (1) modifying a polymer so that it contains a functional group that can react with a functional group of the drug or its derivative; (2) further modifying the polymer so that it also contains a functional group that can react with a functional group of the antibody or antigen-binding fragment thereof; (3) reacting the modified polymer with the drug or its derivative so that the drug is linked to the polymer; and (4) reacting the modified polymer-drug conjugate with the antibody or antigen-binding fragment thereof to form the conjugate disclosed herein. The sequence of steps (1) and (2) or that of steps (3) and (4) can be reversed. Further either step (1) or (2) may be omitted if the modified polymer contains a functional group that can react with both a functional group of the drug or its derivatives and a functional group of the antibody or antigen-binding fragment thereof.

In another embodiment the conjugates are formed in several steps: (1) modifying a polymer so that it contains a functional group that can react with a functional group of a first drug or its derivative; (2) further modifying a polymer so that it contains a functional group that can react with a functional group of the antibody or antigen-binding fragment thereof; (3) reacting the modified polymer with the first drug or its derivative so that the first drug is linked to the polymer; (4) modifying the polymer-drug conjugate so that it contains a different functional group that can react with a functional group of a second drug or its derivative (5) reacting the modified polymer-drug conjugate with the second drug or its derivative so that the second drug is linked to the polymer-drug conjugate; (6) reacting the modified polymer-drug conjugate containing 2 different drugs so that the polymer with the antibody or antigen-binding fragment thereof or its derivative to form the conjugate disclosed herein. Step (6) may be repeated if 2 different antibodies or antigen-binding fragments thereof or their derivatives are to be conjugated to form a polymer-drug conjugate comprising two different drugs and two different antibodies or antigen-binding fragment thereof. Step (4) may be carried out after step (1) so that the modified polymer contains two different functional groups that can react with two different drugs or their derivatives. In this embodiment, the modified polymer containing two different functional group that can react with two different drugs or their derivatives can be further modified so that it contains a functional group that can react with a functional group of the antibody or antigen-binding fragment thereof; prior to the reaction of the modified polymer with either the two different drugs (step (3) and step (5) or antibody or antigen-binding fragment thereof (step (6).

In certain exemplary embodiments, the conjugates disclosed herein find use in biomedical applications, such as drug delivery and tissue engineering, and the polymeric carrier is biocompatible and biodegradable. In certain embodiments, the carrier is a soluble polymer, nanoparticle, gel, liposome, micelle, suture, implant, etc. In certain embodiments, the term “soluble polymer” encompasses biodegradable biocompatible polymer such as a polyal (e.g., hydrophilic polyacetal or polyketal). In certain other embodiments, the carrier is a fully synthetic, semi-synthetic or naturally-occurring polymer. In certain other embodiments, the carrier is hydrophilic. Examples of suitable polymeric carrier for producing the conjugates disclosed herein are described in U.S. Pat. No. 8,815,226, the content of which is hereby incorporated by reference in its entirety.

In one embodiment, the polymeric carrier comprises units of Formula (IV):

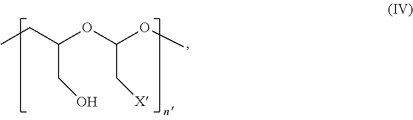

wherein X′ indicates the substituent for the hydroxyl group of the polymer backbone. As shown in Formula (IV) and the other formulae described herein, each polyacetal unit has a single hydroxyl group attached to the glycerol moiety of the unit and an X′ group attached to the glycolaldehyde moiety of the unit. This is for convenience only and it should be construed that the polymer having units of Formula (IV) and other formulae described herein can contain a random distribution of units having a X′ group (or another substituent such as a linker comprising a maleimide terminus) attached to the glycolaldehyde moiety of the units and those having a single X′ group (or another substituent such as a linker comprising a maleimide terminus) attached to the glycerol moiety of the units as well as units having two X′ groups (or other substituents such as a linker comprising a maleimide terminus) with one attached to the glycolaldehyde moiety and the other attached to the glycerol moiety of the units.

In one embodiment, biodegradable biocompatible polyals suitable for practicing the present invention have a molecular weight of between about 0.5 and about 300 kDa. For example, the biodegradable biocompatible polyals have a molecular weight of between about 1 and about 300 kDa (e.g., between about 1 and about 200 kDa, between about 2 and about 300 kDa, between about 2 and about 200 kDa, between about 5 and about 100 kDa, between about 10 and about 70 kDa, between about 20 and about 50 kDa, between about 20 and about 300 kDa, between about 40 and about 150 kDa, between about 50 and about 100 kDa, between about 2 and about 40 kDa, between about 6 and about 20 kDa, or between about 8 and about 15 kDa). For example, the biodegradable biocompatible polyal used for the polymer scaffold or conjugate disclosed herein is PHF having a molecular weight of between about 2 and about 40 kDa (e.g., about 2-20 kDa, 3-15 kDa, or 5-10 kDa.)

Methods for preparing polymer carriers (e.g., biocompatible, biodegradable polymer carriers) suitable for conjugation to modifiers are known in the art. For example, synthetic guidance can be found in U.S. Pat. Nos. 5,811,510; 5,863,990; 5,958,398; 7,838,619; 7,790,150; and 8,685,383. The skilled practitioner will know how to adapt these methods to make polymer carriers for use in the practice of the invention.

In one embodiment, a method for forming the biodegradable biocompatible polyal conjugates of the present disclosure comprises a process by which a suitable polysaccharide is combined with an efficient amount of a glycol-specific oxidizing agent to form an aldehyde intermediate. The aldehyde intermediate, which is a polyal itself, may then be reduced to the corresponding polyol, succinylated, and coupled with one or more suitable modifiers to form a biodegradable biocompatible polyal conjugate comprising succinamide-containing linkages.

In another preferred embodiment, fully synthetic biodegradable biocompatible polyals for used in the present invention can be prepared by reacting a suitable initiator with a suitable precursor compound.

For example, fully synthetic polyals may be prepared by condensation of vinyl ethers with protected substituted diols. Other methods, such as cycle opening polymerization, may be used, in which the method efficacy may depend on the degree of substitution and bulkiness of the protective groups.

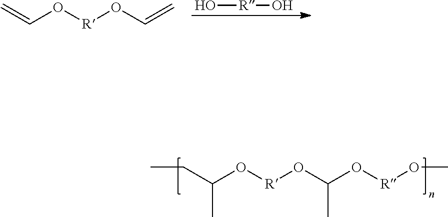

One of ordinary skill in the art will appreciate that solvent systems, catalysts and other factors may be optimized to obtain high molecular weight products.

In certain embodiments, the carrier is PHF.

In embodiments, the polymer carrier is PHF having a polydispersity index (PDI) of ≤1.5, e.g., <1.4, <1.3, <1.2 or <1.1.

For example, for conjugating the antibody or antigen-binding fragment thereof having a molecular weight of 40 kDa to 200 kDa, the polymeric carrier of the scaffold is a polyacetal, e.g., a PHF having a molecular weight (i.e., MW of the unmodified PHF) ranging from about 2 kDa to about 40 kDa (e.g., about 2-20 kDa, or about 3-15 kDa, or about 5-10 kDa).

For example, for conjugating the antibody or antigen-binding fragment thereof having a molecular weight of 40 kDa to 80 kDa, the polymeric carrier of the scaffold disclosed herein is a polyacetal, e.g., a PHF having a molecular weight (i.e., MW of the unmodified PHF) ranging from about 2 kDa to about 40 kDa (e.g., about 2-20 kDa, or about 3-15 kDa, or about 5-10 kDa). For example the PHF has a molecular weight of about 5 kDa, 6 kDa, 7 kDa, 8 kDa, 9 kDa, 10 kDa, 11 kDa, 12 kDa, 13 kDa, 14 kDa, or 15 kDa.

The antibody or antigen-binding fragment thereof in this molecular weight range, includes but are not limited to, for example, antibody fragments, such as, for example, Fabs.

For example, for conjugating the antibody or antigen-binding fragment thereof having a molecular weight of 60 kDa to 120 kDa, the polymeric carrier of the scaffold disclosed herein is a polyacetal, e.g., a PHF having a molecular weight (i.e., MW of the unmodified PHF) ranging from about 2 kDa to about 40 kDa (e.g., about 2-20 kDa, or about 3-15 kDa, or about 5-10 kDa). For example the PHF has a molecular weight of about 5 kDa, 6 kDa, 7 kDa, 8 kDa, 9 kDa, 10 kDa, 11 kDa, 12 kDa, 13 kDa, 14 kDa, or 15 kDa.

The antibody or antigen-binding fragment thereof in this molecular weight range, includes but are not limited to, for example, camelids, Fab2, scFvFc, and the like.

For example, for conjugating the antibody or antigen-binding fragment thereof having a molecular weight of 140 kDa to 180 kDa or of 140 kDa to 150 kDa, the polymeric carrier of the scaffold disclosed herein is a polyacetal, e.g., a PHF having a molecular weight (i.e., MW of the unmodified PHF) ranging from about 2 kDa to about 40 kDa (e.g., about 2-20 kDa, or about 3-15 kDa, or about 5-10 kDa). For example the PHF has a molecular weight of about 5 kDa, 6 kDa, 7 kDa, 8 kDa, 9 kDa, 10 kDa, 11 kDa, 12 kDa, 13 kDa, 14 kDa, or 15 kDa.

The antibody or antigen-binding fragment thereof in this molecular weight range, includes but are not limited to, for example, full length antibodies, such as, IgG, IgM.

The biodegradable biocompatible conjugates disclosed herein can be prepared to meet desired requirements of biodegradability and hydrophilicity. For example, under physiological conditions, a balance between biodegradability and stability can be reached. For instance, it is known that molecules with molecular weights beyond a certain threshold (generally, above 40-100 kDa, depending on the physical shape of the molecule) are not excreted through kidneys, as small molecules are, and can be cleared from the body only through uptake by cells and degradation in intracellular compartments, most notably lysosomes. This observation exemplifies how functionally stable yet biodegradable materials may be designed by modulating their stability under general physiological conditions (pH=7.5+0.5) and at lysosomal pH (pH near 5). For example, hydrolysis of acetal and ketal groups is known to be catalyzed by acids, therefore polyals will be in general less stable in acidic lysosomal environment than, for example, in blood plasma. One can design a test to compare polymer degradation profile at, for example, pH=5 and pH=7.5 at 37° C. in aqueous media, and thus to determine the expected balance of polymer stability in normal physiological environment and in the “digestive” lysosomal compartment after uptake by cells. Polymer integrity in such tests can be measured, for example, by size exclusion HPLC. One skilled on the art can select other suitable methods for studying various fragments of the degraded conjugates disclosed herein.

In many cases, it will be preferable that at pH=7.5 the effective size of the polymer will not detectably change over 1 to 7 days, and remain within 50% from the original for at least several weeks. At pH=5, on the other hand, the polymer should preferably detectably degrade over 1 to 5 days, and be completely transformed into low molecular weight fragments within a two-week to several-month time frame. Although faster degradation may be in some cases preferable, in general it may be more desirable that the polymer degrades in cells with the rate that does not exceed the rate of metabolization or excretion of polymer fragments by the cells. Accordingly, in certain embodiments, the conjugates of the present disclosure are expected to be biodegradable, in particular upon uptake by cells, and relatively “inert” in relation to biological systems. The products of carrier degradation are preferably uncharged and do not significantly shift the pH of the environment. It is proposed that the abundance of alcohol groups may provide low rate of polymer recognition by cell receptors, particularly of phagocytes. The polymer backbones of the present disclosure generally contain few, if any, antigenic determinants (characteristic, for example, for some polysaccharides and polypeptides) and generally do not comprise rigid structures capable of engaging in “key-and-lock” type interactions in vivo unless the latter are desirable. Thus, the soluble, crosslinked and solid conjugates disclosed herein are predicted to have low toxicity and bioadhesivity, which makes them suitable for several biomedical applications.

In certain embodiments of the present invention, the biodegradable biocompatible conjugates can form linear or branched structures. For example, the biodegradable biocompatible polyal conjugates of the present disclosure can be chiral (optically active). Optionally, the biodegradable biocompatible polyal conjugates of the present disclosure can be scalemic.

In certain embodiments, the conjugates disclosed herein are water-soluble. In certain embodiments, the conjugates disclosed herein are water-insoluble. In certain embodiments, the inventive conjugate is in a solid form. In certain embodiments, the conjugates disclosed herein are colloids. In certain embodiments, the conjugates disclosed herein are in particle form. In certain embodiments, the conjugates disclosed herein are in gel form.

Methods for preparing the polymeric scaffolds and conjugates disclosed herein can also be found in U.S. Application Nos. 62/523,378, 62/545,296, and 62/623,275, the entire contents of each of which are hereby incorporated by reference in their entireties.

Scheme 1 below shows a synthetic scheme of making a polymeric drug scaffold disclosed herein. In one embodiment, the conjugates are formed in several steps: (1) the polymer, PHF is modified to contain a COOH moiety (e.g., —C(O)—X—(CH₂)₂—COOH); (2) the polymer is then further modified so that it contains a maleimido moiety (e.g., EG2-MI) that can react with a functional group of a PBRM; (3) the modified polymer, containing two different functional groups, is reacted with a functional group of a drug or its derivative (e.g., AF-HPA-Ala) to form a polymer-drug conjugate; (4) the disulfide bonds of a PBRM are reduced; (5) the reduced PBRM is then reacted with the polymer-drug conjugate to form the protein-polymer-drug conjugate; and (6) the remaining maleimido moieties are optionally reacted with a maleimido blocking compound (e.g., cysteine).

In another embodiment the order of steps (2) and (3) can be reversed as depicted in the right side route in Scheme 1 below.

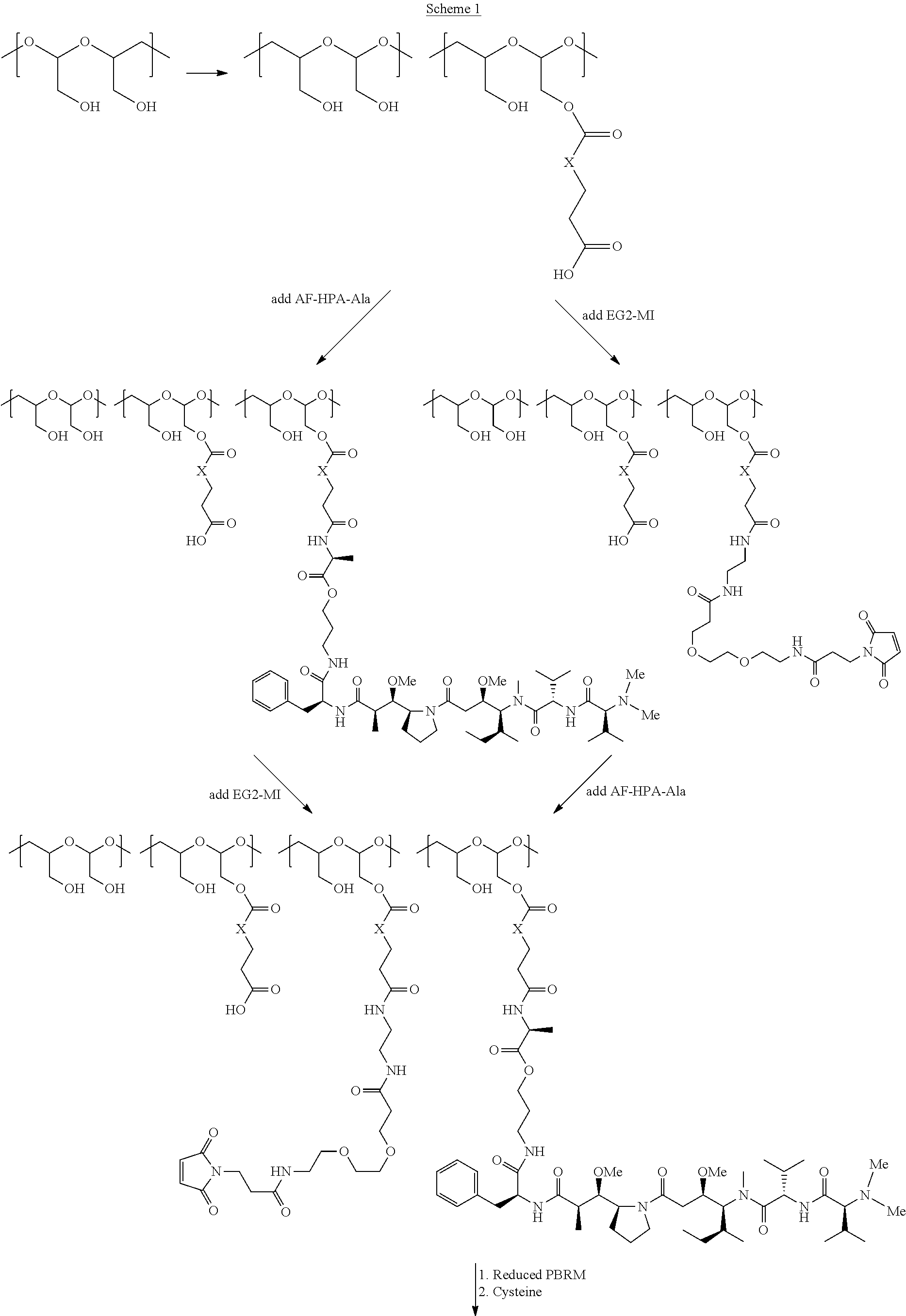

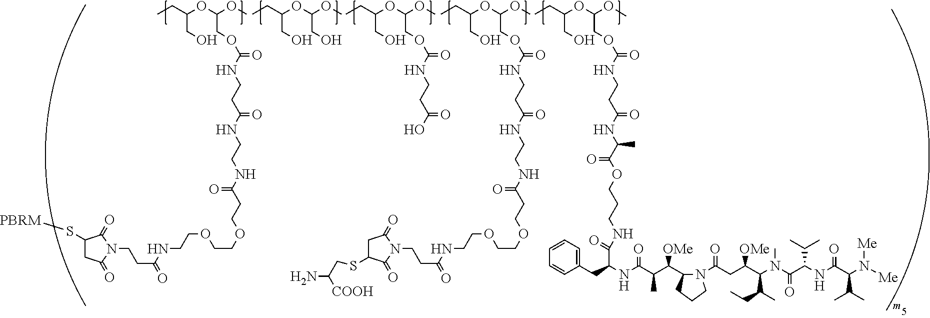

In yet another embodiment, steps (2) and (3) above are carried out simultaneously as depicted in Scheme 2 below.

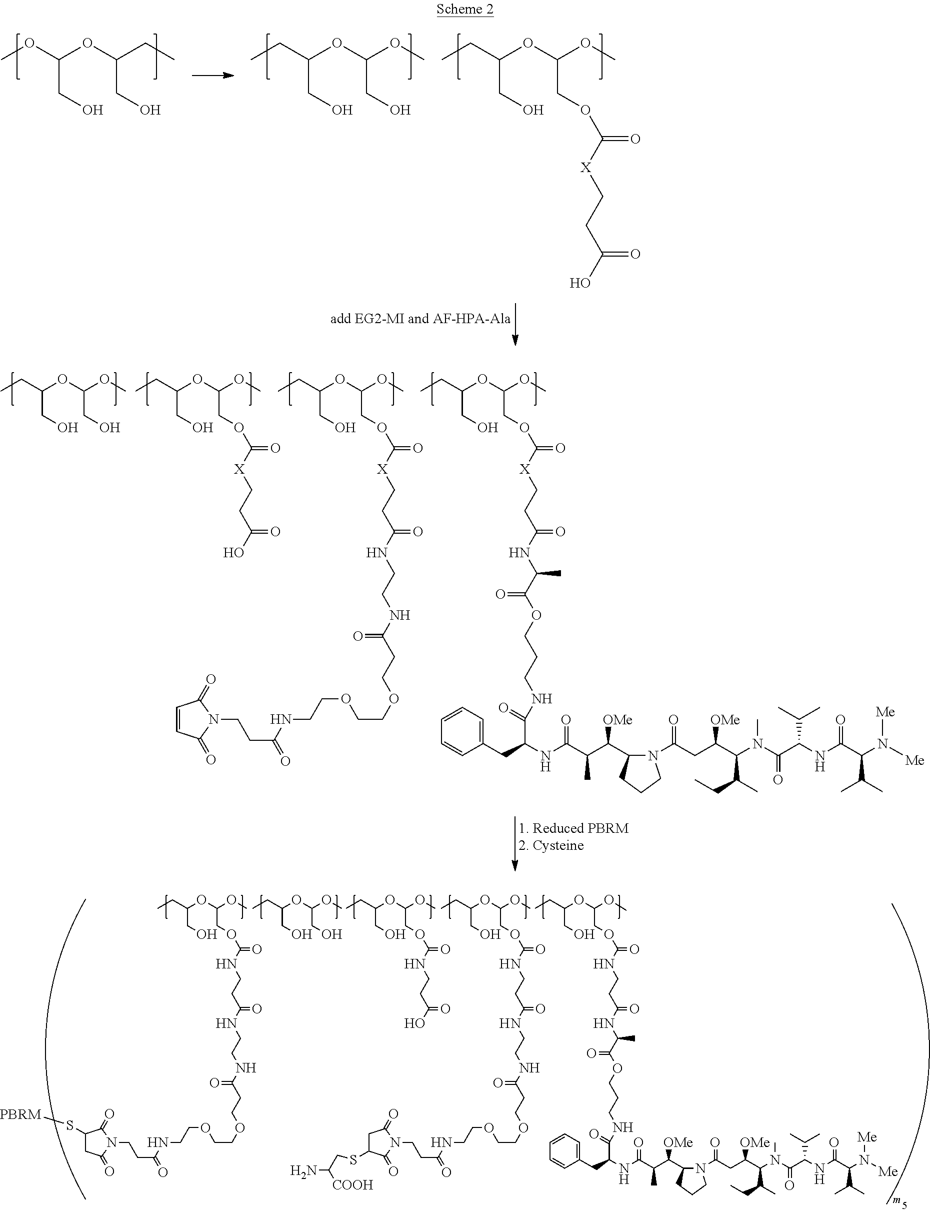

### Immune Checkpoint Inhibitors

Any suitable immune checkpoint inhibitors have been contemplated herein for use in the combinations and methods of the disclosure. Immune checkpoint inhibitors can include, but are not limited to, immune checkpoint molecule binding proteins, small molecule inhibitors, antibodies, antibody-derivatives (including Fab fragments and scFvs), antibody-drug conjugates, antisense oligonucleotides, siRNA, aptamers, peptides and peptide mimetics. Inhibitory nucleic acids that decrease the expression and/or activity of immune checkpoint molecules can also be used in the combinations and methods disclosed herein.

In one embodiment, the immune checkpoint inhibitor reduces the expression or activity of one or more immune checkpoint proteins. In another embodiment, the immune checkpoint inhibitor reduces the interaction between one or more immune checkpoint proteins and their ligands. See, e.g., US20160101128.

In some embodiments, the immune checkpoint inhibitor is an inhibitor of CTLA-4. In some embodiments, the immune checkpoint inhibitor is an antibody against CTLA-4. In some embodiments, the immune checkpoint inhibitor is a monoclonal antibody against CTLA-4. In other embodiments, the immune checkpoint inhibitor is a human or humanized antibody against CTLA-4. In one embodiment, the anti-CTLA-4 antibody blocks the binding of CTLA-4 to CD80 (B7-1) and/or CD86 (B7-2) expressed on antigen presenting cells. Exemplary antibodies against CTLA-4 include, but are not limited to, Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); and anti-CTLA4 antibody clone BNI3 from Abcam.

In some embodiments, the anti-CTLA-4 antibody is an anti-CTLA-4 antibody disclosed in any of the following patent publications (herein incorporated by reference): WO 2001014424; WO 2004035607; US2005/0201994; EP 1212422 B1; WO2003086459; WO2012120125; WO2000037504; WO2009100140; W0200609649; WO2005092380; WO2007123737; WO2006029219; WO20100979597; W0200612168; and WO1997020574.

Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014; and/or U.S. Pat. Nos. 5,977,318, 6,682,736, 7,109,003, and 7,132,281, incorporated herein by reference). In some embodiments, the anti-CTLA-4 antibody is for example, those disclosed in: WO 98/42752; U.S. Pat. Nos. 6,682,736 and 6,207,156; Hurwitz et al, Proc. Natl. Acad. Sci. USA, 95(17): 10067-10071 (1998); Camacho et al, J. Clin. Oncol., 22(145): Abstract No. 2505 (2004) (antibody CP-675206); Mokyr et al, Cancer Res., 58:5301-5304 (1998) (incorporated herein by reference).

In some embodiments, the CTLA-4 inhibitor is a CTLA-4 ligand as disclosed in WO1996040915.

In some embodiments, the CTLA-4 inhibitor is a nucleic acid inhibitor of CTLA-4 expression. For example, anti-CTLA4 RNAi molecules may take the form of the molecules described by Mello and Fire in PCT Publication Nos. WO 1999/032619 and WO 2001/029058; U.S. Publication Nos. 2003/0051263, 2003/0055020, 2003/0056235, 2004/265839, 2005/0100913, 2006/0024798, 2008/0050342, 2008/0081373, 2008/0248576, and 2008/055443; and/or U.S. Pat. Nos. 6,506,559, 7,282,564, 7,538,095, and 7,560,438 (incorporated herein by reference). In some instances, the anti-CTLA4 RNAi molecules take the form of double stranded RNAi molecules described by Tuschl in European Patent No. EP 1309726 (incorporated herein by reference). In some instances, the anti-CTLA4 RNAi molecules take the form of double stranded RNAi molecules described by Tuschl in U.S. Pat. Nos. 7,056,704 and 7,078,196 (incorporated herein by reference). In some embodiments, the CTLA4 inhibitor is an aptamer described in PCT Publication No. WO2004081021.

Additionally, the anti-CTLA4 RNAi molecules of the present disclosure may take the form be RNA molecules described by Crooke in U.S. Pat. Nos. 5,898,031, 6,107,094, 7,432,249, and 7,432,250, and European Application No. EP 0928290 (incorporated herein by reference).

In some embodiments, the immune checkpoint inhibitor is an inhibitor of PD-L1. In some embodiments, the immune checkpoint inhibitor is an antibody against PD-L1. In some embodiments, the immune checkpoint inhibitor is a monoclonal antibody against PD-L1. In other or additional embodiments, the immune checkpoint inhibitor is a human or humanized antibody against PD-L1. In one embodiment, the immune checkpoint inhibitor reduces the expression or activity of one or more immune checkpoint proteins, such as PD-L1. In another embodiment, the immune checkpoint inhibitor reduces the interaction between PD-1 and PD-L1. Exemplary immune checkpoint inhibitors include antibodies (e.g., an anti-PD-L1 antibody), RNAi molecules (e.g., anti-PD-L1 RNAi), antisense molecules (e.g., an anti-PD-L1 antisense RNA), dominant negative proteins (e.g., a dominant negative PD-L1 protein), and small molecule inhibitors. Antibodies include monoclonal antibodies, humanized antibodies, deimmunized antibodies, and Ig fusion proteins. An exemplary anti-PD-L1 antibody includes clone EH12. Exemplary antibodies against PD-L1 include: Genentech's MPDL3280A (RG7446); Anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; and AstraZeneca's MEDI4736. In some embodiments, the anti-PD-L1 antibody is an anti-PD-L1 antibody disclosed in any of the following patent publications (herein incorporated by reference): WO2013079174; CN101104640; WO2010036959; WO2013056716; WO2007005874; WO2010089411; WO2010077634; WO2004004771; WO2006133396; W0201309906; US 20140294898; WO2013181634 or WO2012145493.

In some embodiments, the PD-L1 inhibitor is a nucleic acid inhibitor of PD-L1 expression. In some embodiments, the PD-L1 inhibitor is disclosed in one of the following patent publications (incorporated herein by reference): WO2011127180 or WO2011000841. In some embodiments, the PD-L1 inhibitor is rapamycin.

In some embodiments, the immune checkpoint inhibitor is an inhibitor of PD-L2. In some embodiments, the immune checkpoint inhibitor is an antibody against PD-L2. In some embodiments, the immune checkpoint inhibitor is a monoclonal antibody against PD-L2. In other or additional embodiments, the immune checkpoint inhibitor is a human or humanized antibody against PD-L2. In some embodiments, the immune checkpoint inhibitor reduces the expression or activity of one or more immune checkpoint proteins, such as PD-L2. In other embodiments, the immune checkpoint inhibitor reduces the interaction between PD-1 and PD-L2. Exemplary immune checkpoint inhibitors include antibodies (e.g., an anti-PD-L2 antibody), RNAi molecules (e.g., an anti-PD-L2 RNAi), antisense molecules (e.g., an anti-PD-L2 antisense RNA), dominant negative proteins (e.g., a dominant negative PD-L2 protein), and small molecule inhibitors. Antibodies include monoclonal antibodies, humanized antibodies, deimmunized antibodies, and Ig fusion proteins.

In some embodiments, the PD-L2 inhibitor is GlaxoSmithKline's AMP-224 (Amplimmune). In some embodiments, the PD-L2 inhibitor is rHIgM12B7.

In some embodiments, the immune checkpoint inhibitor is an inhibitor of PD-L1. In some embodiments, the immune checkpoint inhibitor is an antibody against PD-1. In some embodiments, the immune checkpoint inhibitor is a monoclonal antibody against PD-1. In other embodiments, the immune checkpoint inhibitor is a human or humanized antibody against PD-1. For example, the inhibitors of PD-1 biological activity (or its ligands) disclosed in U.S. Pat. Nos. 7,029,674; 6,808,710; or U.S. Patent Application Nos: 20050250106 and 20050159351 can be used in the combinations provided herein. Exemplary antibodies against PD-1 include: Anti-mouse PD-1 antibody Clone J43 (Cat #BE0033-2) from BioXcell; Anti-mouse PD-1 antibody Clone RMPi-14 (Cat #BE0146) from BioXcell; mouse anti-PD-1 antibody Clone EH12; Merck's MK-3475 anti-mouse PD-1 antibody (Keytruda®, pembrolizumab, lambrolizumab, h409A1 1); and AnaptysBio's anti-PD-1 antibody, known as ANB011; antibody MDX-1 106 (ONO-4538); Bristol-Myers Squibb's human IgG4 monoclonal antibody nivolumab (Opdivo®, BMS-936558, MDX1106); AstraZeneca's AMP-514, and AMP-224; and Pidilizumab (CT-011 or hBAT-1), CureTech Ltd.

Additional exemplary anti-PD-1 antibodies are described by Goldberg et al, Blood 1 10(1): 186-192 (2007), Thompson et al, Clin. Cancer Res. 13(6): 1757-1761 (2007), and Korman et al, International Application No. PCT/JP2006/309606 (publication no. WO 2006/121168 A1), each of which are expressly incorporated by reference herein. In some embodiments, the anti-PD-1 antibody is an anti-PD-1 antibody disclosed in any of the following patent publications (herein incorporated by reference): W0014557; WO2011110604; WO2008156712; US2012023752; WO2011110621; WO2004072286; WO2004056875; WO20100036959; WO2010029434; W0201213548; WO2002078731; WO2012145493; WO2010089411; WO2001014557; WO2013022091; WO2013019906; WO2003011911; US20140294898; and WO2010001617.

In some embodiments, the PD-1 inhibitor is a PD-1 binding protein as disclosed in W0200914335 (herein incorporated by reference).

In some embodiments, the PD-1 inhibitor is a peptidomimetic inhibitor of PD-1 as disclosed in WO2013132317 (herein incorporated by reference).

In some embodiments, the PD-1 inhibitor is an anti-mouse PD-1 mAb: clone J43, BioXCell (West Lebanon, N.H.).

In some embodiments, the PD-1 inhibitor is a PD-L1 protein, a PD-L2 protein, or fragments, as well as antibody MDX-1 106 (ONO-4538) tested in clinical studies for the treatment of certain malignancies (Brahmer et al., J Clin Oncol. 2010 28(19): 3167-75, Epub 2010 Jun. 1). Other blocking antibodies may be readily identified and prepared by the skilled person based on the known domain of interaction between PD-1 and PD-L1/PD-L2, as discussed above. For example, a peptide corresponding to the IgV region of PD-1 or PD-L1/PD-L2 (or to a portion of this region) could be used as an antigen to develop blocking antibodies using methods well known in the art.

In some embodiments, the immune checkpoint inhibitor is an inhibitor of IDO1. In some embodiments, the immune checkpoint inhibitor is a small molecule against IDO1. Exemplary small molecules against IDO1 include: Incyte's INCB024360, NSC-721782 (also known as 1-methyl-D-tryptophan), and Bristol Meyers Squibb's F001287.

In some embodiments, the immune checkpoint inhibitor is an inhibitor of LAG3 (CD223). In some embodiments, the immune checkpoint inhibitor is an antibody against LAG3. In some embodiments, the immune checkpoint inhibitor is a monoclonal antibody against LAG3. In other or additional embodiments, the immune checkpoint inhibitor is a human or humanized antibody against LAG3. In additional embodiments, an antibody against LAG3 blocks the interaction of LAG3 with major histocompatibility complex (MHC) class II molecules. Exemplary antibodies against LAG3 include: anti-Lag-3 antibody clone eBioC9B7W (C₉B7W) from eBioscience; anti-Lag3 antibody LS-B2237 from LifeSpan Biosciences; IMP321 (ImmuFact) from Immutep; anti-Lag3 antibody BMS-986016; and the LAG-3 chimeric antibody A9H12. In some embodiments, the anti-LAG3 antibody is an anti-LAG3 antibody disclosed in any of the following patent publications (herein incorporated by reference): WO2010019570; WO2008132601; or WO2004078928.

In some embodiments, the immune checkpoint inhibitor is an antibody against TIM3 (also known as HAVCR2). In some embodiments, the immune checkpoint inhibitor is a monoclonal antibody against TIM3. In other or additional embodiments, the immune checkpoint inhibitor is a human or humanized antibody against TIM3. In additional embodiments, an antibody against TIM3 blocks the interaction of TIM3 with galectin-9 (Gal9). In some embodiments, the anti-TIM3 antibody is an anti-TIM3 antibody disclosed in any of the following patent publications (herein incorporated by reference): WO2013006490; W0201155607; WO2011159877; or W0200117057. In another embodiment, a TIM3 inhibitor is a TIM3 inhibitor disclosed in WO2009052623.

In some embodiments, the immune checkpoint inhibitor is an antibody against B7-H3. In one embodiment, the immune checkpoint inhibitor is MGA271.

In some embodiments, the immune checkpoint inhibitor is an antibody against MR. In one embodiment, the immune checkpoint inhibitor is Lirilumab (IPH2101). In some embodiments, an antibody against MR blocks the interaction of KIR with HLA.

In some embodiments, the immune checkpoint inhibitor is an antibody against CD137 (also known as 4-1BB or TNFRSF9). In one embodiment, the immune checkpoint inhibitor is urelumab (BMS-663513, Bristol-Myers Squibb), PF-05082566 (anti-4-1BB, PF-2566, Pfizer), or XmAb-5592 (Xencor). In one embodiment, an anti-CD137 antibody is an antibody disclosed in U.S. Published Application No. US 2005/0095244; an antibody disclosed in issued U.S. Pat. No. 7,288,638 (such as 20H4.9-IgG4 [1007 or BMS-663513] or 20H4.9-IgG1 [BMS-663031]); an antibody disclosed in issued U.S. Pat. No. 6,887,673 [4E9 or BMS-554271]; an antibody disclosed in issued U.S. Pat. No. 7,214,493; an antibody disclosed in issued U.S. Pat. No. 6,303,121; an antibody disclosed in issued U.S. Pat. No. 6,569,997; an antibody disclosed in issued U.S. Pat. No. 6,905,685; an antibody disclosed in issued U.S. Pat. No. 6,355,476; an antibody disclosed in issued U.S. Pat. No. 6,362,325 [1D8 or BMS-469492; 3H3 or BMS-469497; or 3E1]; an antibody disclosed in issued U.S. Pat. No. 6,974,863 (such as 53A2); or an antibody disclosed in issued U.S. Pat. No. 6,210,669 (such as 1D8, 3B8, or 3E1). In a further embodiment, the immune checkpoint inhibitor is one disclosed in WO 2014036412. In another embodiment, an antibody against CD137 blocks the interaction of CD137 with CD137L.

In some embodiments, the immune checkpoint inhibitor is an antibody against PS. In one embodiment, the immune checkpoint inhibitor is Bavituximab.

In some embodiments, the immune checkpoint inhibitor is an antibody against CD52. In one embodiment, the immune checkpoint inhibitor is alemtuzumab.

In some embodiments, the immune checkpoint inhibitor is an antibody against CD30. In one embodiment, the immune checkpoint inhibitor is brentuximab vedotin. In another embodiment, an antibody against CD30 blocks the interaction of CD30 with CD30L.

In some embodiments, the immune checkpoint inhibitor is an antibody against CD33. In one embodiment, the immune checkpoint inhibitor is gemtuzumab ozogamicin.

In some embodiments, the immune checkpoint inhibitor is an antibody against CD20. In one embodiment, the immune checkpoint inhibitor is ibritumomab tiuxetan. In another embodiment, the immune checkpoint inhibitor is ofatumumab. In another embodiment, the immune checkpoint inhibitor is rituximab. In another embodiment, the immune checkpoint inhibitor is tositumomab.

In some embodiments, the immune checkpoint inhibitor is an antibody against CD27 (also known as TNFRSF7). In one embodiment, the immune checkpoint inhibitor is CDX-1127 (Celldex Therapeutics). In another embodiment, an antibody against CD27 blocks the interaction of CD27 with CD70.

In some embodiments, the immune checkpoint inhibitor is an antibody against OX40 (also known as TNFRSF4 or CD134). In one embodiment, the immune checkpoint inhibitor is anti-OX40 mouse IgG. In another embodiment, an antibody against 0×40 blocks the interaction of OX40 with OX40L.

In some embodiments, the immune checkpoint inhibitor is an antibody against glucocorticoid-induced tumor necrosis factor receptor (GITR). In one embodiment, the immune checkpoint inhibitor is TRX518 (GITR, Inc.). In another embodiment, an antibody against GITR blocks the interaction of GITR with GITRL.

In some embodiments, the immune checkpoint inhibitor is an antibody against inducible T-cell COStimulator (ICOS, also known as CD278). In one embodiment, the immune checkpoint inhibitor is MEDI570 (MedImmune, LLC) or AMG557 (Amgen). In another embodiment, an antibody against ICOS blocks the interaction of ICOS with ICOSL and/or B7-H2.

In some embodiments, the immune checkpoint inhibitor is an inhibitor against BTLA (CD272), CD160, 2B4, LAIR1, TIGHT, LIGHT, DR3, CD226, CD2, or SLAM. As described elsewhere herein, an immune checkpoint inhibitor can be one or more binding proteins, antibodies (or fragments or variants thereof) that bind to immune checkpoint molecules, nucleic acids that downregulate expression of the immune checkpoint molecules, or any other molecules that bind to immune checkpoint molecules (i.e. small organic molecules, peptidomimetics, aptamers, etc.). In some instances, an inhibitor of BTLA (CD272) is HVEM. In some instances, an inhibitor of CD160 is HVEM. In some cases, an inhibitor of 2B4 is CD48. In some instances, an inhibitor of LAIR1 is collagen. In some instances, an inhibitor of TIGHT is CD112, CD113, or CD155. In some instances, an inhibitor of CD28 is CD80 or CD86. In some instances, an inhibitor of LIGHT is HVEM. In some instances, an inhibitor of DR3 is TL1A. In some instances, an inhibitor of CD226 is CD155 or CD112. In some cases, an inhibitor of CD2 is CD48 or CD58. In some cases, SLAM is self-inhibitory and an inhibitor of SLAM is SLAM.

In certain embodiments, the immune checkpoint inhibitor inhibits a checkpoint protein that include, but are not limited to CTLA4 (cytotoxic T-lymphocyte antigen 4, also known as CD152), PD-L1 (programmed cell death 1 ligand 1, also known as CD274), PDL2 programmed cell death protein 2), PD-1 (programmed cell death protein 1, also known as CD279), a B-7 family ligand (B7-H1, B7-H3, B7-H4) BTLA (B and T lymphocyte attenuator, also known as CD272), HVEM, TIM3 (T-cell membrane protein 3), GAL9, LAG-3 (lymphocyte activation gene-3; CD223), VISTA, KIR (killer immunoglobulin receptor), 2B4 (also known as CD244), CD160, CGEN-15049, CHK1 (Checkpoint kinase 1), CHK2 (Checkpoint kinase 2), A2aR (adenosine A2a receptor), CD2, CD27, CD28, CD30, CD40, CD70, CD80, CD86, CD137, CD226, CD276, DR3, GITR, HAVCR2, HVEM, IDO1 (indoleamine 2,3-dioxygenase 1), IDO2 (indoleamine 2,3-dioxygenase 2), ICOS (inducible T cell costimulator), LAIR1, LIGHT (also known as TNFSF14, a TNF family members, MARCO (macrophage receptor with collagenous structure), OX40 (also known as tumor necrosis factor receptor superfamily, member 4, TNFRSF4, and CD134) and its ligand OX40L (CD252), SLAM, TIGHT, VTCN1 or a combination thereof.

In certain embodiments, the immune checkpoint inhibitor interacts with a ligand of a checkpoint protein that comprises CTLA-4, PDL1, PDL2, PD1, BTLA, HVEM, TIM3, GAL9, LAG3, VISTA, KIR, 2B4, CD160, CGEN-15049, CHK1, CHK2, A2aR, a B-7 family ligand, CD2, CD27, CD28, CD30, CD40, CD70, CD80, CD86, CD137, CD226, CD276, DR3, GITR, HAVCR2, HVEM, IDO1, IDO2, ICOS (inducible T cell costimulator), LAIR1, LIGHT, MARCO (macrophage receptor with collagenous structure), OX-40, SLAM, TIGHT, VTCN1 or a combination thereof.

In certain embodiments, the immune checkpoint inhibitor inhibits a checkpoint protein that comprises CTLA-4, PDL1, PD1 or a combination thereof.

In certain embodiments, the immune checkpoint inhibitor inhibits a checkpoint protein that comprises CTLA-4 and PD1 or a combination thereof.

In certain embodiments, the immune checkpoint inhibitor comprises pembrolizumab (MK-3475), nivolumab (BMS-936558), pidilizumab (CT-011), AMP-224, MDX-1 105, durvalumab (MEDI4736), MPDL3280A, BMS-936559, IPH2101, TSR-042, TSR-022, ipilimumab, lirilumab, atezolizumab, avelumab, tremelimumab, or a combination thereof.

In certain embodiments, the immune checkpoint inhibitor is nivolumab (BMS-936558), ipilimumab, pembrolizumab, atezolizumab, tremelimumab, durvalumab, avelumab, or a combination thereof.

In certain embodiments, the immune checkpoint inhibitor is pembrolizumab.

### Combination Therapies and Formulations of HER2-Targeted Antibody-Drug Conjugates and Immune Checkpoint Inhibitors

It will be appreciated that administration of the conjugates and immune checkpoint inhibitors in combination of the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed., Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman WN “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

In one embodiment, the conjugates and immune checkpoint inhibitors disclosed herein may be used as therapeutic agents. Such agents will generally be employed to diagnose, prognose, monitor, treat, alleviate, prevent, and/or delay the progression of a disease or pathology associated with, e.g., an aberrant HER2 activity and/or expression in a subject. A therapeutic regimen is carried out by identifying a subject, e.g., a human patient suffering from (or at risk of developing) a disease or disorder associated with aberrant HER2 activity and/or expression, e.g., a cancer, using standard methods. Antibody conjugate preparation, preferably one having high specificity and high affinity for its target antigen, is administered to the subject and will generally have an effect due to its binding with the target. Administration of the conjugate may abrogate or inhibit or interfere with the signaling function of the target. Administration of the conjugate may abrogate or inhibit or interfere with the binding of the target with an endogenous ligand to which it naturally binds. For example, the conjugate binds to the target and modulates, blocks, inhibits, reduces, antagonizes, neutralizes, or otherwise interferes with HER2 activity and/or expression. Administration of the conjugate may also exhibit therapeutic effects by targeted delivery of the therapeutic agents attached to the conjugate.

Diseases or disorders related to aberrant HER2 activity and/or expression include but not limited to cancer. The target cancer can be anal, astrocytoma, leukemia, lymphoma, head and neck, liver, testicular, cervical, sarcoma, hemangioma, esophageal, eye, laryngeal, mouth, mesothelioma, skin, myeloma, oral, rectal, throat, bladder, breast, uterus, ovary, prostate, lung, colon, pancreas, renal, or gastric cancer.

In another aspect, diseases or disorders are cancer selected from the group consisting of breast cancer, gastric cancer, non-small cell lung cancer (NSCLC), and ovarian cancer.

Generally, alleviation or treatment of a disease or disorder involves the lessening of one or more symptoms or medical problems associated with the disease or disorder. For example, in the case of cancer, the therapeutically effective amount of the drug can accomplish one or a combination of the following: reduce the number of cancer cells; reduce the tumor size; inhibit (i.e., to decrease to some extent and/or stop) cancer cell infiltration into peripheral organs; inhibit tumor metastasis; inhibit, to some extent, tumor growth; and/or relieve to some extent one or more of the symptoms associated with the cancer. In some embodiments, a composition disclosed herein can be used to prevent the onset or reoccurrence of the disease or disorder in a subject.

An effective or sufficient amount of a combination of conjugate and immune checkpoint inhibitor disclosed herein relate generally to the amount (e.g., the amount of the conjugate and that of the checkpoint inhibitor) needed to achieve a therapeutic objective. As noted above, this may be a binding interaction between the antibody of the conjugate and its target antigen that, in certain cases, interferes with the functioning of the target. The amount required to be administered will furthermore depend on the binding affinity of the antibody of the conjugate for its specific antigen, and will also depend on the rate at which an administered conjugate is depleted from the free volume other subject to which it is administered. Common ranges for therapeutically effective dosing of a conjugate disclosed herein may be, by way of nonlimiting example, from about 0.1 mg/kg body weight to about 50 mg/kg body weight, from about 0.1 mg/kg body weight to about 100 mg/kg body weight or from about 0.1 mg/kg body weight to about 150 mg/kg body weight. Common dosing frequencies may range, for example, from twice daily to once a month (e.g., once daily, once weekly; once every other week; once every 3 weeks or monthly). For example, HER2 targeted conjugates of the disclosure can be administered (e.g., as a single dose weekly, every 2 weeks, every 3 weeks, or monthly) at about 0.1 mg/kg to about 20 mg/kg (e.g., 0.2 mg/kg, 0.5 mg/kg, 0.67 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg). For example, conjugates of the disclosure can be administered (e.g., as a single dose weekly, every 2 weeks, every 3 weeks, or monthly) at about 0.1 mg/kg to about 20 mg/kg (e.g., 0.2 mg/kg, 0.5 mg/kg, 0.67 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg) for treating low HER2-expressing breast or low HER2-expressing gastric cancer.

Efficaciousness of treatment is determined in association with any known method for diagnosing or treating the particular HER2-related disorder. Alleviation of one or more symptoms of the HER2-related disorder indicates that the antibody confers a clinical benefit.

Methods for the screening of antibodies that possess the desired specificity include, but are not limited to, enzyme linked immunosorbent assay (ELISA) and other immunologically mediated techniques known within the art.

In another embodiment, HER2-targeted antibody-drug conjugates may be used in methods known within the art relating to the localization and/or quantitation of HER2 (e.g., for use in measuring levels of HER2 within appropriate physiological samples, for use in diagnostic methods, for use in imaging the protein, and the like). In a given embodiment, conjugates that comprise antibodies specific to HER2, or derivative, fragment, analog or homolog thereof, that contain the antibody derived antigen binding domain, are utilized as pharmacologically active compounds (referred to hereinafter as “Therapeutics”).

The HER2-targeted antibody-drug conjugates and/or immune checkpoint inhibitors thereof disclosed herein (also referred to herein as “active compounds”), can be incorporated into pharmaceutical compositions suitable for administration. Principles and considerations involved in preparing such compositions, as well as guidance in the choice of components are provided, for example, in Remington's Pharmaceutical Sciences: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Such compositions typically comprise the conjugates and/or immune checkpoint inhibitors and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated.

The formulations to be used for in vivo administration must be sterile. This is readily accomplished by filtration through sterile filtration membranes.

A pharmaceutical composition disclosed herein is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a conjugate disclosed herein) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile inj ectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a sustained/controlled release formulations, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

For example, the active ingredients can be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT T (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The materials can also be obtained commercially from Alza Corporation and Nova Pharmaceuticals, Inc. Liposomal suspensions (including liposomes targeted to infected cells with monoclonal antibodies to viral antigens) and can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms disclosed herein are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In one embodiment, the active compounds are administered in combination therapy, i.e., combined with other agents, e.g., therapeutic agents, that are useful for treating pathological conditions or disorders, such as various forms of cancer, autoimmune disorders and inflammatory diseases. The term “in combination” in this context means that the agents are given substantially contemporaneously, either simultaneously or sequentially. If given sequentially, at the onset of administration of the second compound, the first of the two compounds is, for example, still detectable at effective concentrations at the site of treatment.

For example, the combination therapy can include one or more conjugates disclosed herein co-formulated with, and/or co-administered with, one or more immune checkpoint inhibitors disclosed herein, and optionally one or more additional antibodies, e.g., a HER2 antibody, a HER2 dimerization inhibitor antibody or a combination of a HER2 antibody and a HER2 dimerization inhibitor antibody, such as, for example, trastuzumab, pertuzumab or a combination of trastuzumab and pertuzumab, or a biosimilar of trastuzumab and/or pertuzumab or combinations of biosimilars.

For example, the combination therapy can include one or more conjugates disclosed herein co-formulated with, and/or co-administered with, one or more immune checkpoint inhibitors disclosed herein, and optionally one or more additional therapeutic agents, e.g., a taxane (paclitaxel or docetaxel), an anthracycline (doxorubicin or epirubicin), cyclophosphamide, capecitabine, tamoxifen, letrozole, carboplatin, gemcitabine, cisplatin, erlotinib, irinotecan, fluorouracil, or oxaliplatin. Such combination therapies may advantageously utilize lower dosages of the administered therapeutic agents, thus avoiding possible toxicities or complications associated with the various monotherapies.

In some embodiments, the additional therapeutic agent(s) used in combination with a conjugate and immune checkpoint inhibitors disclosed herein are those agents that interfere at different stages in an immune and/or inflammatory response. In one embodiment, the combination of conjugate and checkpoint inhibitor described herein may be co-formulated with, and/or co-administered with, one or more additional agents.

In some embodiments, the immune checkpoint inhibitor provided herein is formulated in an amount for direct administration in a range between about 7.5 mg to about 5,000 mg, about 7.5 mg to about 1,500 mg, about 7.5 mg to about 750 mg, or about 22.5 to about 750 mg. In some examples, the immune checkpoint inhibitor can be formulated as a low dose formulation, for example, for more frequent administration. In such formulations, the immune checkpoint inhibitor is formulated for single dosage administration in an amount that is less than or less than about 1 mg, 500 μg, 400 μg, 300 μg, 200 μg, 100 μg, 50 μg, 30 μg, 20 μg, 10 μg, 5 μg or less than 1 μg. Thus, non-limiting amounts of immune checkpoint inhibitor that is formulated for direct administration include a dosage that is or is about 1 μg 5 μg, 10 μg, 20 μg, 30 μg, 50 μg, 100 μg, 200 μg 250 μg, 500 μg, 1 mg, 5 mg, 7.5 mg, 10 mg, 20 mg, 22, 5 mg, 30 mg, 35 mg, 37.5 mg, 50 mg, 75 mg, 100 mg, 150 mg, 200 mg, 210 mg, 250 mg, 300 mg, 350 mg, 375 mg, 500 mg, 750 mg, 1000 mg, 1500 mg, 2000 mg, 2500 mg, 3000 mg, 3500 mg, 4000 mg, 4500 mg, or 5000 mg.

Formulations containing an immune checkpoint inhibitor, such as an anti-immune checkpoint protein antibody can be provided as a percentage of weight per volume. Such concentrations of an immune checkpoint inhibitor include, but are not limited to, a concentration that is or is about 0.01% to 99.5% w/v, such as 0.1% to 90% w/v, 0.1% to 70% w/v, 0.1% to 30% w/v, or 5% to 22% w/v. In examples, the immune checkpoint inhibitor in compositions can be provided at a concentration that is from about 0.5 mg/mL to about 500 mg/mL, such as 0.5 mg/mL to 250 mg/mL, 0.5 mg/mL to 100 mg/mL, 0.5 mg/mL to 50 mg/mL, 0.5 mg/mL to 10 mg/mL, 0.5 mg/mL to 6 mg/mL, 0.5 mg/mL to 2 mg/mL, 2 mg/mL to 250 mg/mL, 2 mg/mL to 100 mg/mL, 2 mg/mL to 50 mg/mL, 2 mg/mL to 10 mg/mL, 2 mg/mL to 6 mg/mL, 6 mg/mL to 250 mg/mL, 6 mg/mL to 100 mg/mL, 6 mg/mL to 50 mg/mL, 6 mg/mL to 10 mg/mL, 10 mg/mL to 250 mg/mL, 10 mg/mL to 100 mg/mL, 10 mg/mL to 50 mg/mL, 50 mg/mL to 250 mg/mL, 50 mg/mL to 100 mg/mL, or 100 mg/mL to 250 mg/mL. For example, the immune checkpoint inhibitor can be provided in the composition at a concentration that is at least 0.5 mg/mL, 1 mg/mL, 2 mg/mL, 5 mg/mL, 6 mg/mL, 7 mg/mL, 8 mg/mL, 9 mg/mL, 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 120 mg/mL, 150 mg/mL, 180 mg/mL, 200 mg/mL, 220 mg/mL, 250 mg/mL or more. In some cases, the immune checkpoint inhibitor in the formulation is provided in an amount that is at least 1% (10 mg/mL) to 30% (300 mg/mL), for example, at least 1%, 2%, 3%, 4%, 5%, 6%, 7%, 8%, 9%, 10%, 11%, 12%, 13%, 14%, 15%, 16%, 17%, 18%, 19%, 20% 21%, 22%, 23%, 24%, 25%, 26%, 27%, 28%, 29%, 30%, or more.

The volume of the composition can be about 0.5 mL to about 1000 mL, such as 0.5 mL to 100 mL, 0.5 mL to 10 mL, 1 mL to 500 mL, 1 mL to 10 mL, such as about 0.5 mL, 1 mL, 2 mL, 3 mL, 4 mL, 5 mL, 6 mL, 7 mL, 8 mL, 9 mL, 10 mL, 15 mL, 20 mL, 30 mL, 40 mL, 50 mL, 60 mL, 70 mL, 80 mL, 90 mL or more. When administered, the composition can be administered by infusion. For larger volumes, the time of infusion can be adapted to facilitate delivery of the larger volume. For example, infusion time can be at least 1 minute, 5 minutes, 10 minutes, 20 minutes, 30 minutes, 40 minutes, 50 minutes, 1 hour, 2 hours or more.

The antibody preparations provided herein can be formulated as pharmaceutical compositions for single or multiple dosage use. Typically, the antibody is formulated in an amount such that it is ready to use and that no further dilution is necessary. Depending on whether the formulation is provided as a single or multiple dosage formulation, one of skill in the art can empirically determine the exact amount of antibody in the formulation.

It is understood that antibody formulations can contain other components, including carriers, polymers, lipids and other excipients. The dosages concentrations above are with respect to the antibody component, which is the active ingredient.

### Dosage and Administration

The combination therapy provided herein, containing a HER2-targeted antibody-drug conjugate and an immune checkpoint inhibitor, such as an anti-immune checkpoint protein antibody (e.g., an anti-CTLA4 or anti-PD-1 antibody), is administered in an amount sufficient to exert a therapeutically useful effect. Typically, the active agents are administered in an amount that does not result in undesirable side effects of the patient being treated, or that minimizes or reduces the observed side effects as compared to dosages and amounts required for single treatment with one of the above agents. For example, the combination therapy comprising a HER2-targeted antibody-drug conjugate and an immune checkpoint inhibitor, such an anti-immune checkpoint protein antibody, results in decreased tumor progression compared to administration of vehicle or either agent alone. Thus, it is possible, the amount of an immune checkpoint inhibitor, such as an anti-immune checkpoint protein antibody, that can be administered in the combination therapy provided herein, compared to the amount of the immune checkpoint inhibitor (e.g., anti-immune checkpoint protein antibody) administered alone or using a known method is reduced, while achieving substantially the same or improved therapeutic efficacy. By virtue of the decreased dosage that can be administered, side effects associated with anti-immune checkpoint protein antibody administration, such as immune-related adverse events, described elsewhere or herein, are reduced, minimized or avoided.

It is within the level of one of skill in the art to determine the precise amounts of active agents, including HER2-targeted antibody-drug conjugates and immune checkpoint inhibitors to be administered to a subject. For example, such agents and uses for treating cancers and solid tumors, are well-known in the art. Thus, dosages of such agents in a combination therapy can be chosen based on standard dosing regimens for that agent under a given route of administration.

It is understood that the precise dosage and duration of treatment is a function of the tissue or tumor being treated and may be determined empirically using known testing protocols or by extrapolation from in vivo or in vitro test data and/or can be determined from known dosing regimens of the particular agent. It is to be noted that concentrations and dosage values may also vary with the age of the individual treated, the weight of the individual, the route of administration and/or the extent or severity of the disease and other factors that are within the level of a skilled medical practitioner to consider. Generally, dosage regimens are chosen to limit toxicity. It should be noted that the attending physician would know how to and when to terminate, interrupt or adjust therapy to lower dosage due to toxicity, or bone marrow, liver or kidney or other tissue dysfunctions. Conversely, the attending physician would also know how to and when to adjust treatment to higher levels if the clinical response is not adequate (precluding toxic side effects). It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope thereof.

For example, the HER2-targeted antibody-drug conjugate, is administered in a therapeutically effective amount to decrease the tumor volume.

The amount of a HER2-targeted antibody-drug conjugate administered for the treatment of a disease or condition, for example a cancer or solid tumor can be determined by standard clinical techniques. In addition, in vitro assays and animal models can be employed to help identify optimal dosage ranges. The precise dosage, which can be determined empirically, can depend on the route of administration, the type of disease to be treated and the seriousness of the disease.

In examples herein, the immune checkpoint inhibitor, such as an anti-immune checkpoint protein antibody, is provided in a therapeutically effective amount for the particular dosage regimen. Therapeutically effective concentrations can be determined empirically by testing the compounds in known in vitro and in vivo systems, such as the assays provided herein. The concentration of a selected immune checkpoint inhibitor in the composition depends on absorption, inactivation and excretion rates of the complex, the physicochemical characteristics of the complex, the dosage schedule, and amount administered as well as other factors known to those of skill in the art.

The amount of a selected immune checkpoint inhibitor to be administered for the treatment of cancers can be determined by standard clinical techniques or other methods as described herein. In addition, in vitro assays and animal models can be employed to help identify optimal dosage ranges. Hence, the precise dosage, which can be determined empirically, can depend on route of administration, the type of cancer to be treated and the progression of the disease. Exemplary dosage regimens (doses and frequencies) of immune checkpoint inhibitor formulations for treating cancers are provide below. Other dosage regimens are well-known to those of skill in the art. If necessary, a particular dosage and duration and treatment protocol can be empirically determined or extrapolated.

In some examples, the dose of an immune checkpoint inhibitor is a function of immune cell populations. For example, the dose of the immune checkpoint inhibitor can be modulated to minimize the increase in the number of T_{reg} cells in response to the administered agent. For example a maximum dose can be determined to be the maximum dose that does not result in an increase in the number of circulating T_{reg} cells. In another example, the dose of an immune checkpoint inhibitor can be modulated to maximize the increase in the number of effector cells in the tumor-bearing subject. In a further example, the dose of an immune checkpoint inhibitor is selected that minimizes or prevents and increase in the number of T_{reg} cells, but maximizes the increase in the number of effector cells. Methods for determining such doses are known in the art and described herein. For example, the number(s) of T_{reg} cells and/or effector cells can be measured by flow cytometry (described herein above) at one or more different time points after administration of the immune checkpoint inhibitor. For examples the number(s) of T_{reg} cells and/or effector cells can be determined on the same day as administration and/or 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks or more after administration of the immune checkpoint inhibitor, such as 1 day, 2 days, 3 days, 4 days, 5 days, 6 days, or 1 week after administration of the immune checkpoint inhibitor. The following dose can be modulated to achieve the desired affects with respect to the levels of T_{reg} cells and/or effector cells detected.

In some examples, exemplary doses of intravenously administered immune checkpoint inhibitor, such as an anti-immune checkpoint protein antibody, can be used as a starting point to determine appropriate dosages. Dosage levels can be determined based on a variety of factors, such as body weight of the individual, general health, age, the activity of the specific compound employed, sex, diet, time of administration, rate of excretion, drug combination, the severity and course of the disease, and the patient's disposition to the disease and the judgment of the treating physician. Non-limiting exemplary dosages of the provided immune checkpoint inhibitors are from about 0.1 mg per kg body weight (mg/kg BW) to about 50 mg/kg BW, such as about 0.1 mg/kg to about 20 mg/kg BW, about 0.1 mg/kg to about 10 mg/kg BW, about 0.3 mg/kg to about 10 mg/kg, about 0.5 mg/kg to 5 mg/kg or 0.5 mg/kg to 1 mg/kg. For example, the immune checkpoint inhibitor can be administered to tumor-bearing animals in doses of, for example, at least about 0.1 mg/kg, 0.15 mg/kg, 0.2 mg/kg, 0.25 mg/kg, 0.30 mg/kg, 0.35 mg/kg, 0.40 mg/kg, 0.45 mg/kg, 0.5 mg/kg, 0.55 mg./kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 30 mg/kg, 40 mg/kg, 50 mg/kg, or more. In particular, the immune checkpoint inhibitor is administered at a dose of at least 0.1 mg/kg, 0.3 mg/kg, 1 mg/kg, 3, mg/kg, 5 mg/kg, 7.5 mg/kg, 10 mg/kg, or 15 mg/kg. In some examples, exemplary dosages include, but are not limited to, about 0.01 mg/m² to about or 500 mg/m², such as for example, about or 0.01 mg/m², about or 0.1 mg/m², about or 0.5 mg/m², about or 1 mg/m², about or 5 mg/m², about or 10 mg/m², about or 15 mg/m², about or 20 mg/m², about or 25 mg/m², about or 30 mg/m², about or 35 mg/m², about or 40 mg/m², about or 45 mg/m², about or 50 mg/m², about or 100 mg/m², about or 150 mg/m², about Or 200 mg/m², about or 250 mg/m², about or 300 mg/m², about or 400 mg/m², about or 500 mg/m². It is understood that one of skill in the art can recognize and convert dosages between units of mg/kg and mg/m² (see, e.g., Michael J. Derelanko, TOXICOLOGIST'S POCKET HANDBOOK, CRC Press, p. 16 (2000)).

It is understood that the amount to administer will be a function of the type of cancer being treated, the route of administration, and the tolerability of possible side effects. If necessary, dosage can be empirically determined. To achieve such dosages, volumes of immune checkpoint inhibitor-containing formulations administered subcutaneously can range from about 1 mL to 700 mL, for example, 10 mL to 500 mL, such as 100 mL to 400 mL. For example, volumes of immune checkpoint inhibitor-containing formulations administered subcutaneously can be about 1 mL, 2 mL, 3 mL, 4 mL, 5 mL, 10 mL, 20 mL, 30 mL, 40 mL, 50 mL, 100 mL, 200 mL, 300 mL, 400 mL, 500 mL, 600 mL, 700 mL or more for single dosage administration.

In other examples, the dose of the immune checkpoint inhibitor is a low dose, such as less than or equal to 1 mg per administration, for example, less than or equal to 500 jag, 400 jag, 300 jag, 200 jag, 100 jag, 50 jag, 30 jag, 20 jag, 10 jag, 5 jag or 1 jag per administration. It will be appreciated that such low doses can be administered in a suitable volume repeatedly to the patient over time, for example twice daily, once daily, once every other day, twice weekly, once weekly, bimonthly, monthly, etc.

The conjugates and/or immune checkpoint inhibitor formulations provided herein can be administered intravenously, subcutaneously, intratumorally, intradermally, orally or by other routes of administration. The particular route can differ, between the administered agents or can be the same. For example, one or more, or all of the agents used in the combination therapy, can be administered intravenously. In some examples, a conjugate is administered intravenously and the immune checkpoint inhibitor is administered intravenously.

For intravenous administration, one or more, or all, of the agents used in the combination therapy can be administered by push or bolus, by infusion, or via a combination thereof. The infusion time can be about 1 minute to three hours, such as about 1 minute to about two hours, or about 1 minute to about 60 minutes, or at least 10 minutes, 40 minutes, or 60 minutes. The agents can be administered by concurrent infusion or by subsequent infusion. For example, the administered agents are administered separately and are provided in separate bags for separate infusions. In particular examples, the HER2-targeted antibody-drug conjugate composition and the immune checkpoint inhibitor composition are formulated and administered separately.

The HER2-targeted antibody-drug conjugate can be administered prior to, simultaneously with or near simultaneously with, sequentially with or intermittently with the immune checkpoint inhibitor. For example, the HER2-targeted antibody-drug conjugate and the immune checkpoint inhibitor, e.g., an anti-immune checkpoint protein antibody (e.g., an anti-CTLA4 or anti-PD-1 antibody) can be co-administered or separately.

In one embodiment, the HER2-targeted antibody-drug conjugate is administered prior to the immune checkpoint inhibitor. For example, the HER2-targeted antibody-drug conjugate is administered up to about 48 hours prior to administering the immune checkpoint inhibitor. For example, the HER2-targeted antibody-drug conjugate is administered about 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours, or up to about 48 hours prior to administration of the immune checkpoint inhibitor.

In other embodiments, the HER2-targeted antibody-drug conjugate is administered after the immune checkpoint inhibitor. For example, the HER2-targeted antibody-drug conjugate is administered up to about 48 hours after administering the immune checkpoint inhibitor. For example, the HER2-targeted antibody-drug conjugate is administered about 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours, or up to about 48 hours after administration of the immune checkpoint inhibitor.

The frequency and timing of administration, and the dosage amounts, can be administered periodically over a cycle of administration to maintain a continuous and/or long term effect of the active agents for a desired length of time and need not be the same for the HER2-targeted antibody-drug conjugate and immune checkpoint inhibitor. The provided compositions of each active agent or combinations thereof can be administered hourly, daily, weekly, monthly, yearly or once. The length of time of the cycle of administration can be empirically determined, and is dependent on the disease to be treated, the severity of the disease, the particular patient, and other considerations within the level of skill of the treating physician. The length of time of treatment with a combination therapy provided herein can be one week, two weeks, one months, several months, one year, several years or more.

For example, the frequency of administration of the HER2-targeted antibody-drug conjugate is once a day, every other day, twice weekly, once weekly, once every 2 weeks, once every 3 weeks or once every 4 weeks. The dosages can be divided into a plurality of cycles of administration during the course of treatment. For example, the HER2-targeted antibody-drug conjugate can be administered at the frequency over a period of about a month, 2 months, 3 months, 4 months, 5 months, 6 months, a year or more. The frequency of administration can be the same throughout the period of the cycle or can differ. For example, an exemplary dosage frequency is two times a week at least for a first week of a cycle of administration. After the first week, the frequency can continue at twice a week, can increase to more than twice a week, or can be reduced to no more than once a week. It is within the level of a skilled person to determine the particular dosage frequency and cycle of administration based on the particular dosage being administered, the disease or condition being treated, the severity of the disease or condition, the age of the subject and other similar factors.

The immune checkpoint inhibitor can be administered at the same frequency or at a different frequency. For example, each administration of the immune checkpoint inhibitor is preceded by an administration of the HER2-targeted antibody-drug conjugate by not more than 48 hours. For example, each dose of the HER2-targeted antibody-drug conjugate is followed 24 to 48 hr later by a dose of immune checkpoint inhibitor. In certain embodiments, the immune checkpoint inhibitor is administered less frequently than the HER2-targeted antibody-drug conjugate, but each dose of immune checkpoint inhibitor is preceded by a dose of the HER2-targeted antibody-drug conjugate. For example, the immune checkpoint inhibitor is administered twice weekly, once weekly, once every 2 weeks, once every 3 weeks, once every 4 weeks, once every 6 weeks, once every 2 months, once every 3 months, once every 4 months, once every 5 months, or once every 6 months, and in a manner that is preceded by administration of a HER2-targeted antibody-drug conjugate. In another example, each dose of the HER2-targeted antibody-drug conjugate is preceded by a dose of immune checkpoint inhibitor. In certain embodiments, the immune checkpoint inhibitor is administered more frequently than the HER2-targeted antibody-drug conjugate. For example, the immune checkpoint inhibitor is administered twice weekly, once weekly, once every 2 weeks, once every 3 weeks, once every 4 weeks, once every 6 weeks, once every 2 months, once every 3 months, once every 4 months, once every 5 months, or once every 6 months, and in a manner that some but not all checkpoint inhibitor dosages are followed by administration of a HER2-targeted antibody-drug conjugate.

If disease symptoms persist in the absence of discontinued treatment, treatment can be continued for an additional length of time. Over the course of treatment, evidence of disease and/or treatment-related toxicity or side effects can be monitored.

The cycle of administration of the HER2-targeted antibody-drug conjugate and/or immune checkpoint inhibitor can be tailored to add periods of discontinued treatment in order to provide a rest period from exposure to the agents. The length of time for the discontinuation of treatment can be for a predetermined time or can be empirically determined depending on how the patient is responding or depending on observed side effects. For example, the treatment can be discontinued for one week, two weeks, one month or several months. Generally, the period of discontinued treatment is built into a cycle of dosing regimen for a patient.

An exemplary dosing regimen is a treatment cycle or cycle of administration of 28 days. The agent, such as the HER2-targeted antibody-drug conjugate disclosed herein, can be administered on day 1, followed by administration of an immune checkpoint inhibitor of the disclosure, such as an immune checkpoint protein antibody on day 2, followed by 26 days without dosing. In another example, the HER2-targeted antibody-drug conjugate can be administered twice weekly, on days 1, 4, 8, 11, 15, 18, 22 and 25, and the immune checkpoint inhibitor is administered once on day 2. In another example the HER2-targeted antibody-drug conjugate is administered twice weekly, on days 1, 4, 8, 11, 15, 18, 22 and 25 and the immune checkpoint inhibitor also is administered twice weekly on days 2, 5, 9, 12, 16, 19, 23, and 26. It is understood that the above description is for exemplification purposes only and that variations of the above can be employed. Further, similar cycles of administration can be applied to all administered agents, or each administered agent can be employed in its own dosing regimen in the combination therapy provided herein.

It is within the level of one of skill in the art to determine the precise cycle of administration and dosing schedule. As noted above, the cycle of administration can be for any desired length of time. Hence, the 28-day cycle of administration can be repeated for any length of time. It is within the level of skill of the treating physician to adopt a cycle of administration and dosing regimen that meets the needs of the patient depending on personal considerations specific to the patient and disease to be treated.

### Diagnostic and Prophylactic Formulations

The conjugates and immune checkpoint inhibitors disclosed herein are used in diagnostic and prophylactic formulations. In one embodiment, a HER2-targeted antibody-drug conjugate and an immune checkpoint inhibitor disclosed herein are administered to patients that are at risk of developing one or more of the aforementioned diseases, such as for example, without limitation, cancer. A patient's or organ's predisposition to one or more of the aforementioned indications can be determined using genotypic, serological or biochemical markers.

In another embodiment, a HER2-targeted antibody-drug conjugate and an immune checkpoint inhibitor disclosed herein are administered to human individuals diagnosed with a clinical indication associated with one or more of the aforementioned diseases, such as for example, without limitation, cancer. Upon diagnosis, a HER2-targeted antibody-drug conjugate and an immune checkpoint inhibitor disclosed herein are administered to mitigate or reverse the effects of the clinical indication associated with one or more of the aforementioned diseases.

In another embodiment, a method for identifying a breast cancer patient amenable to treatment with the combinations of conjugates and immune checkpoint inhibitors disclosed herein, comprise measuring the status of certain characteristics in a tumor sample obtained from the patient, and identifying the patient for treatment based on the status of certain characteristics in the tumor sample.

Antibodies disclosed herein are also useful in the detection of HER2 in patient samples and accordingly are useful as diagnostics. For example, HER2 antibodies disclosed herein are used in in vitro assays, e.g., ELISA, to detect HER2 levels in a patient sample.

In one embodiment, a HER2 antibody disclosed herein is immobilized on a solid support (e.g., the well(s) of a microtiter plate). The immobilized antibody serves as a capture antibody for any HER2 that may be present in a test sample. Prior to contacting the immobilized antibody with a patient sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of HER2 antigen in the test sample is determined by comparison with a standard curve developed from the standard samples.

It will be appreciated that based on the results obtained using the HER2 antibodies disclosed herein in an in vitro diagnostic assay, it is possible to stage a disease in a subject based on expression levels of the HER2 antigen. For a given disease, samples of blood are taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples are illustrative and are not intended to be limiting and it will be readily understood by one of skill in the art that other reagents or methods may be utilized.

### ABBREVIATIONS

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples.
AF-HPA Auristatin F-hydroxypropyl amide
Ala Alanine
BA β-Alanine
DAR Drug:Antibody ratio
DAMPs Damage-associated molecular patterns
EG2 Diethylene glycol
FBS Fetal bovine serum
ICD Immunogenic cell death
IP Intraperitoneal
IV Intravenous
MI Maleimide or maleimido
PBS Phosphate buffered saline
PHF poly(1-hydroxymethylethylene hydroxylmethylformal)
q“m” dx“n” Dosing frequency of every “m” days for “n” cycles
q“m” wx“n” Dosing frequency of every “m” weeks for “n” cycles

### General Information

XMT 1519-(EG2-MI-(7.7 kDa PHF-BA-(AF-HPA-Ala))) conjugate (XMT-1519 conjugate) was prepared as described in US Application No. 20150366987(A1).

AF-HPA was prepared as described in U.S. Pat. No. 8,685,383(B2)

CDRs were identified by the Kabat numbering scheme.

Tumor growth inhibition (% TGI) was defined as the percent difference in median tumor volumes (MTVs) between treated and control groups.

Treatment efficacy was determined from the incidence and magnitude of regression responses of the tumor size observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm³ for three consecutive measurements during the course of the study. An animal with a CR response at the termination of a study was additionally classified as a tumor-free survivor (TFS). Animals were monitored for regression responses.

### Example 1. The Antitumor Activity of XMT 1519-(EG2-MI-(7.7 kDa PHF-BA-(AF-HPA-Ala))) Conjugate (XMT-1519 Conjugate) in Combination with Pembrolizumab (Keytruda) in Low Passage TumorGraft™ Model of Non-Small Cell Lung Carcinoma in Humanized Mice (CTG-0860)

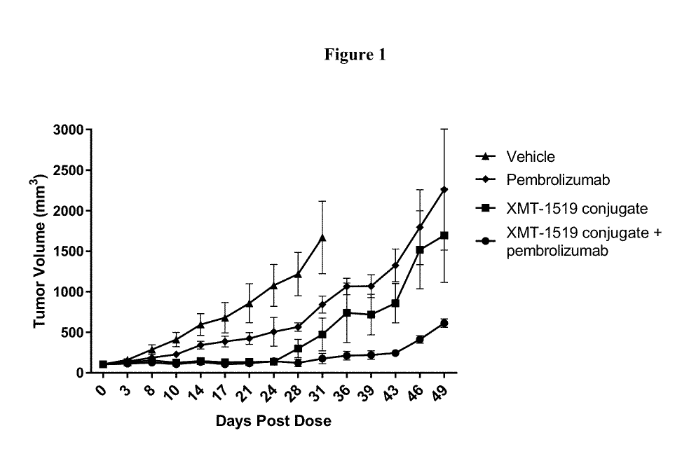

Unmatched human leukocyte antigen (HLA) humanized CD34+ engrafted immune compromised female mice (Taconic NOG) were subcutaneously implanted unilaterally on the left flank with tumor fragments (n=5 for each group and two additional mice for tumor infiltration lymphocytes measurement for the test compounds only). Test compounds (XMT-1519 conjugate, DAR 12.2; pembrolizumab; and combination of XMT-1519 conjugate, DAR 12.2, and pembrolizumab) or vehicle were administered as indicated in Table 1. Tumor size was measured at the times indicated in FIG. 1 using digital calipers. Tumor volume was calculated and was used to determine the tumor growth inhibition. Control mice were sacrificed when tumors reached a size of 1500 mm³. Tumor volumes are reported as the mean±SEM for each group.

When tumors reached a size of 1500 mm³ in control mice, they were sacrificed as well as two mice from each treatment groups, and tumor infiltration lymphocytes were analyzed. At the study endpoint (day 49) all the surviving mice were sacrificed and tumor infiltration lymphocytes were analyzed. Tumors were dissociated and analyzed for tumor infiltrating lymphocytes by flow cytometry for the following markers: human CD45 infiltrating lymphocytes, hCD3 (T cells), hCD4 (helper T cells), hCD8 (cytotoxic T cells), hCD19 (B cells), T cells activation and proliferation—hCD25. There was no clear correlation observed between tumor infiltrating lymphocytes and tumor response.

TABLE 1

| | | Dose | | |
|---|---|---|---|---|
| | Dose | Volume | Route of | |
| Agent | (mg/kg) | (mL/kg) | Administration | Schedule |
| Vehicle | — | 5 | IP | q4dx6 |
| (Saline) | | | | |
| XMT-1519 | 1.0 | 10 | IV | q7dx3 |
| conjugate | | | | |
| Pembrolizumab | 2.5 | 5 | IP | q5dx6 |
| Pembrolizumab + | 2.5 | 5 | IP | q5dx6 |
| XMT-1519 | 1.0 | 10 | IV | q7dx3 |
| conjugate | | | | |

FIG. 1 provides the results for the tumor response in humanized mice subcutaneously implanted with tumor fragments (n=5 for each group) after administration of vehicle; XMT-1519 conjugate; pembrolizumab; and combination of XMT-1519 conjugate and pembrolizumab each as administered as outlined in Table 1. XMT-1519 conjugate as well as pembrolizumab each showed a decrease in tumor volume when administered as single agents. The combination of XMT-1519 conjugate and pembrolizumab was most efficacious in inhibiting tumor growth.

### Example 2. ATP Release from Cells to Intracellular Space (Media) Treated with AF-HPA and XMT-1519 Conjugate

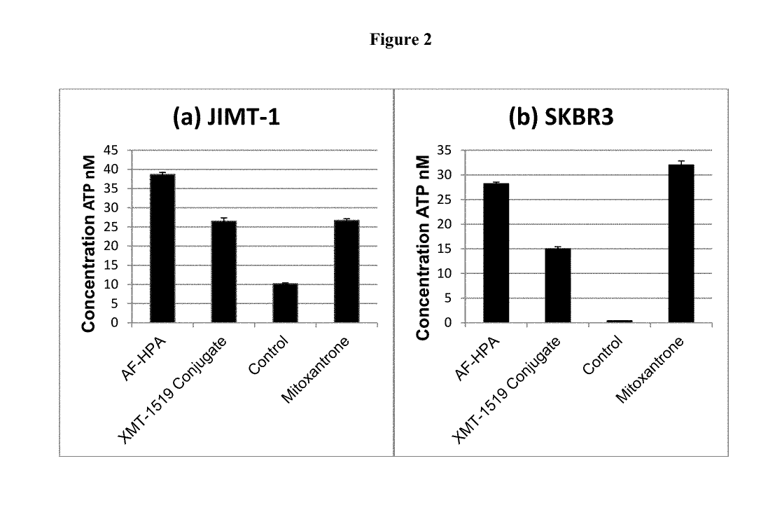

Cancer cells undergoing apoptosis in response to specific anticancer therapies are immunogenic (also known as immunogenic cell death (ICD)), as long as they emit precise DAMPs in a spatiotemporally defined fashion. To demonstrate that AF-HPA and XMT-1519 conjugate induce ICD, associated with DAMPs signal production, ATP release from cells was evaluated. Mitoxantrone, a known strong inducer of ICD and ATP release, was used as a positive control. Briefly, two HER2 expressing cell lines, JIMT-1 (cat. # ACC589, DSMZ Cell Collection) and SKBR3 (cat # ATCC® HTB 30™, American Tissue Culture Collection) were seeded at a density of 7500 cells per well in 24 wells plate, allowed to grow for 24 h, and then treated with AF-HPA or XMT-1519 conjugate at 0.5 μM in 100 μl in RPMI 1860 Media (Cat#11875-119, Thermo Fisher Scientific) for 24 h. The cells were then pelleted by centrifugation and ATP release was measured using ENLITEN® ATP Assay System (Promega) according to manufacturer's instructions. FIG. 2 shows that ATP was released in the cell lines after treatment with mitoxantrone, AF-HPA and XMT-1519 conjugate compared to the untreated (control) cells.

### Example 3. Calreticulin Exposure on Cell Membrane in Various Cell Lines after Treatment with AF-HPA or XMT-1519 Conjugate

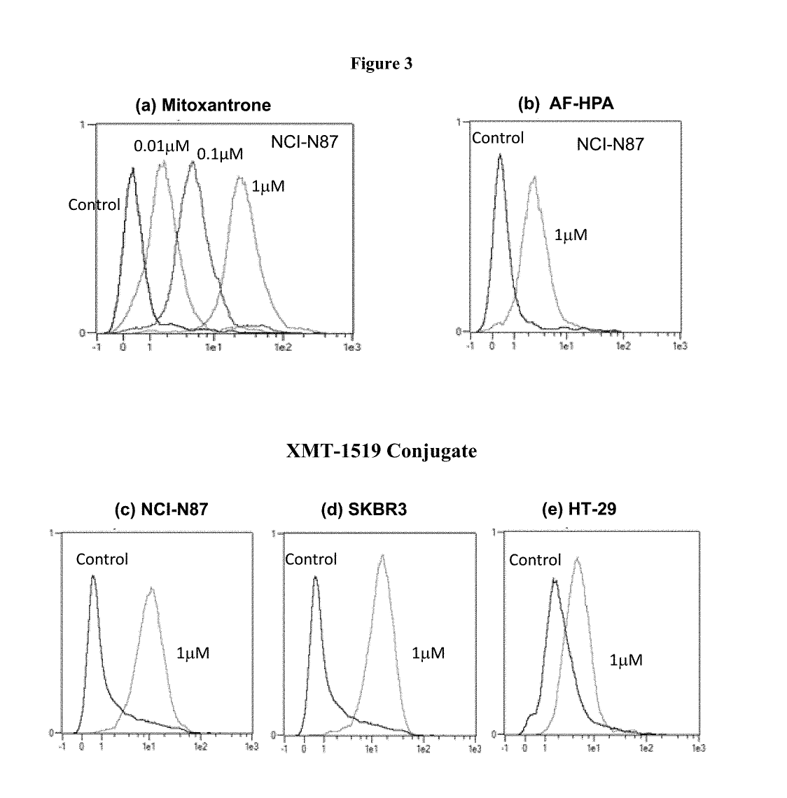

To demonstrate that AF-HPA and XMT-1519 conjugate induce ICD, associated with DAMPs signal production, calreticulin exposure on cell membrane was evaluated. High HER2-expressing cell lines, NCI-N87 (800,000 HER2 receptors), SKBR3 (700,000 HER2 receptors) and low HER2 expressing cell line HT-29 (16,000 HER2 receptors) were re-suspended at a density of 2×10⁶ cells/mL in 100 μl PBS containing 2% FBS. Cells in 96 well plate (Corning® 96 Well Clear Round Bottom Polypropylene Not Treated Microplate cat#3879) were treated with XMT-1519 conjugate or AF-HPA at 1 μM or with mitoxantrone, a strong calreticulin exposure inducer, at 0.01 to 1 μM for 2.5 h at 37° C. Cells were then incubated with anti-calreticulin antibody (clone 16B11.1 Cat: MABT217, Millipore Sigma, 1:200) in ice cold PBS containing 2% FBS for 1 h on ice, washed twice with cold PBS containing 2% and then incubated for 20 min on ice with a secondary antibody (Alexa 647-anti Mouse IgG (H+L) 1:800, Thermo Fisher Scientific Cat#:A32728) in ice cold PBS containing 2% FBS. Thereafter the cells were stained with Annexin V (Pacific Blue™ cat# A35122, Thermo Fisher Scientific) according to manufacturer's instructions to identify apoptotic cell population. Necrotic cell population was labeled by propidium iodide (cat# P3566 Thermo Fisher Scientific) at 1:1000 dilution. Cells were then analyzed by flow cytometry using MACSQuant® Analyzer 10. Apoptotic and necrotic cell populations were excluded and only live cells for measured for calreticulin exposure. As shown in FIG. 3, mitoxantrone resulted in a dose dependent calreticulin exposure in NCI-N87 cells (panel (a) of FIG. 3). AF-HPA induced calreticulin exposure in NCI-N87 cells (panel (b) of FIG. 3). XMT-1519 conjugate induced calreticulin exposure in all three cell lines (panels (c)-(e) of FIG. 3), where the most pronounced effect was observed in high HER2 expressing cells NCI-N87 and SKBR3.

### Example 4. Generation of a4T-7bb7 Cell Line

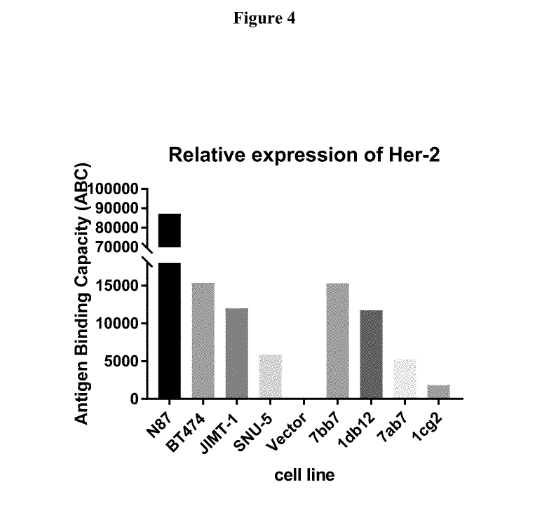

The 4T1 cell line (a mouse triple negative breast cancer cell line) was transduced with human HER2 using the lentiviral vector, HER2_FL_EOm_UT_pcDNA3.4 with a neomycin resistance selection gene. The transduced cells were selected using 0.25 mg/mL of the antibiotic, G418, in the culture medium and sub-cloned by limited dilution to generate four different human Her-2 expressing clones, i.e. 7bb7, 1db12, 7ab7 and 1cg2. The expression level of human Her-2 was tested in these clones using flow cytometry, and compared to that of different human Her-2+ cancer cell line (N87, BT474, JIMT-1 and SNU-5). FIG. 4 shows the relative Her-2 expression levels (e.g., antigen binding capacity) in the different human and mouse transgenic cell lines. Clone 4T1-7bb7, which expressed the highest level of human Her-2, was used to developed a stable in vivo syngeneic human Her-2 expressing mouse model, to test the in vivo efficacy and immunological mechanisms of XMT-1519 conjugate in a fully immune competent host.

### Example 5. Generation of a 4T1-7bb7 Syngeneic Mouse Model

Six to eight week old female, Balb/c mice (Jackson Labs, Bar Harbor, Me.) were subcutaneously implanted unilaterally on the left flank with 4×10⁴/mouse 4T1-7bb7 cells (n=13 mice/group). When tumors reached an average volume of 50+80 mm3, test compounds, XMT-1519 conjugate DAR—12.6, Kadcyla DAR—4.3 (Roche), anti-mouse PD1 mAb (clone RMP-1, Bio-X-cell, Lebanon, N.H.), anti-mouse CTLA4 (clone 9H10, Bio-X-cell, Lebanon, N.H.), and vehicle either alone or in different combinations were administered to the tumor bearing mice, using regimens shown in Table 2. The doses of XMT-1519 conjugate and Kadcyla were DAR matched such that the total amount of the conjugated drug was similar between the two treatments. Tumor sizes were measured twice a week using digital calipers, and average tumor volume was calculated to determine tumor growth inhibition. Control mice were sacrificed when tumors reached a size of 1500 mm³. Tumor volumes are reported as the mean±SEM for each group.

TABLE 2

| Treatment groups | | | |
|---|---|---|---|
| (Drug) | Dose | Schedule | Route |
| Vehicle (0.9% | Not applicable | qwx2 | IV |
| Saline) | | | |
| XMT-1519 | 4.0 mg/kg | qwx2 | IV |
| conjugate | | | |
| Kadcyla | 15.0 mg/kg | qwx2 | IV |
| Anti-PD1 mAb | 10.0 mg/kg | qd at days 0, 2, 4, 7, | IP |
| | | 10 | |
| Anti-CTLA-4 | 10.0 mg/kg | qd at days 0, 2, 4, 7, | IP |
| | | 10 | |
| XMT-1519 | 4.0 mg/kg + | qwx2 + qd at days 0, | IV + IP |
| conjugate + anti- | 10.0 mg/kg | 2, 4, 7, 10 | |
| PD1 mAb | | | |
| XMT-1519 | 4.0 mg/kg + | qwx2 + qd at days 0, | IV + IP |
| conjugate + CTLA-4 | 10.0 mg/kg | 2, 4, 7, 10 | |
| Kadcyla + anti-PD1 | 15.0 mg/kg + | qwx2 + qd at days 0, | IV + IP |
| mAb | 10.0 mg/kg | 2, 4, 7, 10 | |
| Kadcyla + CTLA-4 | 15.0 mg/kg + | qwx2 + qd at days 0, | IV + IP |
| | 10.0 mg/kg | 2, 4, 7, 10 | |

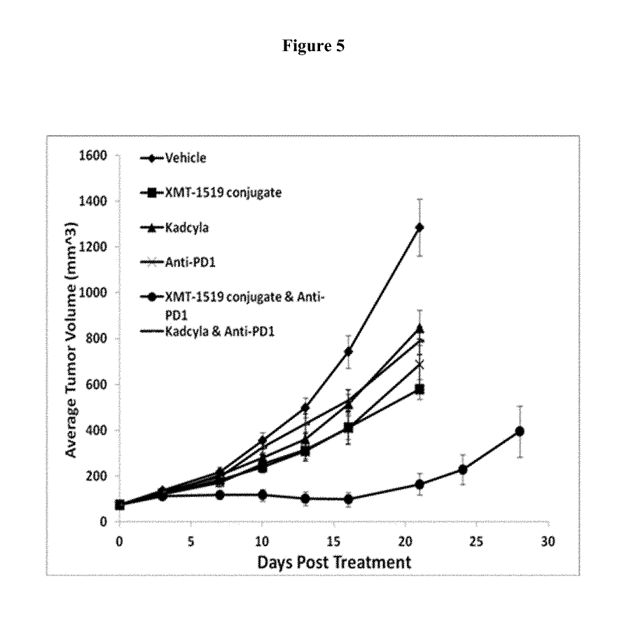

FIG. 5 shows the tumor response in mice after treatment with different regimens shown in Table 2. Treatment in the immunogenic tumor model with XMT-1519 conjugate or anti-PD1 as single agents showed significant inhibition of tumor growth in vivo. Importantly, a combination of anti-PD1 mAb and XMT-1519 conjugate, but not Kadcyla and anti-PD1 therapy, substantially and synergistically enhanced the anti-tumor efficacy, resulting in a complete response (CR) in one mouse.

### Example 6: Sequential Versus Concurrent Dosing of XMT-1519 Conjugate in Combination with an Anti-PD1 mAb in a Human Her2-Expressing Mouse 4T1-Breast Cancer Model in Immune-Competent Balb/c Mice

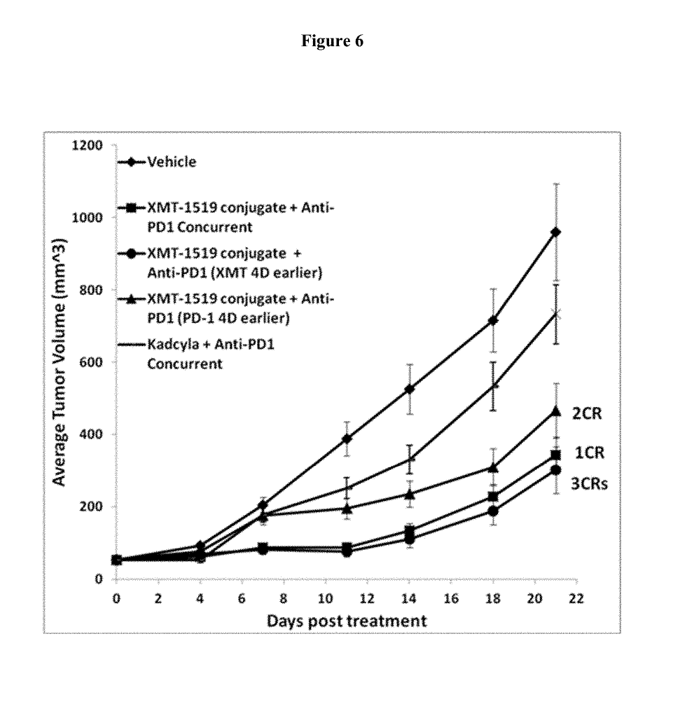

The therapeutic efficacy of XMT-1519 conjugate alone or in combination with an anti-mouse PD1 mAb was tested using sequential versus concurrent regimens in the 4T1-7bb7 syngeneic breast cancer model as described Example 5. Six to eight week old female, Balb/c mice (Jackson Labs, Bar Harbor, Me.) were subcutaneously implanted unilaterally on the left flank with 4×10⁴/mouse 4T1-7bb7 cells (n=12 mice/group). When tumors reached an average volume of 50+80 mm³ test compounds, XMT-1519 conjugate DAR—12.6, Kadcyla DAR-4.3 (Roche), anti-mouse PD1 mAb (clone RMP-1, Bio-X-cell, Lebanon, N.H.) and vehicle, either alone or in different combinations, were administered to the tumor bearing mice, using regimens as shown in Table 3. Notably, the combinations of XMT-1519 conjugate and anti-PD1 mAb were administered either concurrently, i.e. both therapies starting at the same time, or sequentially, i.e. the start date of one therapy was delayed by four days compared to that of the other therapy. Tumor sizes were measured twice a week as shown in FIG. 6 using digital calipers, and average tumor volume was calculated to determine tumor growth inhibition. Control mice were sacrificed when tumors reached a size of 1500 mm³. Tumor volumes are reported as the mean±SEM for each group.

TABLE 3

| Treatment groups | | | |
|---|---|---|---|
| (Drug) | Dose | Regimen | Route |
| Vehicle (0.9% | Not applicable | qwx2 | IV |
| saline) | | | |
| XMT-1519 | 4.0 mg/kg | qwx2 | IV |
| conjugate | | | |
| Kadcyla | 15.0 mg/kg | qwx2 | IV |
| Anti-PD-1 mAb | 10.0 mg/kg | qd at days 0, 2, 4, 7, | IP |
| | | 10 | |
| XMT-1519 | 4.0 mg/kg + | qwx2 + qd at days 0, | IV + IP |
| conjugate + | 10.0 mg/kg | 2, 4, 7, 10 | |
| PD-1 Concurrent | | | |
| XMT-1519 | 4.0 mg/kg + | qwx2 + qd at days 0, | IV + IP |
| conjugate + | 10.0 mg/kg | 2, 4, 7, 10 | |
| anti-PD-1 mAb | | | |
| 4 Days later | | | |
| Anti-PD-1 mAb + | 4.0 mg/kg + | qwx2 (starting at | IV + IP |
| XMT-1519 conjugate | 10.0 mg/kg | D4) + qd at days 0, | |
| 4 Days later | | 2, 4, 7, 10 | |
| Kadcyla + anti-PD-1 | 15.0 mg/kg + | qwx2 + qd at days 0, | IV + IP |
| mAb Concurrent | 10.0 mg/kg | 2, 4, 7, 10 | |

FIG. 6 shows the tumor response in mice after treatments with different regimens. As seen in Example 5, a combination of XMT-1519 conjugate and anti-PD1 mAb therapy when administered concurrently, lead to significant reduction in tumor growth in vivo along with complete response in one mouse; administration of the anti-PD1 mAb therapy followed by the administration of the XMT-1519 conjugate 4 days later lead to with complete responses in two mice; and administration of the XMT-1519 conjugate followed by the administration of the anti-PD1 mAb therapy 4 days later lead to with complete responses in three mice. Importantly, the frequency of complete responders was further increased when the two drugs were administered sequentially, rather than concurrently, such that XMT-1519 conjugate administration was followed by anti-PD1 mAb therapy 4 days later. These results could suggest an immunological mechanism involving induction of immunogenic cell death by XMT-1519 conjugate, which in turn may activate the adaptive immune system by releasing tumor specific antigens.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A combination comprising a HER2-targeted antibody-drug conjugate and an immune checkpoint inhibitor, wherein the conjugate comprises an antibody or antigen binding fragment thereof that specifically binds to an epitope of the human HER2 receptor and one or more therapeutic or diagnostic agents (D), wherein each D is independently connected directly or indirectly to the antibody or antigen binding fragment thereof, and wherein the antibody or antigen binding fragment thereof has the epitopic specificity of, or competes for binding HER2 with, an antibody comprising a CDRH1 comprising the amino acid sequence FTFSSYSMN (SEQ ID NO: 25); a CDRH2 comprising the amino acid sequence YISSSSSTIYYADSVKG (SEQ ID NO: 26); a CDRH3 comprising the amino acid sequence GGHGYFDL (SEQ ID NO: 27); a CDRL1 comprising the amino acid sequence RASQSVSSSYLA (SEQ ID NO: 28); a CDRL2 comprising the amino acid sequence GASSRAT (SEQ ID NO: 21); and a CDRL3 comprising the amino acid sequence QQYHHSPLT (SEQ ID NO: 29).

2. The combination of claim 1, wherein the antibody or antigen binding fragment thereof comprises a CDRH1 comprising the amino acid sequence FTFSSYSMN (SEQ ID NO: 25); a CDRH2 comprising the amino acid sequence YISSSSSTIYYADSVKG (SEQ ID NO: 26); a CDRH3 comprising the amino acid sequence GGHGYFDL (SEQ ID NO: 27); a CDRL1 comprising the amino acid sequence RASQSVSSSYLA (SEQ ID NO: 28); a CDRL2 comprising the amino acid sequence GASSRAT (SEQ ID NO: 21); and a CDRL3 comprising the amino acid sequence QQYHHSPLT (SEQ ID NO: 29).

3. The combination of claim 1, wherein the immune checkpoint inhibitor is a therapeutic biologic a small molecule, a monoclonal antibody, a humanized antibody, a fully human antibody, a fusion protein or a combination thereof.

4. (canceled)

5. The combination of claim 1, wherein the immune checkpoint inhibitor inhibits a checkpoint protein or interacts with a ligand of a checkpoint protein that comprises CTLA-4, PDL1, PDL2, PD1, BTLA, HVEM, TIM3, GAL9, LAG3, VISTA, KIR, 2B4, CD160, CGEN-15049, CHK1, CHK2, A2aR, a B-7 family ligand, CD2, CD27, CD28, CD30, CD40, CD70, CD80, CD86, CD137, CD226, CD276, DR3, GITR, HAVCR2, HVEM, IDO1, IDO2, ICOS (inducible T cell costimulator), LAIR1, LIGHT, MARCO (macrophage receptor with collagenous structure), OX-40, SLAM, TIGHT, VTCN1 or a combination thereof.

6-7. (canceled)

8. The combination of claim 5, wherein the immune checkpoint inhibitor inhibits a checkpoint protein that comprises CTLA-4, PDL1, PD1, or a combination thereof.

9. The combination claim 1, wherein the immune checkpoint inhibitor comprises pembrolizumab (MK-3475), nivolumab (BMS-936558), pidilizumab (CT-011), AMP-224, MDX-1 105, durvalumab (MEDI4736), MPDL3280A, BMS-936559, IPH2101, TSR-042, TSR-022, ipilimumab, lirilumab, atezolizumab, avelumab, tremelimumab, or a combination thereof.

10. The combination of claim 1, wherein the immune checkpoint inhibitor comprises nivolumab (BMS-936558), ipilimumab, pembrolizumab, atezolizumab, tremelimumab, durvalumab, avelumab, or a combination thereof.

11. The combination of claim 1, wherein the antibody or antigen binding fragment thereof of the conjugate comprises a variable heavy chain comprising the amino acid sequence of SEQ ID NO: 13 and a variable light chain comprising the amino acid sequence of SEQ ID NO: 14.

12. The combination of claim 1, wherein the antibody or antigen binding fragment thereof of the conjugate comprises a heavy chain comprising the amino acid sequence of SEQ ID NO: 5 and a light chain comprising the amino acid sequence of SEQ ID NO: 6.

13. The combination of claim 1, wherein the antibody or antigen binding fragment thereof of the conjugate is a monoclonal antibody, a domain antibody, a single chain antibody, a Fab fragment, a F(ab′)₂ fragment, a scFv, a scFv-Fc, a scAb, a dAb, a single domain heavy chain antibody, or a single domain light chain antibody.

14-16. (canceled)

17. The combination of claim 1, wherein the conjugate further comprises one or more polymeric scaffolds connected both to the antibody or antigen binding fragment thereof and to one or more D, wherein each of the one or more D is independently connected to the antibody or antigen binding fragment thereof via the one or more polymeric scaffolds.

18. The combination of claim 17, wherein each of the one or more polymeric scaffolds independently comprises poly(1-hydroxymethylethylene hydroxymethyl-formal) (PHF) having a molecular weight ranging from about 2 kDa to about 40 kDa.

19. The combination of claim 18, wherein each of the one or more polymeric scaffolds independently is of Formula (Ic): 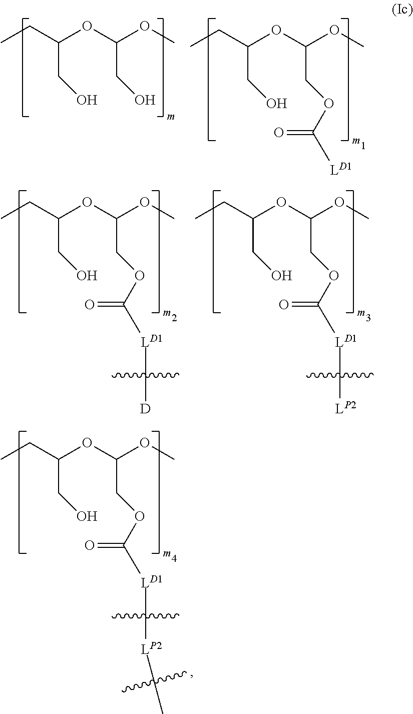
wherein:
L^{D1} is a carbonyl-containing moiety;
each occurrence of 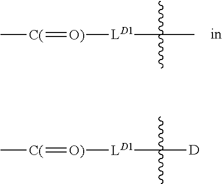
is independently a first linker that contains a biodegradable bond so that when the bond is broken, D is released in an active form for its intended therapeutic effect; and the 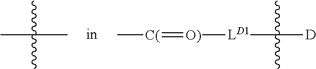
between L^{D1} and D denotes direct or indirect attachment of D to L^{D1};
each occurrence of 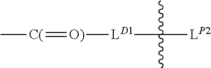
is independently a second linker not yet connected to the antibody or antigen binding fragment thereof, in which L^{P2} is a moiety containing a functional group that is yet to form a covalent bond with a functional group of the antibody or antigen binding fragment thereof, and the 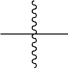
between L^{D1} and L^{P2} denotes direct or indirect attachment of L^{P2} to L^{D1}, and each occurrence of the second linker is distinct from each occurrence of the first linker;
each occurrence of 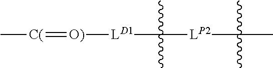
is independently a third linker that connects each D-carrying polymeric scaffold to the antibody or antigen binding fragment thereof, in which the terminal 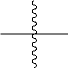
attached to L^{P2} denotes direct or indirect attachment of L^{P2} to the antibody or antigen binding fragment thereof upon formation of a covalent bond between a functional group of L^{P2} and a functional group of the antibody or antigen binding fragment thereof; and each occurrence of the third linker is distinct from each occurrence of the first linker;
m is an integer from 1 to about 300,
m₁ is an integer from 1 to about 140,
m₂ is an integer from 1 to about 40,
m₃ is an integer from 0 to about 18,
m₄ is an integer from 1 to about 10;
the sum of m, m₁, m₂, m₃, and m₄ ranges from 15 to 300; and
the total number of L^{P2} connected to the antibody or antigen binding fragment thereof is 10 or less.

20-21. (canceled)

22. The combination of claim 19, wherein the sum of m, m₁, m₂, m₃ and m₄ ranges from 40 to 75, m₁ is an integer from 2 to 35, m₂ is an integer from 2 to 10, m₃ is an integer from 0 to 5; and PHF has a molecular weight ranging from about 5 kDa to about 10 kDa.

23. The combination of claim 19, wherein the functional group of L^{P2} is selected from —SR^{p}, —S—S-LG, 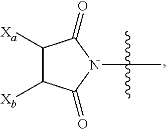
and halo, in which LG is a leaving group, R^{p} is H or a sulfur protecting group, and one of Xₐ and X_{b} is H and the other is a water-soluble maleimido blocking moiety, or Xₐ and X_{b}, together with the carbon atoms to which they are attached for a carbon-carbon double bond.

24. The combination of claim 19, wherein L^{D1} comprises —X—(CH₂)ᵥ—C(═O)— with X directly connected to the carbonyl group of 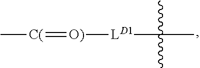
in which X is CH₂, O, or NH, and v is an integer from 1 to 6.

25. The combination of claim 19, wherein each occurrence of 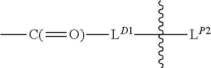
is independently —C(═O)—X—(CH₂)ᵥ—C(═O)—NH—(CH₂)ᵤ—NHC(═O)—(CH₂)_{w}—(OCH₂)ₓ—NHC(═O)—(CH₂)_{y}M, in which X is CH₂, O, or NH, each of v, u, w, x and y independently is an integer from 1 to 6, and M is 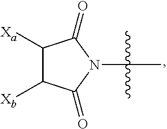
wherein one of Xₐ and X_{b} is H and the other is a water-soluble maleimido blocking moiety, or Xₐ and X_{b}, together with the carbon atoms to which they are attached for a carbon-carbon double bond.

26. The combination of claim 25, wherein each of v, u, w, x and y is 2.

27. The combination of claim 1, wherein in the conjugate each of the one or more D is a therapeutic agent having a molecular weight of ≤5 kDa, or at least one of the one or more D is a diagnostic agent.

28. The combination of claim 1, wherein at least one of the one or more D is an agent promoting immunogenic cell death comprises an anthracycline, an immunotoxin, doxorubicin, mitoxantrone, oxaliplatin, or bortezomib.

29-30. (canceled)

31. The combination of claim 17, wherein each of the one or more polymeric scaffolds independently is of Formula (If): 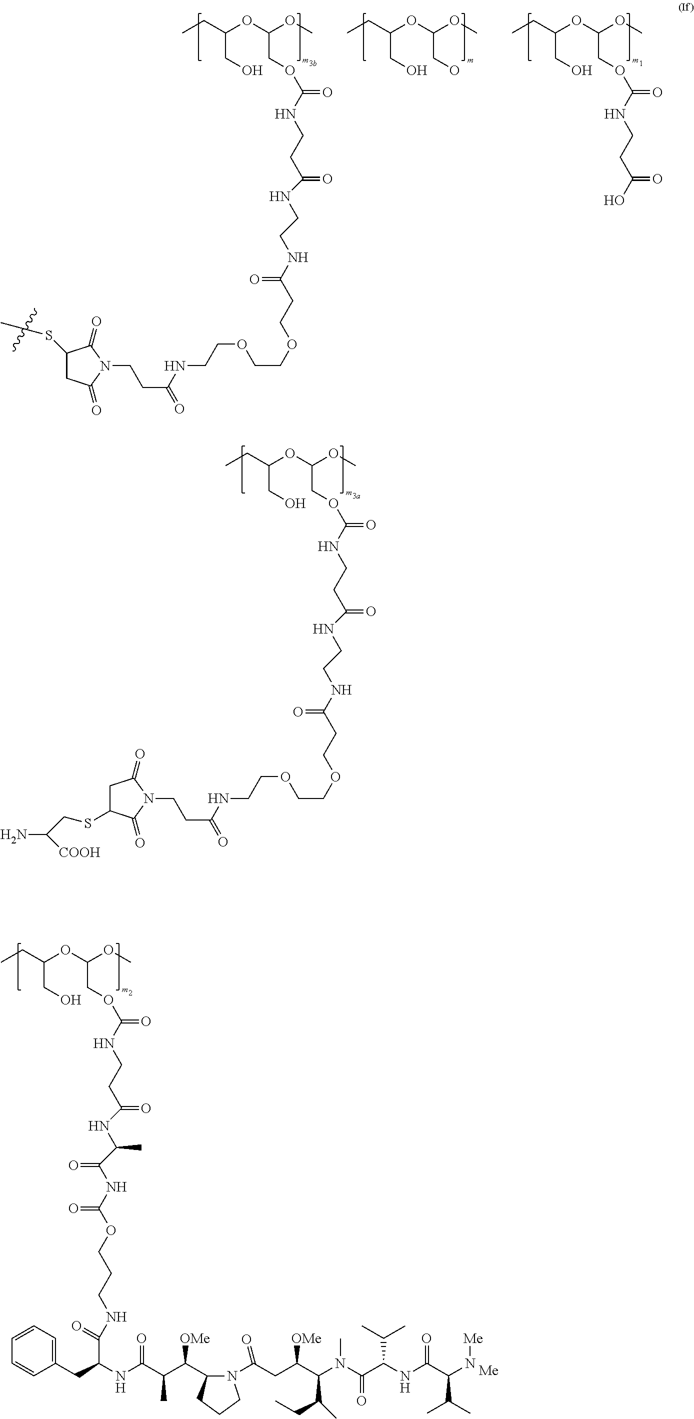
wherein:
m is an integer from 1 to about 300,
m₁ is an integer from 1 to about 140,
m₂ is an integer from 1 to about 40,
m₃ₐ is an integer from 0 to about 17,
m_{3b} is an integer from 1 to about 8;
the sum of m₃ₐ and m_{3b} ranges from 1 and about 18; and
the sum of m, m₁, m₂, m₃ₐ, and m_{3b} ranges from 15 to about 300;
the terminal 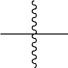
denotes the attachment of one or more polymeric scaffolds to the antibody or antigen binding fragment thereof that specifically binds to an epitope of the human HER2 receptor; and
the ratio between the PHF and the antibody is 10 or less.

32. The combination of claim 31, wherein the PHF in Formula (If) has a molecular weight ranging from about 2 kDa to about 20 kDa, the sum of m, m₁, m₂, m₃ₐ and m_{3b} ranges from about 15 to about 150, m₁ is an integer from 1 to about 70, m₂ is an integer from 1 to about 20, m₃ₐ is an integer from 0 to about 9, m_{3b} is an integer from 1 to about 8, the sum of m₃ₐ and m_{3b} ranges from 1 and about 10, and the ratio between the PHF and the anti-HER2 antibody is an integer from 2 to about 8.

33. The combination of claim 31, wherein the PHF in Formula (If) has a molecular weight ranging from about 3 kDa to about 15 kDa, the sum of m, m₁, m₂, m₃ₐ and m_{3b} ranges from about 20 to about 110, m₁ is an integer from 2 to about 50, m₂ is an integer from 2 to about 15, m₃ₐ is an integer from 0 to about 7, m_{3b} is an integer from 1 to about 8, the sum of m₃ₐ and m_{3b} ranges from 1 and about 8, and the ratio between the PHF and the anti-HER2 antibody or antigen-binding fragment thereof is an integer from 2 to about 8.

34. The combination of claim 1, wherein the conjugate and the immune checkpoint inhibitor are formulated in the same formulation.

35. The combination of claim 1, wherein the conjugate and the immune checkpoint inhibitor are formulated in separate formulations.

36. (canceled)

37. A method of preparing the combination of claim 1 for treating a HER2 expressing tumor in a subject in need thereof.

38. A kit comprising the combination of claim 1 and an instruction for administration.

39. A method of treating a HER2 expressing tumor in a subject in need thereof, the method comprising administering to the subject the combination of claim 1 in an amount sufficient to treat the HER2 expressing tumor.

40. The method of claim 39, wherein the subject is human.

41. The method of claim 39, wherein the tumor is selected from anal cancer, astrocytoma, leukemia, lymphoma, head and neck cancer, liver cancer, testicular cancer, cervical cancer, sarcoma, hemangioma, esophageal cancer, eye cancer, laryngeal cancer, mouth cancer, mesothelioma, skin cancer, myeloma, oral cancer, rectal cancer, throat cancer, bladder cancer, breast cancer, uterine cancer, ovarian cancer, prostate cancer, lung cancer, non-small cell lung cancer (NSCLC), colon cancer, pancreatic cancer, renal cancer, and gastric cancer.

42. The method of claim 41, wherein the tumor is selected from the group consisting of breast cancer, gastric cancer, non-small cell lung cancer (NSCLC), and ovarian cancer.

43. (canceled)

44. The method of claim 39, wherein the immune checkpoint inhibitor and the conjugate are administered simultaneously.

45. The method of claim 39, wherein the immune checkpoint inhibitor and the conjugate are administered sequentially in either order or in alternation.

46. The method of claim 45, wherein the conjugate is administered prior to the immune checkpoint inhibitor.

47. The method of claim 39, wherein the tumor is a HER2 positive cancer.

48. The method of claim 39, wherein the tumor is a HER2 negative cancer.

49. The method of claim 39, wherein the subject is identified as having low HER2 expression.

50. The method of claim 39, wherein the subject is identified as having a scoring of 1+ or 2+ for HER2 expression as detected by immunohistochemistry (IHC) analysis performed on a test cell population, and wherein the HER2 gene is not amplified in the test cell population.

51. The method of claim 39, wherein the subject is identified as having a scoring of 2+ or 3+ for HER2 expression as detected by immunohistochemistry (IHC) analysis performed on a test cell population, and wherein the HER2 gene is amplified or mutated in the test cell population.

52. The method of claim 39, wherein the immune checkpoint inhibitor and the conjugate show synergistic activity.

53-56. (canceled)

57. The combination of claim 31, wherein the PHF in Formula (If) has a molecular weight ranging from about 5 kDa to about 10 kDa, the sum of m, m₁, m₂, m₃ₐ and m_{3b} ranges from about 40 to about 75, m₁ is an integer from about 2 to about 35, m₂ is an integer from about 2 to about 10, m₃ₐ is an integer from 0 to about 4, m_{3b} is an integer from 1 to about 5, the sum of m₃ₐ and m_{3b} ranges from 1 and about 5; and the ratio between the PHF and the antibody is an integer from 2 to about 8
